(19) United States
(12) Patent Application Publication
(10) Pub. No.: US 2024/0287033 A1
(43) Pub. Date: Aug. 29, 2024

(54) DEGRADERS OF TISSUE TRANSGLUTAMINASE 2 (TG2)

(71) Applicant: Northwestern University, Evanston, IL (US)

(72) Inventors: Gary E. Schiltz, Evanston, IL (US); Daniela Elena Matei, Evanston, IL (US)

(21) Appl. No.: 18/433,123

(22) Filed: Feb. 5, 2024

Related U.S. Application Data
(60) Provisional application No. 63/483,188, filed on Feb. 3, 2023.

(51) Int. Cl. C07D 401/14; A61K 31/506; A61P 35/00; C07D 411/14

(52) U.S. Cl. CPC C07D 401/14; A61K 31/506; A61P 35/00; C07D 411/14

(57) ABSTRACT

Disclosed herein is a compound, or a pharmaceutically acceptable salt thereof, that has a formula M_{TG2}-L-M_{E3}. M_{TG2} is a moiety that binds to tissue transglutaminase 2 (TG2), L is a bond or a linker covalently attaching M_{TG2} and M_{E3}, and M_{E3} is a moiety that binds to an E3 ubiquitin ligase. Disclosed herein are also the uses of the compound, or a pharmaceutically acceptable salt thereof, and a pharmaceutical composition comprising the same, in a method of treating a disease or disorder associated with TG2 activity or in a method of inhibiting cell proliferation.

### CROSS-REFERENCE TO RELATED APPLICATIONS

This application claims benefit of priority to U.S. Application Ser. No. 63/483,188, filed Feb. 3, 2023, the content of which is incorporated by reference in its entirety.

### STATEMENT REGARDING FEDERALLY FUNDED RESEARCH

This invention was made with government support under grant numbers W81XWH-22-1-0470 and W81XWH-22-1-0471 awarded by the Department of Defense. The government has certain rights in the invention.

### BACKGROUND

Tissue transglutaminase (TG2) is a multi-functional enzyme involved in the crosslinking of extracellular matrix proteins, formation of a complex with fibronectin (FN) and integrins, and GTP hydrolysis. TG2 is activated in several pathological conditions including cancer. Over the past decade, TG2's role in cancer has been firmly established. The enzyme was found to be upregulated in several cancer types including ovarian, pancreatic, lung, breast cancer, and glioblastoma. TG2 expression was also correlated with poor clinical outcome in several cancer types, suggesting that it functions as a tumor promoter. Accordingly, TG2 degradation could profoundly affect cancer growth or survival in different cancer types.

Proteolysis-targeting chimeric molecules (PROTACs) are an emerging technology that may be utilized to target previously “undruggable” targets, transcription factors and non-enzymatic proteins. (See, e.g., An et al., “Small-molecule PROTACs: An emerging and promising approach for the development of targeted therapy drugs,” EBioMedicine. 2018 October; 36: 553-562; and Gu et al., “PROTACs: An Emerging Targeting Technique for Protein Degradation in Drug Discovery,” Bioessays. 2018 April; 40(4):el 700247, the contents of which are incorporated herein by reference in their entireties). PROTACs are chimeric molecules that may be characterized as “hetero-bifunctional” in that PROTACs include a ligand for recruiting an E3 ubiquitin ligase, a linker, and another ligand to bind with the protein targeted for degradation. Designed as such, PROTACs “hijack” the E3 ubiquitin ligase to the protein which is targeted for protein degradation via ubiquitination, even if the targeted protein is not a physiological substrate for degradation via the ubiquitin-proteasome system.

As such, novel PROTACs that induce degradation of tissue transglutaminase 2 (TG2) protein, as well as their use for treating diseases or disorders associated with TG2 activity (e.g. cancer), need to be developed.

### SUMMARY OF THE INVENTION

Disclosed herein is a compound having a formula M_{TG2}-L-M_{E3} and methods of using the same.

One aspect of the invention provides for a compound that has a formula M_{TG2}-L-M_{E3}, or a pharmaceutically acceptable salt thereof. M_{TG2} is a moiety that binds to tissue transglutaminase 2 (TG2), L is a bond or a linker covalently attaching M_{TG2} and M_{E3}, and M_{E3} is a moiety that binds to an E3 ubiquitin ligase.

Another aspect of the invention provides for a pharmaceutical composition comprising a therapeutically effective amount of the compound as disclosed herein, or a pharmaceutically acceptable salt thereof, and a pharmaceutically acceptable carrier, excipient, or diluent.

Another aspect of the invention provides for a method of treating a disease or disorder associated with TG2 activity in a subject in need thereof. The method comprises administering to the subject the compound as disclosed herein, or a pharmaceutically acceptable salt thereof, or the pharmaceutical composition as disclosed herein.

Another aspect of the invention provides for a method of inhibiting cell proliferation. The method comprises contacting cells with the compound as disclosed herein, or a pharmaceutically acceptable salt thereof, or the pharmaceutical composition as disclosed herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows structures of known TG2 inhibitors.

FIG. 2 shows structures of TG2 PROTACs.

FIGS. 3A-3N show characterization of TG2 degradation in OVCAR 5 and SKOV3 cells. FIG. 3A shows western blot analysis of TG2 in OVCAR-5 cells treated with Compounds 7 and 11 at 0.1, 1 and 10 μM at 6 hours. DMSO was used as a control. FIG. 3B demonstrates western blot analysis of TG2 in SKOV3 cells treated with Compounds 7 and 11 for 6 hours at 1 and 10 μM. FIG. 3C shows western blot analysis of TG2 in OVCAR-5 cells treated with Compounds 7 and 11 at 0.1, 1 and 10 μM at 24 hours. DMSO was used as a control. FIG. 3D shows western blot analysis of TG2 in SKOV3 cells treated with Compounds 7 and 11 for 24 hours at 1 and 10 μM. FIG. 3E demonstrates kinetics of TG2 degradation with Compound 7 based on western blot analysis in SKOV3 cells. Western blot against TG2 were performed at 0, 2, 6, 12 and 24 hrs. using 0.1, 0.3, 1, 3, 10 and 30 μM of Compound 7. Maximum inhibition was observed at 6 hrs using the highest concentrations. FIG. 3F demonstrates kinetics of TG2 degradation with Compound 11 based on western blot analysis in SKOV3 cells. Western blot against TG2 were performed at 0, 2, 6, 12 and 24 hrs. using 0.1, 0.3, 1, 3, 10 and 30 μM of Compound 11. Maximum inhibition was observed at 6 and 12 hrs using the highest concentrations. FIG. 3G shows western blot analysis of Compounds 7 dosed every 6-8 hours over 24 hrs. in OVCAR5 cells at doses of either 1 μM or 10 μM. FIG. 3H shows western blot analysis of Compounds 7 dosed every 6-8 hours over 24 hrs. in SKOV3 cells at doses of either 1 μM or 10 μM. FIG. 3I shows cell viability assay of OVCAR-5 and SKOV3 cells treated with Compound 7; no cell death was observed in either cell line using 0.1, 0.3, 1, 3, 10 and 30 μM over the course of 5 days. FIG. 3J demonstrates cell viability assay of OVCAR-5 and SKOV3 cells treated with Compound 11; no cell death was observed in either cell line using 0.1, 0.3, 1, 3, 10 and 30 μM over the course of 5 days. FIG. 3K demonstrates quantification of western blots shown in FIG. 3A (n=3 experimental replicates; *p<0.05; ***p<0.001; ****p<0.0001). FIG. 3L shows quantification of western blots shown in Figure B (n=3 experimental replicates; **p<0.01). FIG. 3M illustrates quantification of western blots shown in Figure C (n=3 experimental replicates; n.s.). FIG. 3N illustrates quantification of western blot shown in Figure D (n=3 experimental replicates; n.s.).

FIGS. 4A-4L show TG2 degradation is proteosome-dependent. Both Compounds 7 and 11 were tested on OVCAR 5 and SKOV3 cells. FIG. 4A shows western blot analysis against TG2 in OVCAR-5 cells treated with 10 μM of Compounds 7 and 11 for 6 hrs. and the proteosome inhibitor MG132 (20 μM) for 8 hrs. Degradation of TG2 was observed with Compounds 7 and 11 but not co-treated with MG132. FIG. 4B demonstrates quantification of the western blot in FIG. 4A. **** denotes p<0.0001. FIG. 4C shows western blot analysis against TG2 in SKOV3 using the same conditions as in FIG. 4A. Degradation of TG2 was observed with Compounds 7 and 11 but not when combined with MG132. FIG. 4D demonstrates quantification of the western blot in FIG. 4B. **** denotes p<0.0001. FIG. 4E shows western blot analysis against TG2 in OVCAR-5 cells treated with 10 μM of Compounds 7 and 11 for 6 hrs. along with 10 μM of VHL ligand as a competitive inhibitor. Degradation of TG2 was observed with Compounds 7 and 11 but not when combined with the VHL ligand. FIG. 4F demonstrates quantification of the western blot in FIG. 4E. ** denotes p<0.01. FIG. 4G shows western blot analysis against TG2 in SKOV3 cells treated using the same conditions as in FIG. 4E. Degradation of TG2 was observed with Compounds 7 and 11 but not when combined with VHL ligand. FIG. 4H shows quantification of the western blot in FIG. 4G. ** denotes p<0.01; *** p<0.001. FIG. 4I shows western blot analysis using negative control Compounds 12 and 13 on OVCAR-5 cells. No degradation of TG2 was observed at 6 hrs. with either 1 or 10 PM. FIG. 4J demonstrates western blot analysis using negative control compounds 12 and 13 on SKOV3 cells. No degradation of TG2 was observed at 6 hrs. with either 1 or 10 μM. FIGS. 4K and 4L illustrate quantification of the western blot is shown in FIGS. 41 and 4J (n=3 replicates; n.s.).

FIGS. 5A-5E show that compounds 7 and 11 reduce the migration and adhesion capability of cancer cells. Cell migration was analyzed on both OVCAR-5 and SKOV3 cells. FIG. 5A shows wound healing assay of the OVCAR-5 cells treated with Compounds 7 and 11 and its quantification. Note the difference between the grey bars. * denotes p<0.05. FIG. 5B shows transwell migration assay of the OVCAR-5 cells treated with Compounds 7 and 11 and its quantification, * p<0.05. FIG. 5C shows wound healing assay of the SKOV3 cells treated with Compounds 7 and 11 and its quantification, * p<0.05. FIG. 5D demonstrates transwell migration assay of the SKOV3 cells, * p<0.05. FIG. 5E shows Adhesion assay of the OVCAR-5 and SKOV3 cells on fibronectin and in quantification. *** p<0.001; **** p<0.0001.

FIGS. 6A-6B illustrate isothermal calorimetry (ITC) binding studies of compounds to TG2. FIG. 6A shows calorimetric titrations of 100 μM MT4 into 2 μM TG2 at 30° C.; each peak corresponds to a single injection of 2 μL. Binding of MT4 to TG2 shows a KD=7.8 μM. The c value for this experiment is 0.26. FIG. 6B shows calorimetric titrations of 200 μM 11 into 20 μM TG2 at 30° C.; each peak corresponds to a single injection of 2 μL. The binding of 11 to TG2 shows a KD=68.9 μM. The c value for this experiment is 0.29.

FIGS. 7A-7B illustrate docking of inhibitor MT4 into TG2. FIG. 7A shows the structure of TG2 from PDB 4PYG was used to docked MT4 into the fibronectin-binding site. Residues within 8 Å of MT4 are shown and interacting amino acids are labelled. Key potential interactions are highlighted with dashed lines, including hydrogen-bonds with Asp94 and Ser101. FIG. 7B shows the docked pose from A shown in surface representation of TG2. The benzyl group is directed in towards the protein and sits in a hydrophobic pocket formed by Leu12, Leu14, and Trp40. The dimethylamino group appears positioned in a solvent-accessible position. Representations of the surface denote electronegative areas (e.g., aspartate), electropositive areas (e.g., lysine), and hydrophobic.

FIGS. 8A-8B show shRNA and CRISPR knockdown of TG2 reduce migration of OC cells. FIG. 8A shows western blot analysis of the expression of TG2 on OVCAR-5 shTG2 cells (4B12) and OVCAR-5 CRISPR knockout cells (4C4) seeded on plastic (UN) and fibronectin (FN). FIG. 8B demonstrates wound healing assay of the OVCAR-5 cells parental, 4B12 and 4C4 together with its quantification.

FIGS. 9A-9C illustrate the phenotypical analysis of negative control compounds 12 and 13 on migration and adhesion capability of cancer cells. Cell migration was analyzed on both OVCAR-5 and SKOV3 cells. FIG. 9A demonstrates wound healing assay of the OVCAR-5 cells treated with Compounds 12 and 13 and its quantification. FIG. 9B shows wound healing assay of the SKOV3 cells treated with Compounds 12 and 13 and its quantification. FIG. 9C shows Adhesion assay of the OVCAR-5 and SKOV3 cells treated with Compounds 12 and 13 on fibronectin and quantification.

FIG. 10 illustrates calorimetric titrations of 200 μM PROTAC 7 into 20 μM TG2 at 30° C.; each peak corresponds to a single injection of 2 μL volume. No saturation was observed in this experiment—indicative of lower binding affinity (Kd>100 μM) for this compound.

FIGS. 11A-11B show degradation of TG2 in SKOV3 cells for compounds NUCC-0227323 (i.e., P23). Cells were treated with compound at the indicated concentrations for 24 hrs before western blot analysis to characterize the amount of TG2.

FIGS. 12A-12B show degradation of TG2 in SKOV3 cells for compounds NUCC-0227324 (i.e., P24) and NUCC-0227325 (i.e., P25). Cells were treated with compound for the indicated concentrations for 24 hrs before western blot analysis to characterize the amount of TG2.

FIG. 13 shows degradation of TG2 in SKOV3 cells treated for 6 hours with the indicated concentrations of compounds NUCC0227350 (i.e., P50) and NUCC0227351 (i.e., P51). Western blots were quantified by densitometry and graphed.

FIG. 14 shows degradation of TG2 in SKOV3 cells treated for 24 hours with the indicated concentrations of compounds NUCC0227350 (i.e., P50) and NUCC0227351 (i.e., P51). Western blots were quantified by densitometry and graphed.

FIG. 15 shows degradation of TG2 in SKOV3 cells treated for 24 hours with the indicated concentrations of compounds NUCC0227350 (i.e., P50) and NUCC0227351 (i.e., P51). Western blots were quantified by densitometry and graphed.

FIG. 16 shows degradation of TG2 in OVCAR5 cells treated for 6 hours with the indicated concentrations of compound NUCC-0227405 (i.e., P405). Western blots were quantified by densitometry and graphed.

FIG. 17 shows degradation of TG2 in OVCAR5 cells treated for 6 hours with the indicated concentrations of compound NUCC-0227405 (i.e., P405). Western blots were quantified by densitometry and graphed.

FIG. 18 shows degradation of TG2 in SKOV3 cells treated for 6 hours with the indicated concentrations of compounds NUCC-0227404 (i.e., P404) and NUCC0227405 (i.e., P405).

Western blots were quantified by densitometry and graphed.

FIG. 18 shows degradation of TG2 in SKOV3 cells treated for 24 hours with the indicated concentrations of compounds NUCC-0227404 (i.e., P404) and NUCC0227405 (i.e., P405). Western blots were quantified by densitometry and graphed.

FIG. 20 shows that compounds P404 and P405 reduce the migration and adhesion capability of cancer cells. Cell migration was analyzed in SKOV3 cells. FIG. 20 shows wound healing assay of the SKOV3 cells treated with Compounds P404 and P405 and its quantification. Note the difference between the grey bars. * denotes p<0.05. Shown is the wound healing assay of the SKOV3 cells treated with Compounds P404 and P405 and its quantification, * p<0.05. *** p<0.001; **** p<0.0001.

FIG. 21 shows degradation of TG2 in SKOV3 cells treated for either 6 hours (top) or 24 hrs (bottom) with the indicated concentrations of compound NUCC-0227374 (i.e., P74). Analysis was performed by western blot.

FIG. 22 shows degradation of TG2 in SKOV3 cells treated for either 6 hours (top) or 24 hrs (bottom) with the indicated concentrations of compound NUCC-0227374 (i.e., P74). Analysis was performed by western blot.

FIG. 23 shows that P404 and P405 degrade TG2 through the ubiquitin proteasome system because this degradation is blocked by the proteasome inhibitor MG132. SKOV3 cells were treated with 10 μM of compounds P404, P405, or MG132 for 6 hrs by themselves. SKOV3 cells were co-treated with P404 or P405 (10 μM) for 6 hrs along with MG132 (20 μM) for 8 additional hours. Cells were then analyzed by western blot.

FIG. 24 shows adhesion assay of SKOV3 cells treated with compounds 10 μM of compounds P404-P405 and quantified for the number of cells attached to a fibronectin surface.

### DETAILED DESCRIPTION OF THE INVENTION

The present invention is described herein using several definitions, as set forth below and throughout the application.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### Compounds

Disclosed herein are compounds, or pharmaceutically acceptable salts thereof, wherein the compounds have a formula M_{TG2}-L-M_{E3}. M_{TG2} is a moiety that binds to TG2. L is a bond or a linker covalently attaching M_{TG2} and M_{E3}. M_{E3} is a moiety that binds to an E3 ubiquitin ligase.

In some embodiments, M_{TG2} has a formula:

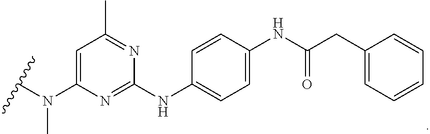 [SMILES: *N(C)c1cc(C)nc(Nc2ccc(NC(=O)Cc3ccccc3)cc2)n1]

In some embodiments, L comprises a polyethylene glycol moiety.

As used herein, the phrase “polyethylene glycol moiety” refers to a structural fragment of

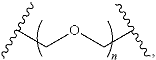

wherein n is an integer that is at least one. In some embodiments, n is an integer that is from 1 to 50. In some embodiments, n is an integer that is from 1 to 40, from 1 to 30, from 1 to 25, from 1 to 20, from 1 to 15, from 1 to 10, from 1 to 8, from 1 to 5, or from 1 to 3.

As used herein, a wavy line “ [symbol] ” may be used to designate the point of attachment for any radical group or substituent group.

In some embodiments, L is selected from the group consisting of

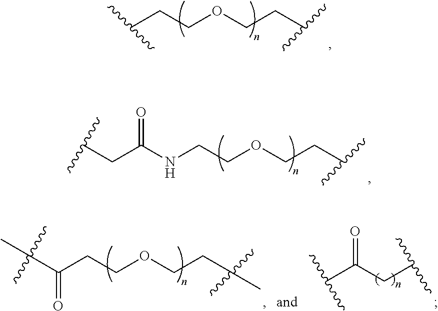

and wherein n is an integer from 1 to 20. In some embodiments, n is an integer from 1 to 15 or from 1 to 10. In some embodiments, n is an integer from 1 to 5.

In some embodiments, L is

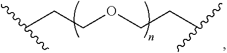

wherein n is an integer from 1 to 20, from 1 to 15, from 1 to 10, or from 1 to 5.

In some embodiments, L is

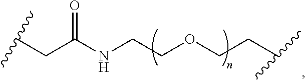

wherein n is an integer from 1 to 20, from 1 to 15, from 1 to 10, or from 1 to 5.

In some embodiments, L is

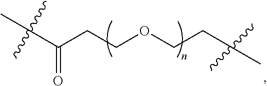

wherein n is an integer from 1 to 20, from 1 to 15, from 1 to 10, or from 1 to 5.

In some embodiments, L is

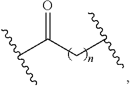

wherein n is an integer from 1 to 20, from 1 to 15, or from 1 to 10.

In some embodiments, L is selected from the group consisting of

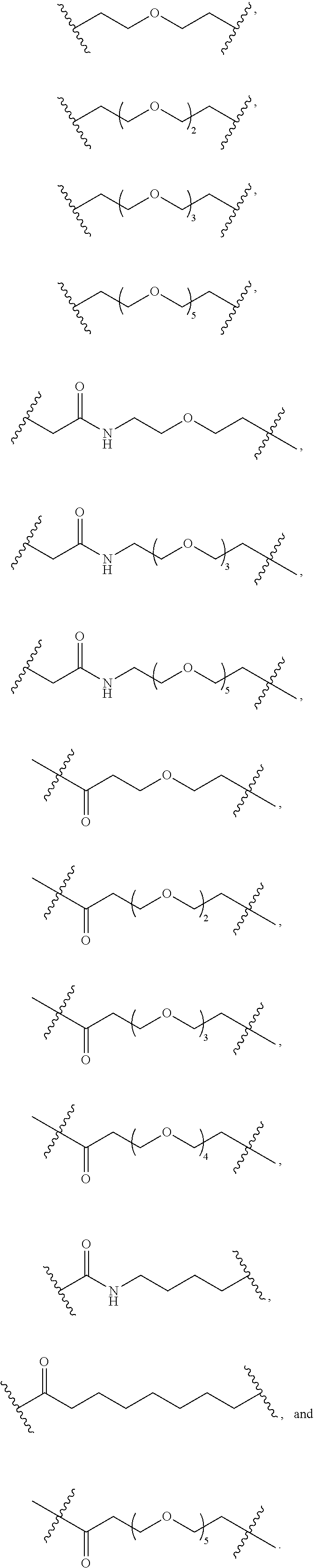

In some embodiments, M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase. In some embodiments, M_{E3} is a Von Hippel Lindau (VHL) E3 ubiquitin ligase. In some embodiments, M_{E3} is a cereblon (CRBN) E3 ubiquitin ligase.

In some embodiments, M_{E3} has a formula selected from the group consisting of:

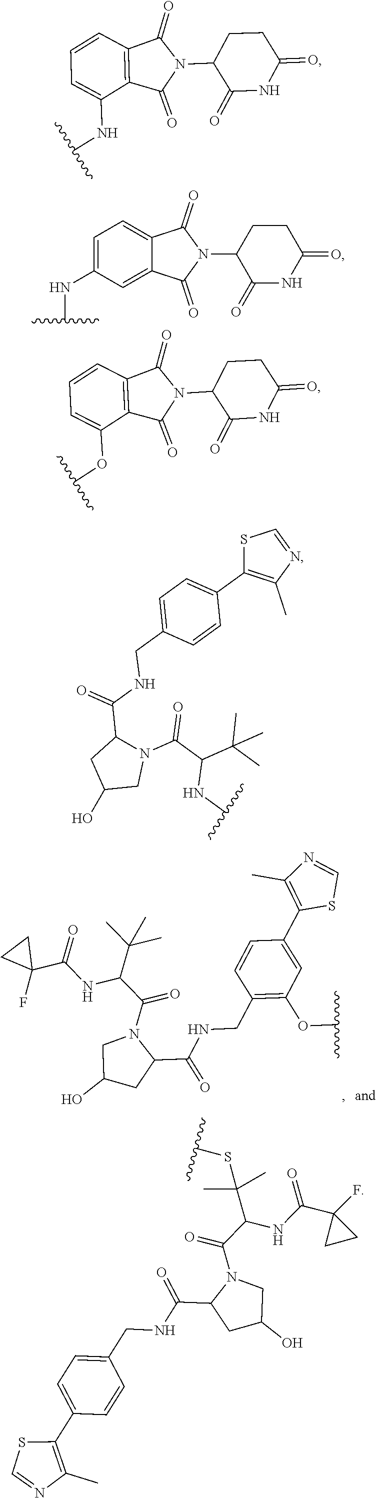

In some embodiments, the formula

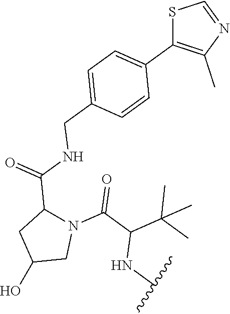 [SMILES: *NC(C(=O)N1CC(O)CC1C(=O)NCc1ccc(-c2scnc2C)cc1)C(C)(C)C]

comprises

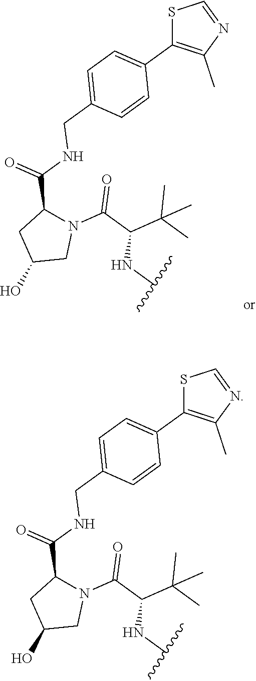 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@H]1C(=O)NCc1ccc(-c2scnc2C)cc1)C(C)(C)C]

In some embodiments, the formula

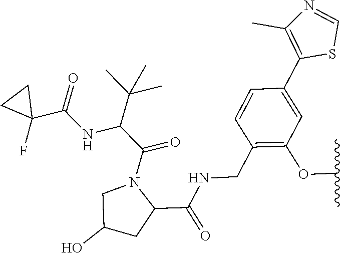 [SMILES: *Oc1cc(-c2scnc2C)ccc1CNC(=O)C1CC(O)CN1C(=O)C(NC(=O)C1(F)CC1)C(C)(C)C]

comprises

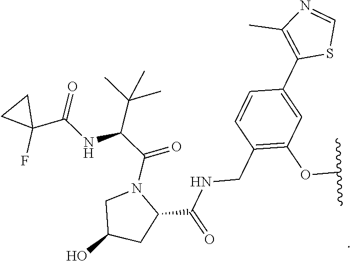 [SMILES: *Oc1cc(-c2scnc2C)ccc1CNC(=O)[C@@H]1C[C@@H](O)CN1C(=O)[C@@H](NC(=O)C1(F)CC1)C(C)(C)C]

In some embodiments, the formula

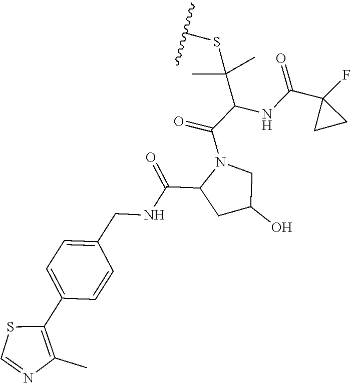 [SMILES: *SC(C)(C)C(NC(=O)C1(F)CC1)C(=O)N1CC(O)CC1C(=O)NCc1ccc(-c2scnc2C)cc1]

comprises

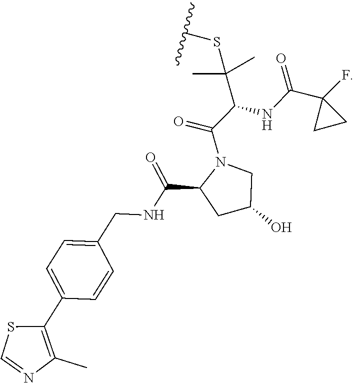 [SMILES: *SC(C)(C)[C@H](NC(=O)C1(F)CC1)C(=O)N1C[C@H](O)C[C@H]1C(=O)NCc1ccc(-c2scnc2C)cc1]

In some embodiments, the compound has a formula I

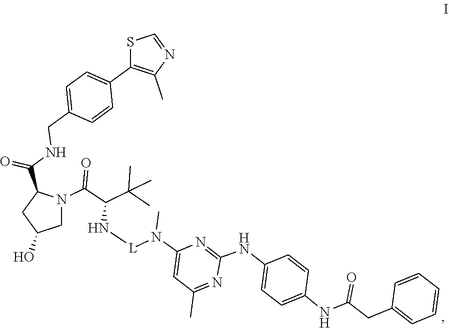

wherein L is as defined herein. In some embodiments, L is

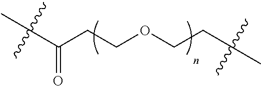

and n is an integer from 1 to 5.

In some embodiments, the compound has the formula M_{TG2}-L-M_{E3}, wherein: M_{TG2} has a formula

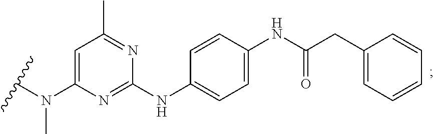 [SMILES: *N(C)c1cc(C)nc(Nc2ccc(NC(=O)Cc3ccccc3)cc2)n1]

L is selected from the group consisting of

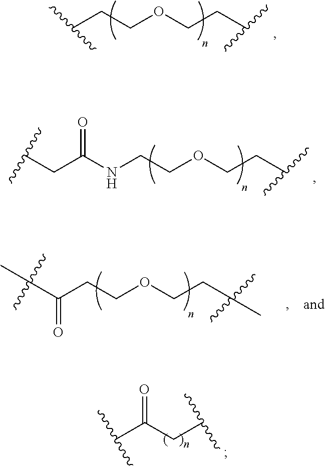

and n is an integer from 1 to 20; and
M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

In some embodiments, the compound has the formula M_{TG2}-L-M_{E3}, wherein:

M_{TG2} has a formula

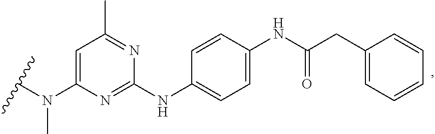 [SMILES: *N(C)c1cc(C)nc(Nc2ccc(NC(=O)Cc3ccccc3)cc2)n1]

L is selected from the group consisting of

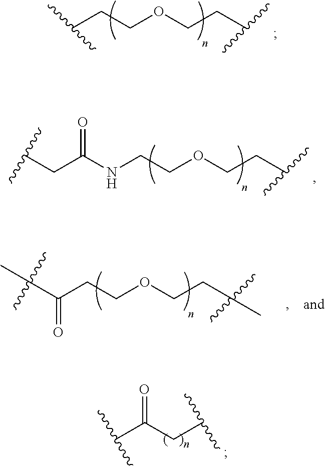

and n is an integer from 1 to 20; and
M_{E3} has a formula selected from the group consisting of

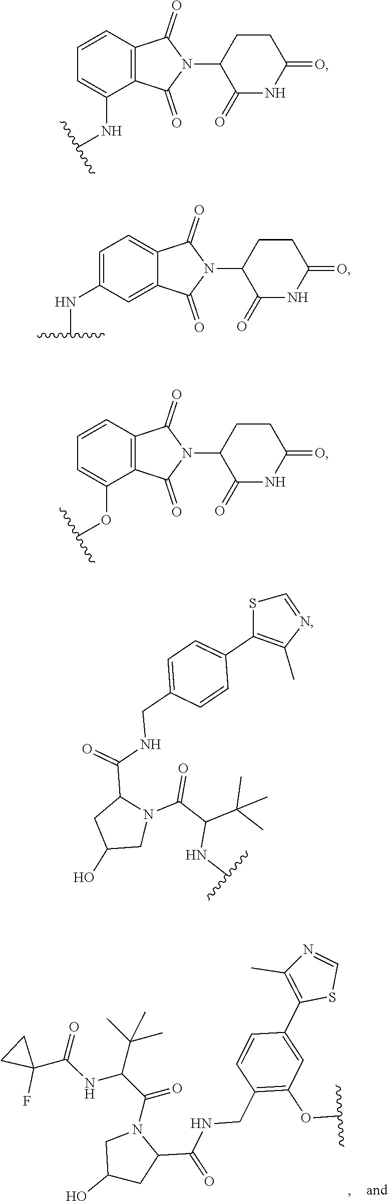 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *NC(C(=O)N1CC(O)CC1C(=O)NCc1ccc(-c2scnc2C)cc1)C(C)(C)C ; *Oc1cc(-c2scnc2C)ccc1CNC(=O)C1CC(O)CN1C(=O)C(NC(=O)C1(F)CC1)C(C)(C)C]

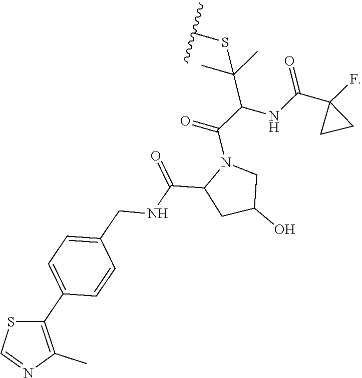 [SMILES: *SC(C)(C)C(NC(=O)C1(F)CC1)C(=O)N1CC(O)CC1C(=O)NCc1ccc(-c2scnc2C)cc1]

In some embodiments, the compound has the formula M_{TG2}-L-M_{E3}, wherein: M_{TG2} has a formula

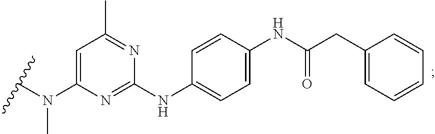 [SMILES: *N(C)c1cc(C)nc(Nc2ccc(NC(=O)Cc3ccccc3)cc2)n1]

L is selected from the group consisting of

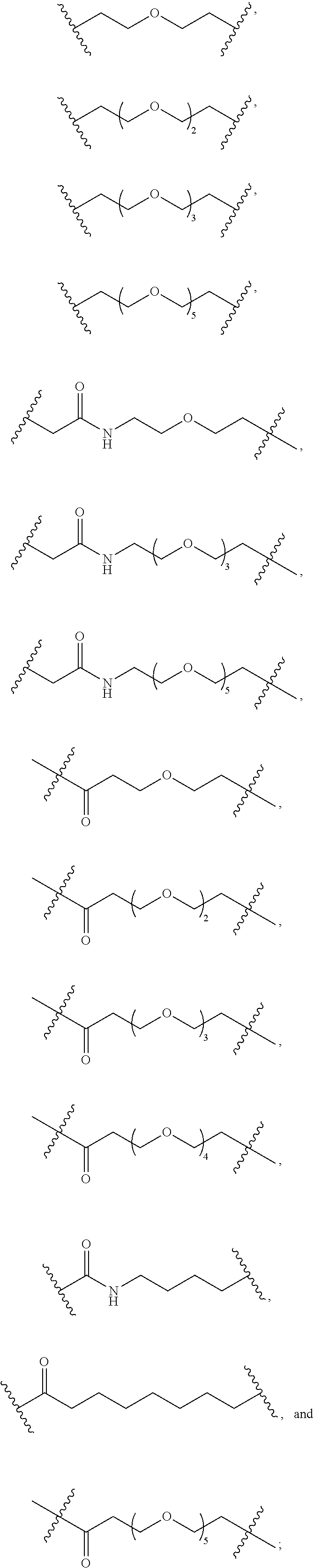

M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

In some embodiments, the compound has the formula M_{TG2}-L-M_{E3}, wherein:

M_{TG2} has a formula

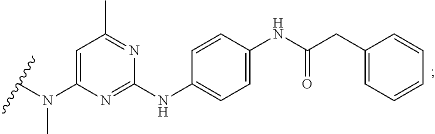 [SMILES: *N(C)c1cc(C)nc(Nc2ccc(NC(=O)Cc3ccccc3)cc2)n1]

L is selected from the group consisting of

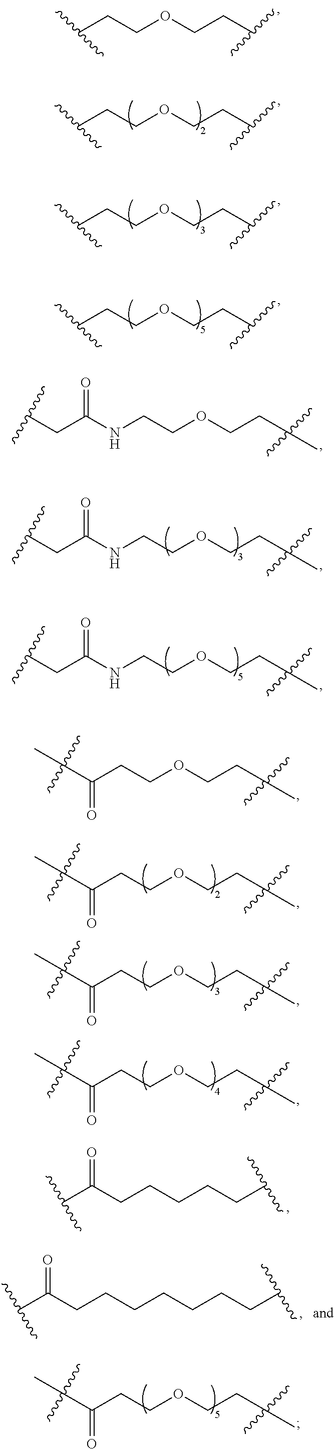

and M_{E3} has a formula selected from the group consisting of

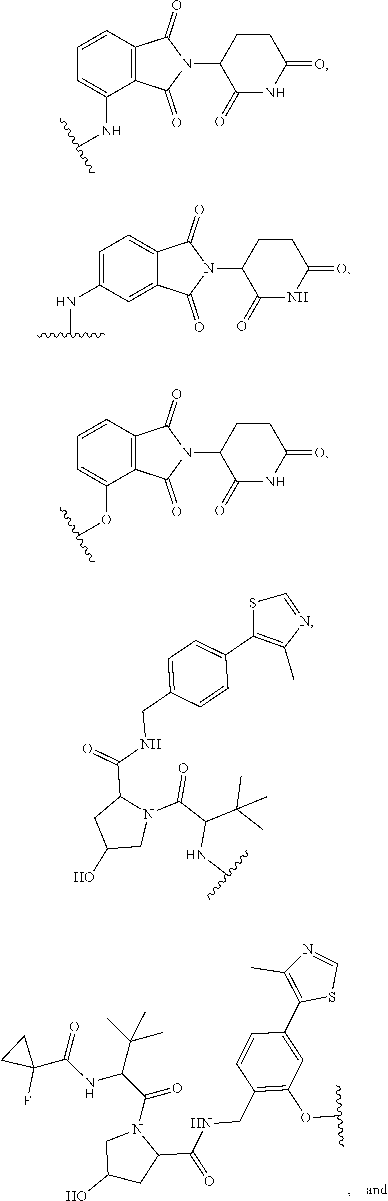 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *NC(C(=O)N1CC(O)CC1C(=O)NCc1ccc(-c2scnc2C)cc1)C(C)(C)C ; *Oc1cc(-c2scnc2C)ccc1CNC(=O)C1CC(O)CN1C(=O)C(NC(=O)C1(F)CC1)C(C)(C)C]

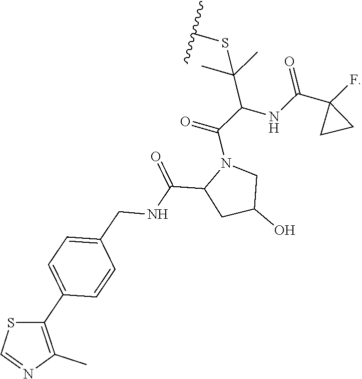 [SMILES: *SC(C)(C)C(NC(=O)C1(F)CC1)C(=O)N1CC(O)CC1C(=O)NCc1ccc(-c2scnc2C)cc1]

In some embodiments, the compound has a formula selected from the group consisting of

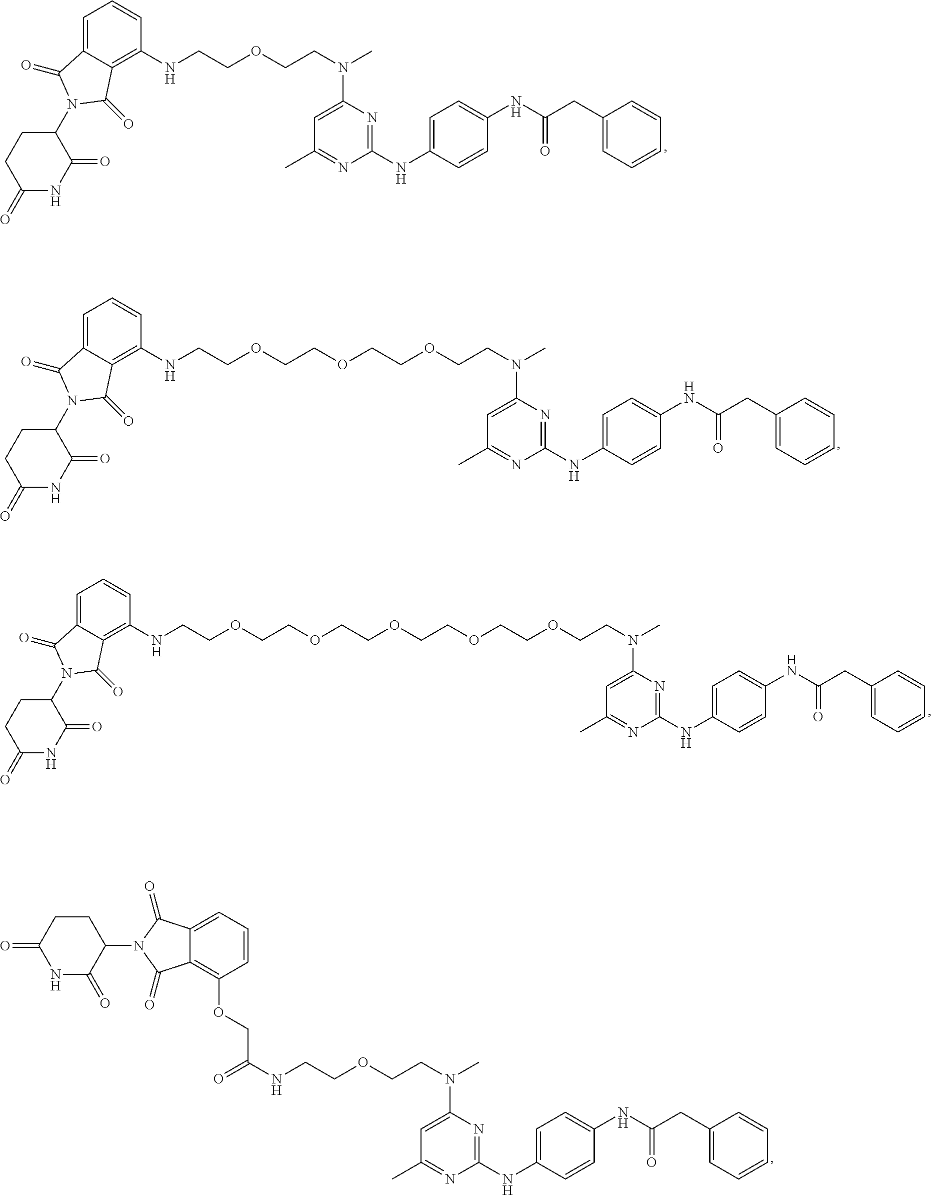 [SMILES: Cc1cc(N(C)CCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCNC(=O)COc2cccc3c2C(=O)N(C2CCC(=O)NC2=O)C3=O)nc(Nc2ccc(NC(=O)Cc3ccccc3)cc2)n1]

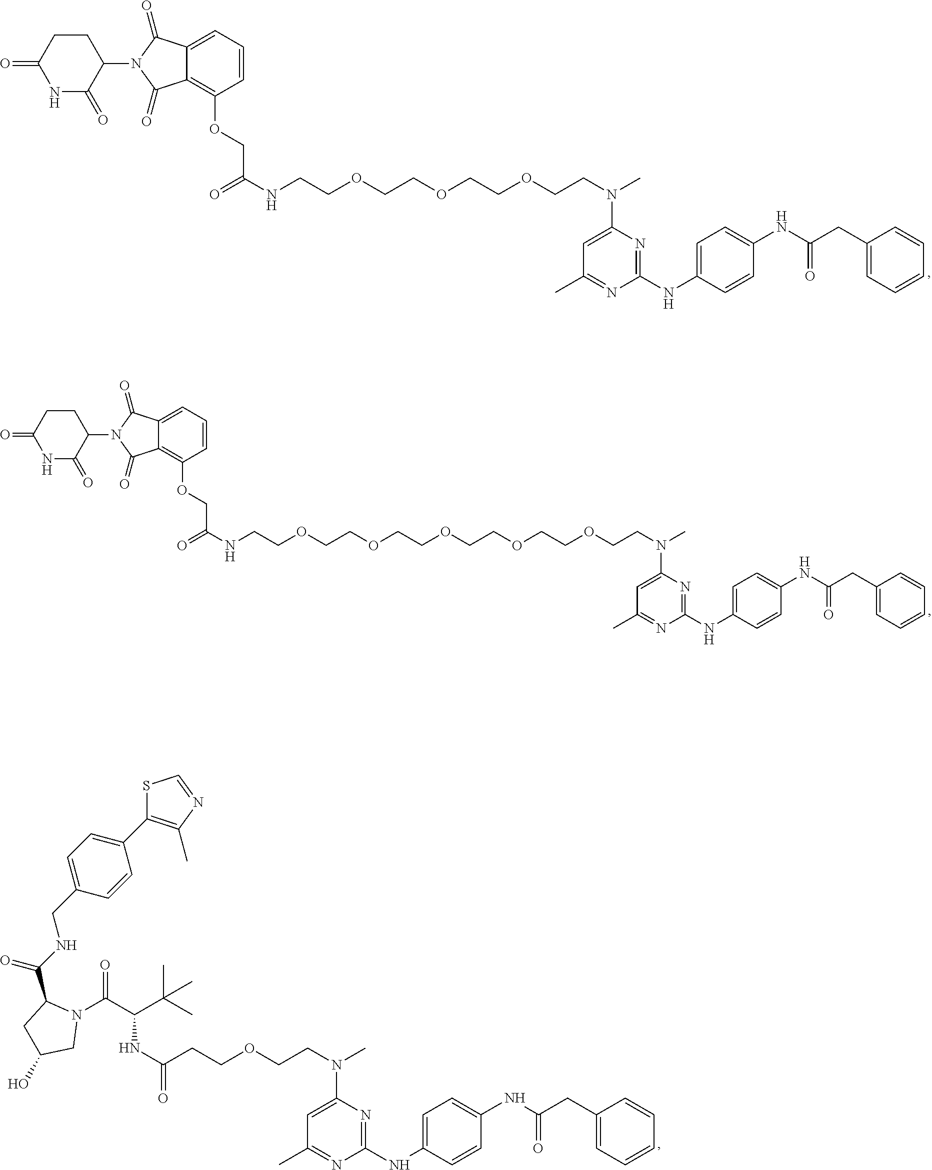 [SMILES: Cc1cc(N(C)CCOCCOCCOCCNC(=O)COc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNC(=O)COc2cccc3c2C(=O)N(C2CCC(=O)NC2=O)C3=O)nc(Nc2ccc(NC(=O)Cc3ccccc3)cc2)n1 ; Cc1cc(N(C)CCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

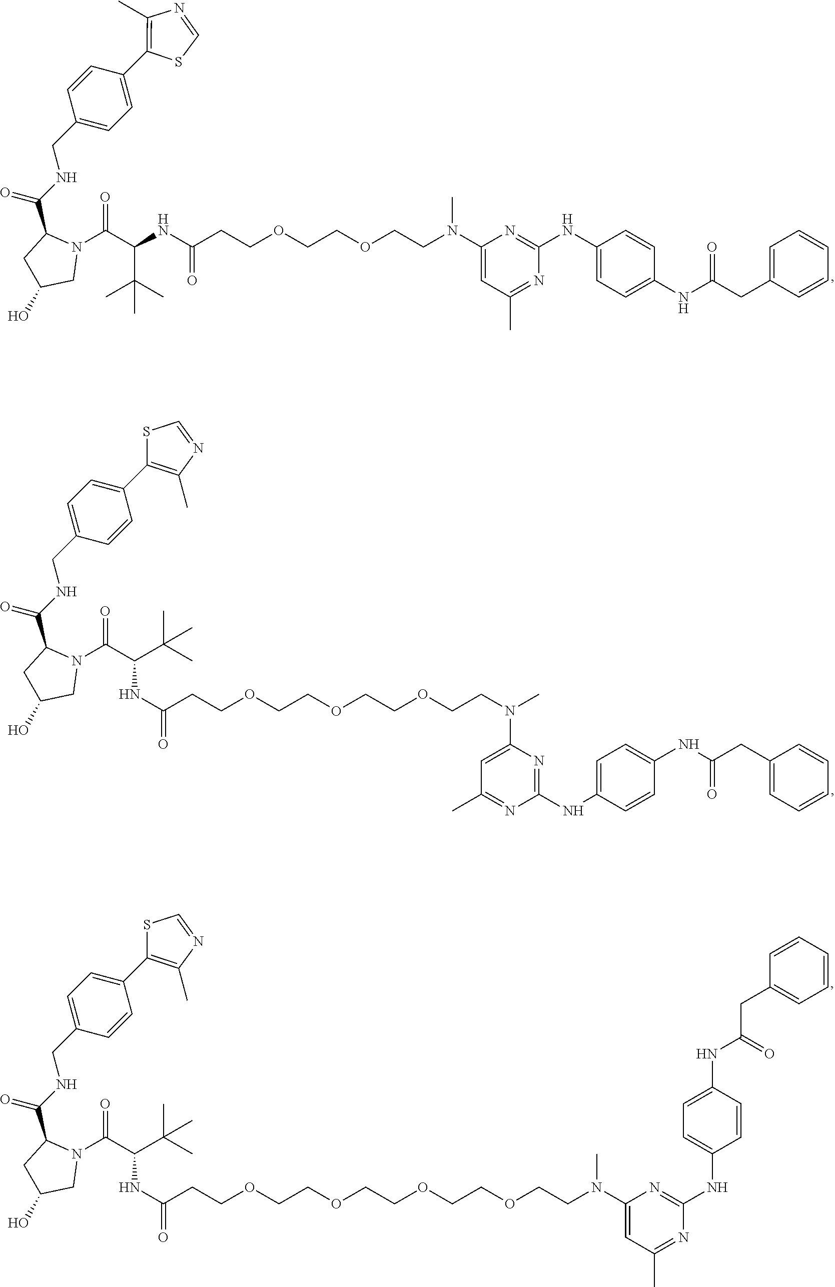 [SMILES: Cc1cc(N(C)CCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCOCCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

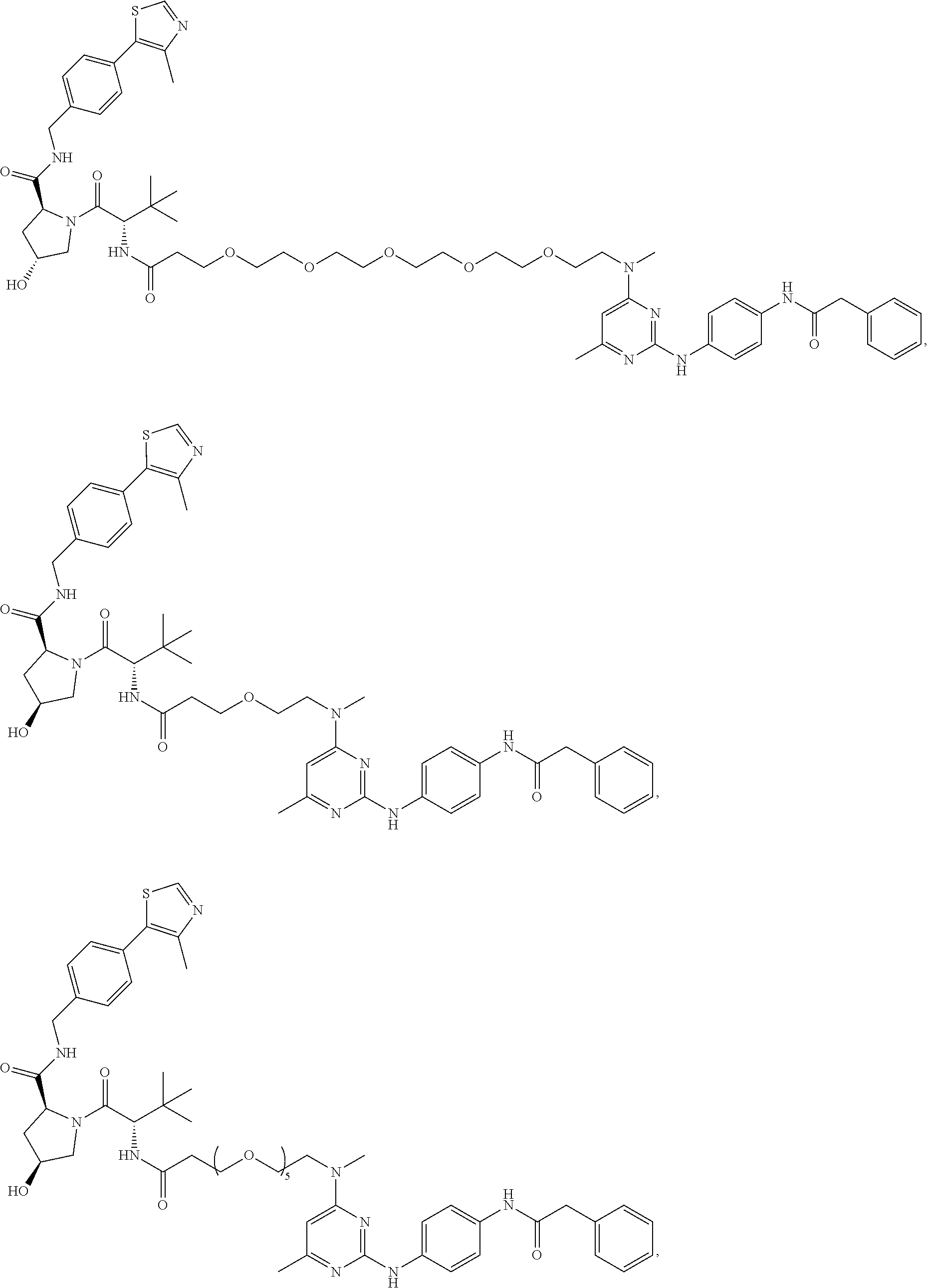

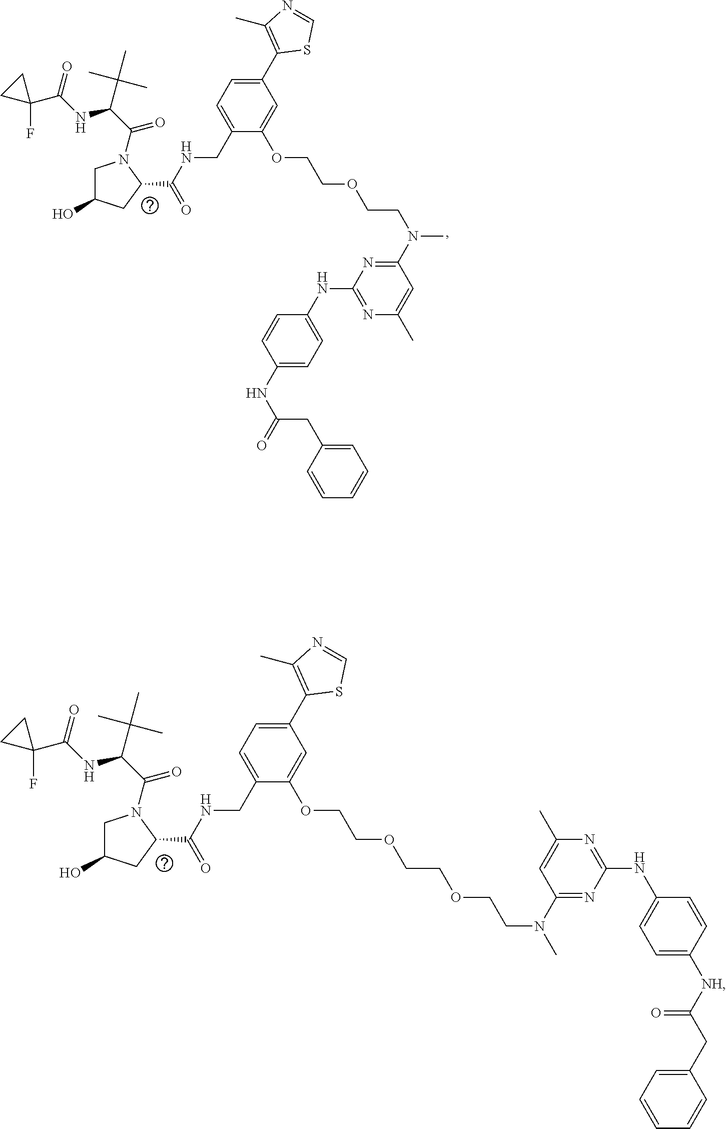 [SMILES: Cc1cc(N(C)CCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1 ; Cc1cc(N(C)CCOCCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1]

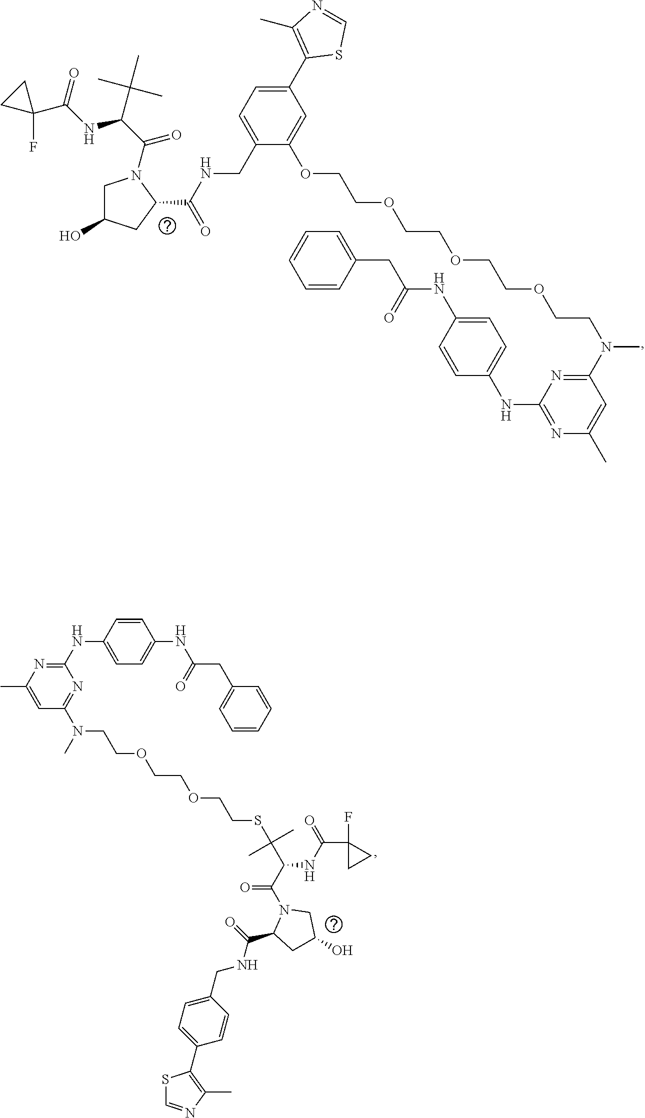

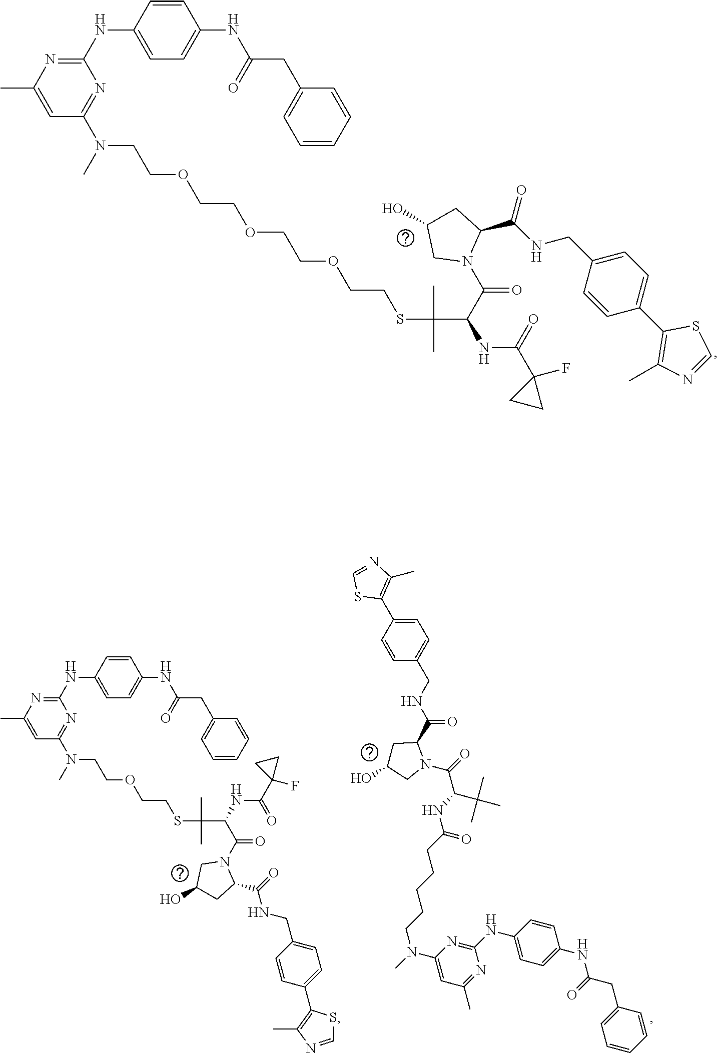 [SMILES: Cc1cc(N(C)CCOCCOCCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1 ; Cc1cc(N(C)CCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1 ; Cc1cc(N(C)CCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

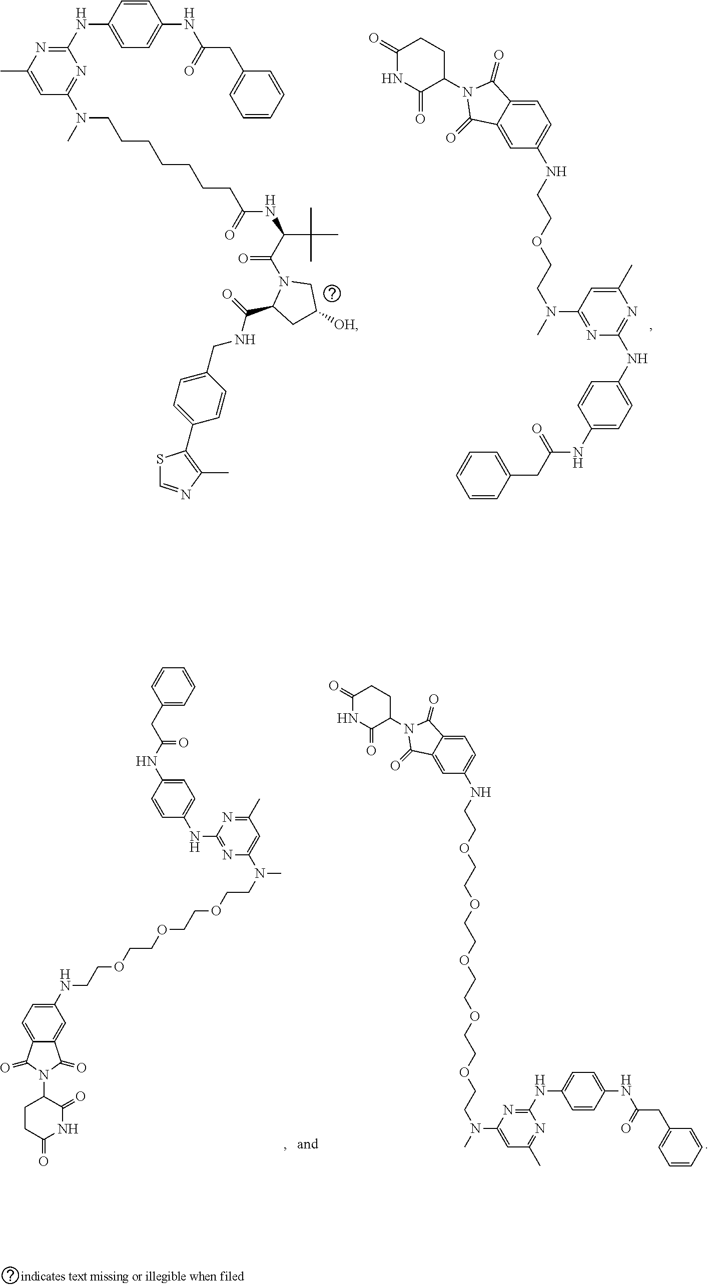 [SMILES: Cc1cc(N(C)CCCCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCNc2ccc3c(c2)C(=O)N(C2CCC(=O)NC2=O)C3=O)nc(Nc2ccc(NC(=O)Cc3ccccc3)cc2)n1 ; Cc1cc(N(C)CCOCCOCCOCCNc2ccc3c(c2)C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNc2ccc3c(c2)C(=O)N(C2CCC(=O)NC2=O)C3=O)nc(Nc2ccc(NC(=O)Cc3ccccc3)cc2)n1]

In some embodiments, the compound has a formula

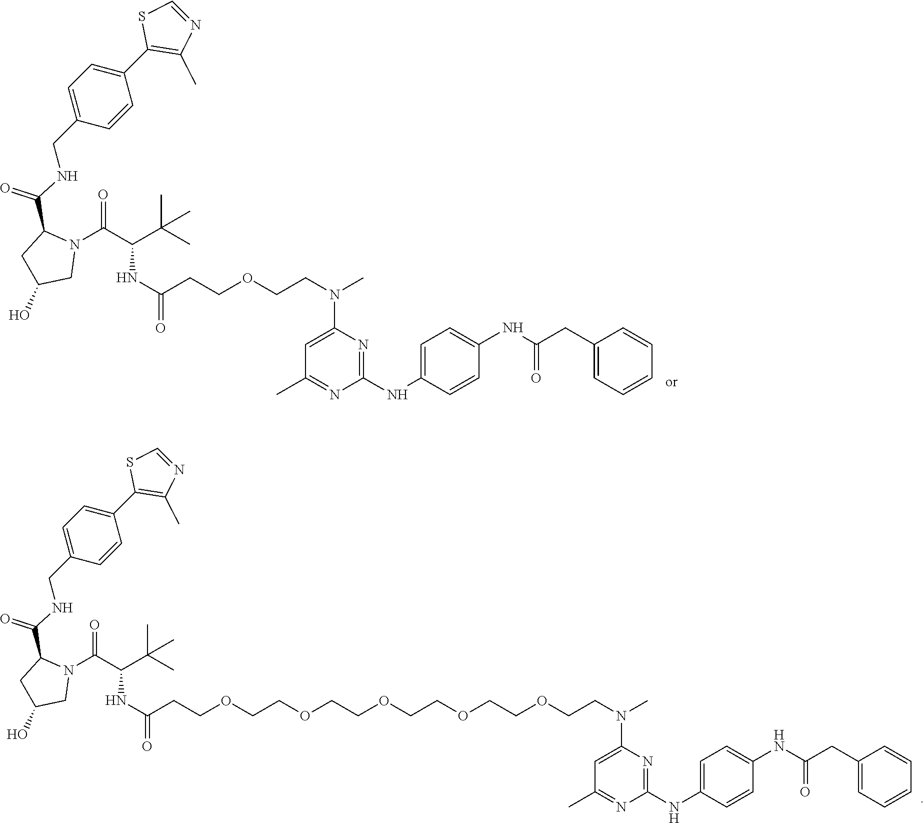 [SMILES: Cc1cc(N(C)CCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCOCCOCCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

### Pharmaceutical Compostions

Also disclosed herein are pharmaceutical composition comprising a therapeutically effective amount of the compounds as disclosed herein and a pharmaceutically acceptable carrier, excipient, or diluent.

As used herein, the phrase “effective amount” shall mean that drug dosage that provides the specific pharmacological response for which the drug is administered in a significant number of subject in need of such treatment. An effective amount of a drug that is administered to a particular subject in a particular instance will not always be effective in treating the conditions/diseases described herein, even though such dosage is deemed to be a therapeutically effective amount by those of skill in the art.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™) Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream.

When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cell proliferative diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition are formulated to comprise the disclosed compounds and further to comprise one or more additional therapeutic agents, for example, one or more additional therapeutic agents for treating cell proliferative diseases and disorders.

### Methods

Disclosed herein are also methods of treating a disease or disorder associated with TG2 activity in a subject in need thereof. The methods comprise administering to the subject the compound or the pharmaceutical composition as disclosed herein.

As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with the compounds as disclosed herein. For example, a “subject in need of treatment” may include a subject having a cell proliferative disease, disorder, or condition such as cancer. In some embodiments, the disease or disorder is a cell proliferative disease or disorder. In some such embodiments, the cell proliferative disease or disorder is ovarian cancer, melanoma, cervical cancer, colorectal cancer, liver cancer, meningioma, neuroblastomoa, prostate cancer, pancreatic cancer, lung cancer, breast cancer, or glioblastoma. In some embodiments, the cell proliferative disease or disorder is ovarian cancer.

A subject in need of treatment may also include a subject having a TG2 associated disease, disorder, or condition. Examples include chronic fibrosis or a neurodegenerative condition.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as endpoints any of these disclosed dose levels (e.g., 500-2000 ng/kg body weight of the subject)

Discloses herein are also methods of inhibiting cell proliferation. The methods comprise contacting cells with the compound as described herein, or a pharmaceutically acceptable salt thereof, or the pharmaceutical composition as described herein.

In some embodiments, the disclosed compound may be effective in inhibiting cell proliferation of one or more types of cancer cells including cells of ovarian cancer, melanoma, cervical cancer, colorectal cancer, liver cancer, meningioma, neuroblastomoa, prostate cancer, pancreatic cancer, lung cancer, breast cancer, or glioblastoma.

In some embodiments, the cells are ovarian cancer cells.

In some embodiments, the contact is in vitro.

Cell proliferation and inhibition thereof by the presently disclosed compounds may be assessed by cell viability methods disclosed in the art including colorimetric assays that utilize dyes such as MTT, XTT, and MTS to assess cell viability. In some embodiments, the disclosed compounds have an IC₅₀ of less than about 10 μM, 5 μM, 1 μM, or 0.5 μM in the selected assay.

### EXAMPLES

The following Examples are illustrative and are not intended to limit the scope of the claimed subject matter.

### Example 1

Because TG2 acts via multiple mechanisms, a targeted protein degradation strategy to abolish TG2's myriad functions was pursued. The synthesis and characterization of a series of VHL-based degraders that reduce TG2 expression in ovarian cancer cells in a proteasome-dependent manner were conducted. Degradation of TG2 results in significantly reduced cancer cell adhesion and migration in vitro in scratch-wound and migration assays. These results strongly indicate that further development of more potent and in vivo efficacious TG2 degraders may be a new strategy for reducing the dissemination of ovarian and other cancers.

Tissue transglutaminase (TG2) is a 77-kD protein belonging to the transglutaminase family, which includes transglutaminases 1-7, Factor XIII, and erythrocyte protein 4.2. These related proteins have similar catalytic activity; however, their substrate preferences and patterns of expression in tissues vary. TG2 has four domains: an N-terminus β-sandwich domain, which binds to fibronectin (FN), a catalytic domain that harbors the catalytic triad C²⁷⁷H³³⁵D³⁵⁸, which carries out the acyl-transfer function, and two β-barrel domains.^{1,2} A GTP/GDP-binding site is located between the catalytic and the first β-barrel domain. TG2 does not have a classical switch region, characteristic of G-proteins, and it remains unclear how its binding to GTP/GDP affects signaling. TG2 was shown to interact with PLCγ through a region located in its C-terminus and thus has been implicated in adrenergic signaling.³ The functions of the protein are modulated through allosteric changes, regulated by the concentrations of GTP and Ca²⁺. The crystal structure of GTP-bound TG2 (PDB 1KV3) has a compact conformation with the two β-barrel domains folding over the catalytic triad, obstructing the accessibility of Cys²⁷⁷ and blocking the enzymatic activity. The X-ray crystal structure of TG2 obtained in the presence of Ca²⁺ (PDB 2Q3Z) is open and exposes the catalytic core. In this “open” state, TG2 cannot bind GTP/GDP but interacts with substrates for transamidation.

Consequently, the physiological functions of TG2 are regulated by cellular context and localization. At the plasma membrane, TG2 binds to the 42 kDa gelatin-binding domain of FN (FN42) providing a binding site for β1 and β3 integrins⁴⁻⁶ and other membrane receptors such as PDGFR-β^{7, 8} and Frizzled-7.⁹ Mutations in the catalytic core have been shown to not affect formation of the FN/integrin/TG2 complex, proving that this function of the protein is independent of its transamidase role.⁵ Within the cytosol, where Ca²⁺ concentrations are low, the protein assumes a closed conformation, and binds to GTP and other partners, thereby altering cellular signaling.^{3, 10} In the extracellular matrix (ECM), where Ca²⁺ levels are high and nucleotide concentrations are low, TG2 functions as a transamidase, facilitating Ca²⁺-dependent incorporation of amines into proteins and acyl-transfer between glutamine and lysine residues, leading to protein cross-linking and facilitating matrix remodeling.¹¹ The catalytic activity of TG2 can also be inhibited by various oxidants through intramolecular disulfide bridge formation.^{12,13} Several matrix proteins have been shown to be TG2 substrates, including FN,¹⁴ fibrin,¹⁵ osteopontin,¹⁶ laminin,¹⁷ collagen,¹⁸ and others. These functions suggest that TG2 plays important roles in cell adhesion, migration, and stromal assembly, which are involved in fibrotic processes and cancer.

Over the past decade, TG2's role in cancer has been firmly established. The enzyme was found to be upregulated in ovarian,¹⁹²⁰ pancreatic,²¹ lung,²² breast cancer,²³ and glioblastoma.²⁴ TG2 expression was correlated with poor clinical outcomes in ovarian,²⁵ pancreatic,²⁶ and lung cancer,²⁷ suggesting that it functions as a tumor promoter. Functionally, TG2 was linked to chemotherapy resistance²⁸ through activation of the NF-xB survival pathway²⁹³⁰ and of “outside-in” signaling,²⁶ and with metastatic progression in ovarian cancer models¹⁹,2⁰ through induction of epithelial-to-mesen-chymal transition (EMT). More recently, TG2 was reported to be highly expressed in cancer stem cells (CSCs), facilitating their interaction with the metastatic niche and activating pro-survival pathways.³¹ While the pro-tumorigenic role of TG2 is clear, there is still a lack of consensus regarding which function and domain of the protein is responsible and should be targeted. We and others have shown that interactions with integrins and FN at the plasma membrane activate outside-in signaling (FAK, Akt, β-catenin, EGFR)^{32,33} and regulate cancer metastasis and stemness. However, in other contexts, the transamidase function was shown to have pro-tumorigenic functions: IκBα is cross-linked by TG2, leading to NF-xB activation;³⁴ RhoA is transamidated by TG2 and becomes constitutively active.³⁵ Other studies using TG2 mutants (R580A, which is unable to bind GTP; and C277, which is enzymatically inactive) suggested that the nucleotide-binding function of TG2 is required for maintaining cancer sternness.^{36,37} Thus, it remains unclear how to best target TG2, and the development of effective inhibitors has been challenging. Several strategies have been attempted, including small-molecule inhibitors for the TGase activity,^{38,39} for the TG2/FN complex,^{40,41} or for the GTPase function. While some anti-oncogenic activity was observed with each of the classes of proposed inhibitors, an active in vivo anti-TG2 strategy does not currently exist. This roadblock may be caused by the multifunctional properties of the protein and the need to block all its functions through a single modality.

A therapeutic modality that overcomes many of the challenges associated with difficult-to-target proteins is the PROteolysis-TArgeting Chimera (PROTAC) drugs, which act by inducing the degradation of a target protein.^{42,43} PROTACs are bifunctional molecules, with one end binding to a protein of interest and the other end binding to an E3 ubiquitin ligase. The PROTAC simultaneously binds to these two proteins to bring them into close proximity and form a ternary complex which facilitates the transfer of a ubiquitin molecule to the protein of interest and subsequent proteasomal degradation. The recent development of PROTACs as a new strategy to therapeutically target proteins has begun to significantly impact translational research.⁴⁴⁻⁴⁸ There are currently more than ten different PROTACs in clinical development.⁴⁴⁻⁵¹ Available human clinical data for these compounds have shown promising tolerability, pharmacokinetic (PK), and efficacy, further validating PROTACs as potential drugs.

There are several key advantages of PROTACs over traditional small-molecule inhibitors. First, it has been shown that degrading a target protein results in more intense and sustained inhibition of its functions.⁵² Second, because the compound is required to bind the target protein and the E3 ligase protein simultaneously and in the proper orientation, there is often greater selectivity regarding which proteins are degraded compared with which proteins are inhibited by the ligand. For instance, when promiscuous inhibitor warheads are used, typically, very few of the inhibited targets are degraded by the PROTACs because of the more stringent structural requirements of binding two proteins to form a ternary complex.⁴³ Third, many proteins, including TG2, have been shown to possess functions in addition to their enzymatic/signaling aspects, such as scaffolding partners. Existing inhibitors impact either TG2 enzymatic activity, or inhibit enzyme activity and GTP binding,⁵³ and others only suppress FN binding, while PROTAC-induced degradation can abolish all of a protein's functions and provide more functionally relevant effects.⁵⁴

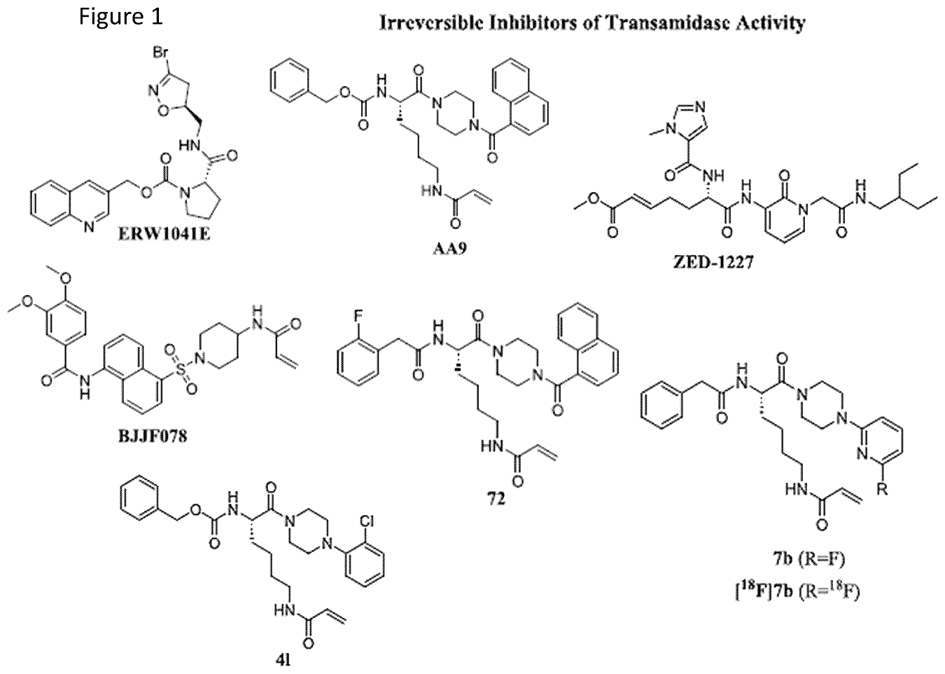

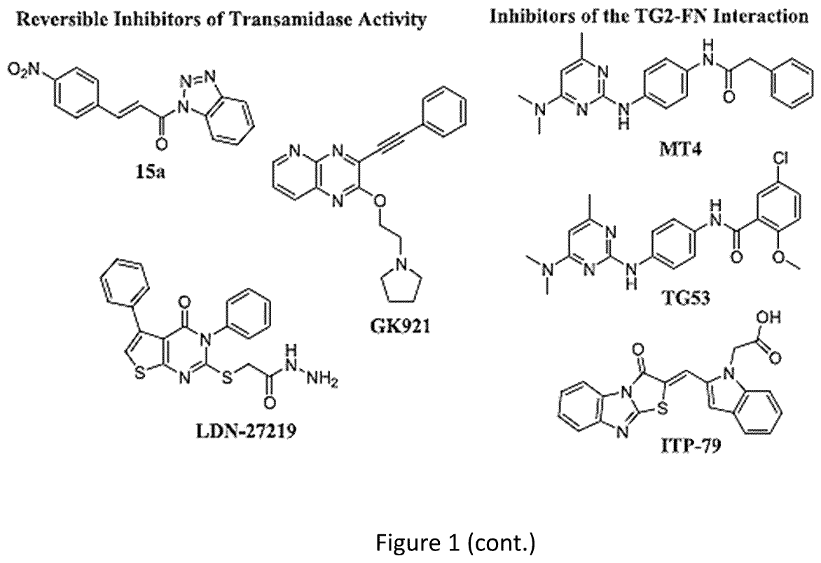

Several reports have studied covalent inhibitors, including bromodihydroisoxazole (DHI) derivatives and Michael acceptors.⁵⁵ In 2014, Khosla and co-workers reported structure-activity relationship (SAR) studies on various DHI analogs, in which the proline-containing inhibitor ERW1041E was used as a starting point for lead optimization to improve upon its potency, selectivity, and PK profile (FIG. 1).⁵⁶ More recently, van Dam and co-workers reported that Michael acceptor BJJF078, as well as ERW1041E, potently inhibited both mouse and human TG2 (and TG1) but did not inhibit the TG2-fibronectin interaction.⁵⁷ Several peptidomimetic inhibitors containing Michael acceptors have also been recently described, including ZED1227 from the biotech company Zedira, which is the first known TG2 inhibitor undergoing clinical trials.⁵⁸ Furthermore, there are several reports of Np-acryloyllysine irreversible inhibitors of TG2.

CHDI and Evotec reported irreversible TG2 inhibitors with an acrylamide moiety that were derived from a known lysine dipeptide.⁵⁹ Compounds in the series had several favorable properties, including improved polar surface area, excellent plasma stability, and improved potency relative to the benchmark dipeptide; however, they generally suffered from high P-gp efflux and a high rate of oxidative metabolism. The structures of one of these compounds, 4l, is shown above (FIG. 1). Akbar and co-workers also reported on NF-acryloyllysine irreversible inhibitors.⁵³ Novel inhibitors were developed that blocked both the transamidation function and GTP binding ability of hTG2. One of these compounds, AA9, was determined to have a good balance of affinity, efficiency, and physicochemical properties.

In later work, McNeil et al. reported compounds that retained the naphthoyl piperazine C-terminal functionality of AA9 but optimized the N-terminal functionality since substituents here were shown to contribute significantly to binding affinity.⁶⁰ Their efforts resulted in compounds like compound 72 with improved parameters over AA9, including fewer hydrogen-bond acceptors, lower polar surface area, and fewer rotatable bonds, all of which may help improve bioavailability. Wodtke et al. described N²⁴⁹-acryloyllysine piperazines in which substituents at the α-amino group and the piperazine ring were modified, resulting in compounds like 7b, which is a potent and selective TGase 2 inhibitor.⁶¹ In future work, this compound was modified to its 18F analogue to facilitate physiological studies of TGase 2 both in vitro and in vivo.⁶² Similarly, via a palladium-mediated [¹¹C]CO aminocarbonylation reaction, van der Wildt et al. described the synthesis of N²⁴⁹-acryloyllysine carbon-11-labeled PET tracers to further understand TG2 biology.⁶³

The first reported reversible inhibitors of TG2 contained a thieno[2,3-d]pyrimidin-4-one core.⁶⁴⁶⁵ Case and Stein later reported on compound LDN-27219, a reversible and slow-binding inhibitor that appeared to not bind to TG2's active site but at the GTP site.⁶⁶ Recent studies have also shown that competitive, reversible cinnamoyl-based inhibitors of TG2, like compound 15a, showed promising in vitro activity against TG2.⁶⁷ In 2014, Kim and co-workers demonstrated that reversible TG2 inhibitor GK921 showed promise against renal cell carcinoma in xenograft models.⁶⁸ There are several reports of compounds that disrupt the TG2/fibronectin interaction. Khanna et al. reported that compound ITP-79 was able to disrupt this interaction in ovarian cancer cells.⁶⁹ A series of TG2 inhibitors that are aminopyrimidine compounds, as exemplified by TG53 (FIG. 1), inhibited the TG2/fibronectin interaction and was initially identified using an AlphaLISA-based high-throughput screen. Subsequent work modified this initial hit to improve potency and solubility and resulted in inhibitor MT4.⁴¹

A protein degrader for TG2 could be developed which would abolish all of its pro-tumorigenic functions. This series of TG2-targeting protein degraders are described here. A small library of bifunctional molecules incorporating ligands for either cereblon (CRBN) or Von Hippel-Lindau (VHL) protein were prepared and they were connected via poly(ethylene glycol) (PEG) linkers to the TG2 ligand. Screening of these compounds for TG2 degradation in ovarian cancer cells identified two molecules that caused protein degradation. The proteasome-dependent mechanism of action of these compounds was confirmed using competition assays and negative control derivatives. Finally, as an in vitro proof of concept, these PROTACs were shown to inhibit TG2-mediated ovarian cancer migration. These results demonstrate the feasibility and utility of pursuing a targeted protein degradation approach for TG2 that has the potential to ameliorate ovarian cancer growth and metastasis.

### Results and Discussion

### Synthesis of new TG2 PROTACs

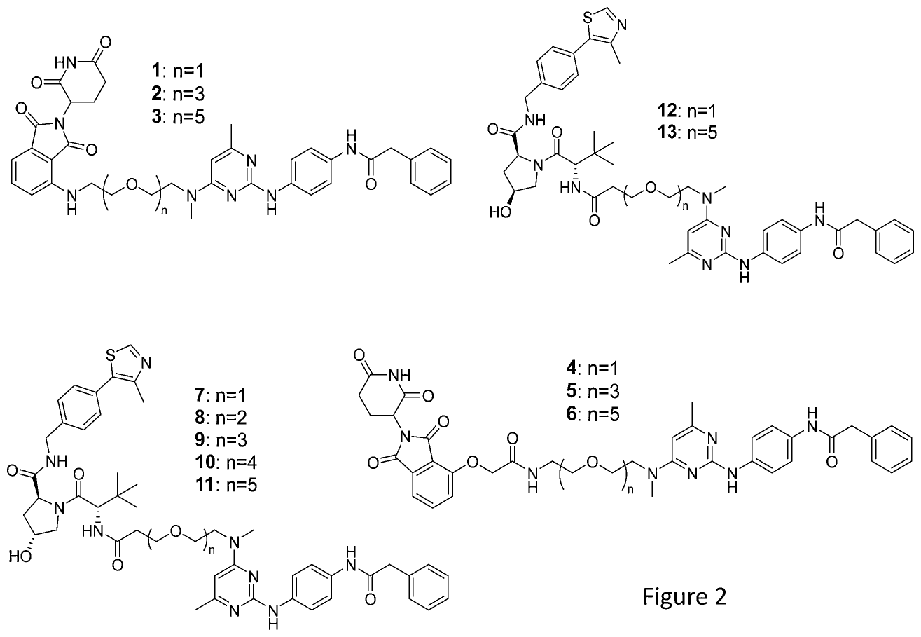

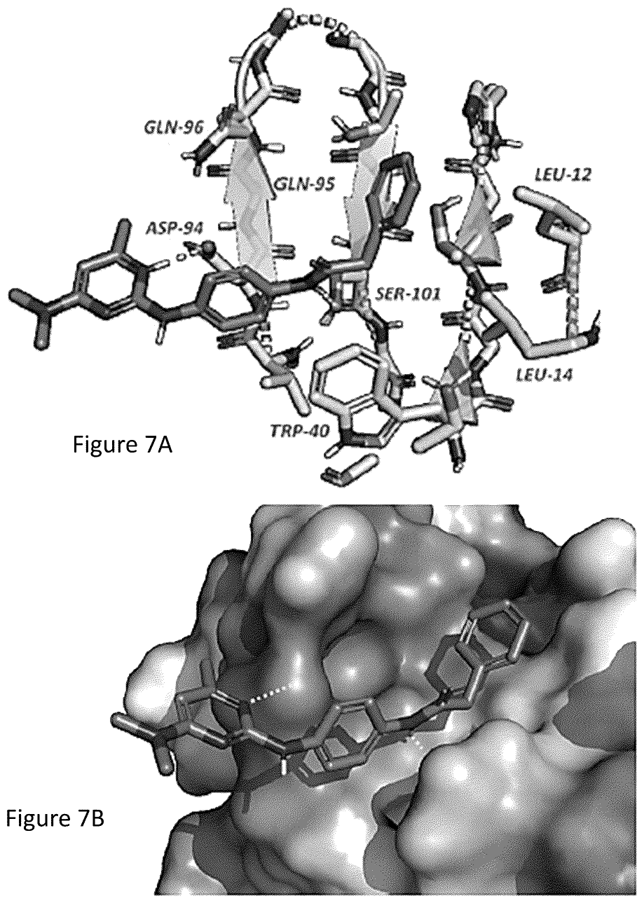

A small library of heterobifunctional derivatives was synthesized to assess their potential to degrade TG2 (FIG. 2). TG2 inhibitor (MT4, FIG. 1) was chosen for TG2-binding ligand,41 as this compound has better solubility than another compound (TG53, FIG. 1) that was published,⁹ it is more potent in cellular experiments than TG53,⁴¹ and the binding affinity to TG2 was previously characterized.⁴¹ To determine an initial attachment point for the linkers of the bifunctional compounds, MT4 was first docked into the fibronectin-binding site of the crystal structure of TG2 (PDB 4PYG) to gain an understanding of the most likely solvent-accessible site (FIGS. 7A-7B). Based on the docked pose, several potential interactions were identified that contribute to ligand binding, including a hydrogen bond between the amide N—H and Ser101 and a hydrogen bond between the protonated pyrimidine and Asp94 (FIG. 7A). The phenyl ring appears to be positioned in the interior of the protein in a hydrophobic region formed by Leu12, Leu14, and Trp40. A potential hydrogen bond between Thr42 and the benzyl group pi system may also be present. In contrast, the dimethylamino group appears to be positioned toward the solvent-accessible region of the binding site (FIG. 7B). Based on this, as well as the relatively straightforward nucleophilic aromatic substitution chemistry needed to install linker amines at the pyrimidine-4-position, an initial set of PROTACs with linkers coming off the amine group was prepared.

The compound set included CRBN-binding ligands attached via either amines or ethers, as well as a VHL ligand attached by its terminal amine. All compounds used poly(ethylene glycol) (PEG) linkers of varying lengths. The synthesis of the CRBN-based compounds began with reacting 2,4-dichloro-6-methyl-pyrimidine (14) with amino-PEGn=1-3 linkers to give 4-amine-linked derivatives 15-17 in 20-39% yields (Scheme 1). Following this amination, displacement of the chlorine in the 2-position of the pyrimidine ring was done using commercially available N-(4-aminophenyl)-2-phenyl-acetamide in the presence of acetic acid in isopropanol to afford 2,4-diaminopyrimidines¹⁸⁻²⁰ in good-to-moderate yields. The hydroxyl group was tosylated, followed by a reaction with sodium azide to give azides²⁴⁻²⁶ in good yields. Hydrogenation of the azide to amines²⁷⁻²⁹ followed by a nucleophilic aromatic substitution reaction with 2-(2,6-dioxopiperidin-3-yl)-4-fluoroisoindoline-1,3-dione gave final compounds 1-3. Alternatively, an amide coupling reaction between amines²⁷⁻²⁹ and 2-((2-(2,6-dioxopiperidin-3-yl)-1,3-dioxoisoindolin-4-yl)oxy)acetic acid gave compounds 4-6. For VHL-based PROTACs (Scheme 2), the commercially available methylamino-PEGn-t-butyl esters were reacted with 2,4-dichloro-6-methylpyrimidine (14) to give the PEGn=1-5 pyrimidines 30-34 in 32-82% yields. Nucleophilic aromatic displacement of the pyrimidines with N-(4-aminophenyl)-2-phenyl-acetamide in refluxing isopropanol delivered the 2,4-diamino-substituted intermediates 35-39. The t-butyl ester was deprotected using HCl to give the carboxylic acids (40-44) in good yield. This was followed by an amide coupling with the appropriate VHL ligand to give desired compounds 7-11 as shown in Scheme 2. To prepare the negative control versions of active degraders 7 and 11, in which the stereochemical configuration of the C4 center on the VHL ligand pyrrolidine ring was inverted, a 2-(7-azabenzotriazol-1-yl)-N,N,N′,N′-tetramethyluronium hexa-fluorophosphate (HATU)-promoted amide coupling of acids 40 and 44 was carried out with the commercially available S,S,S-isomer to give negative control compounds 12 and 13 in moderate yields.

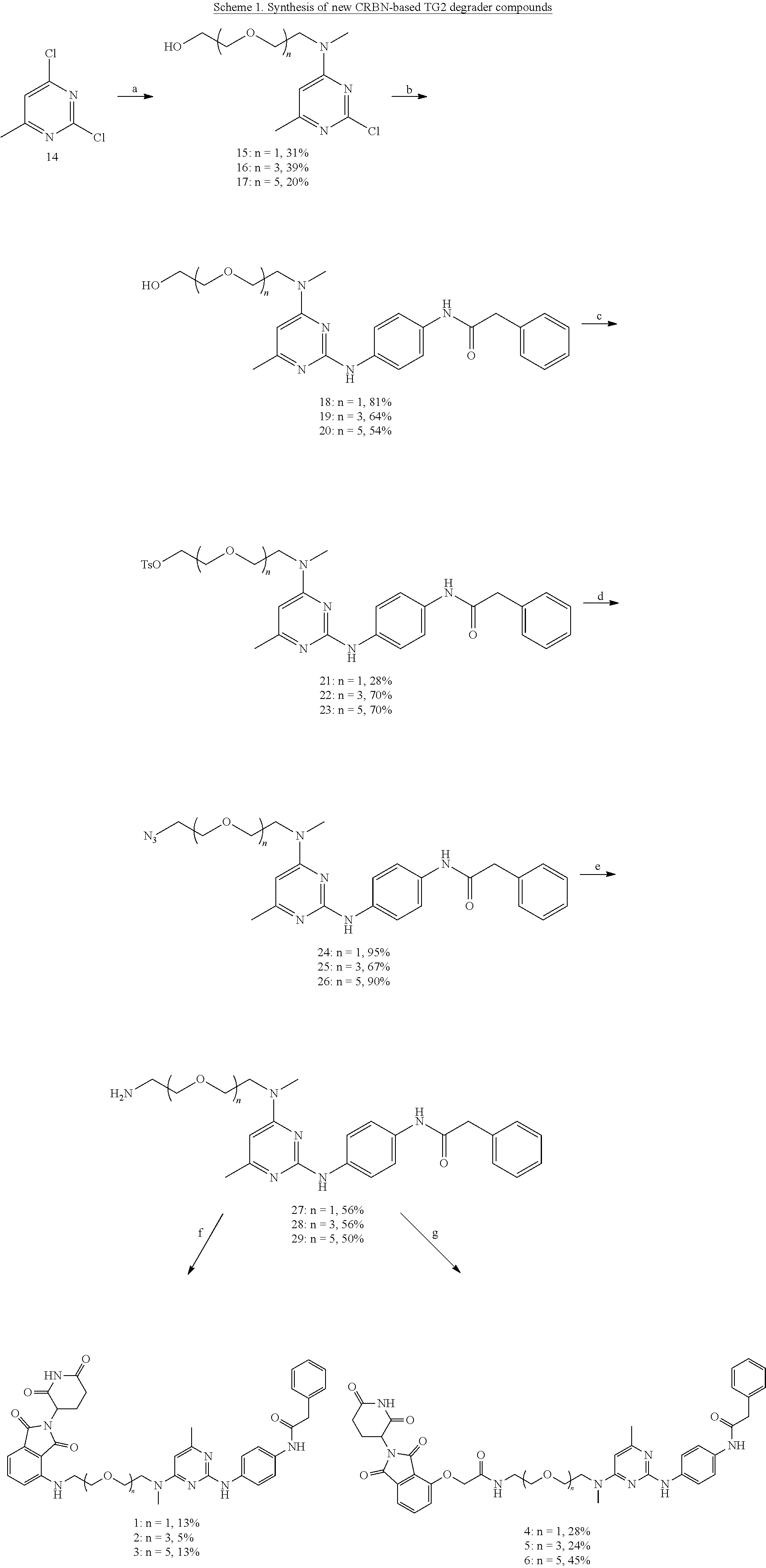

Reagents and conditions: (a) DIPEA, THF, methylamino-PEGₙ-OH, 20° C., 12 h; (b)N-(4-aminophenyl)-2-phenyl-acetamide, AcOH, IPA, 80° C., 12 h; (c) TosCl, TEA, DCM, 20° C., 8 h; (d) NaN₃, DMF, 50° C., 12 h; (e) H₂, Pd/C, NH₄OH, MeOH, 20° C., 12 h; (f) 2-(2,6-dioxopiperidin-3-yl)-4-fluoroisoindoline-1,3-dione, DIPEA, DMSO, 60° C., 12 h; (g) 2-((2-(2,6-dioxopiperidin-3-yl)-1,3-dioxoisoindolin-4-yl)oxy)acetic acid, HATU, DIPEA, DMF, 20° C., 12 h

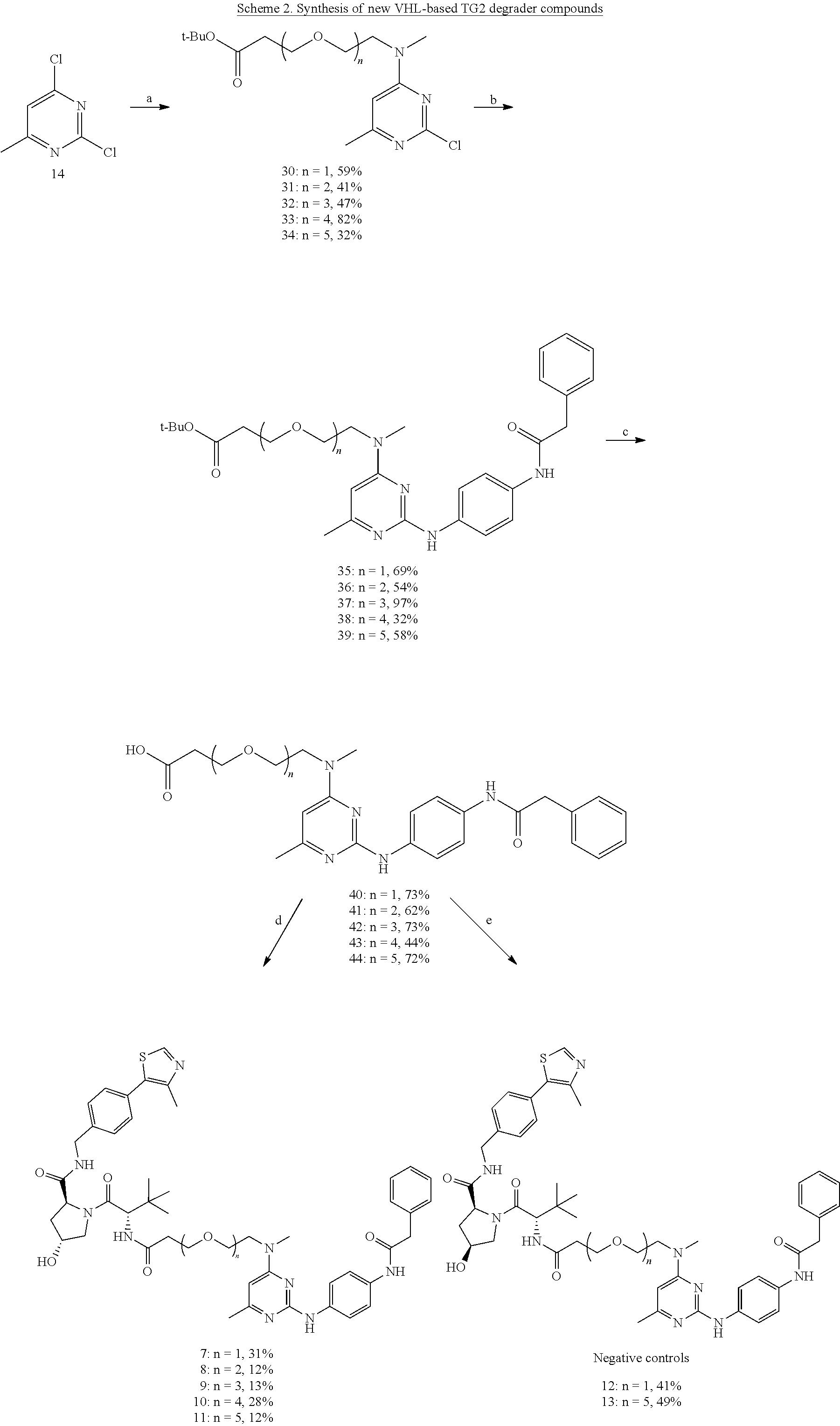

Reagents and conditions: (a) DIPEA, THF, methylamino-PEGₙ-t-butyl ester, 0° C. to 15° C., 10 h; (b)N-(4-aminophenyl)-2-phenyl-acetamide, AcOH, IPA, 80° C., 12 h; (c) HCl in ethyl acetate (4 M), 25° C., 12 h; (d) (S,R,S)-AHPC, HATU, DIPEA, DMF, 25° C., 12 h; (e) (S,S,S)-AHPC hydrochloride, HATU, DIPEA, DMF, 25° C., 12 h

### Screening for TG2 Degradation in Ovarian Cancer Cell Lines

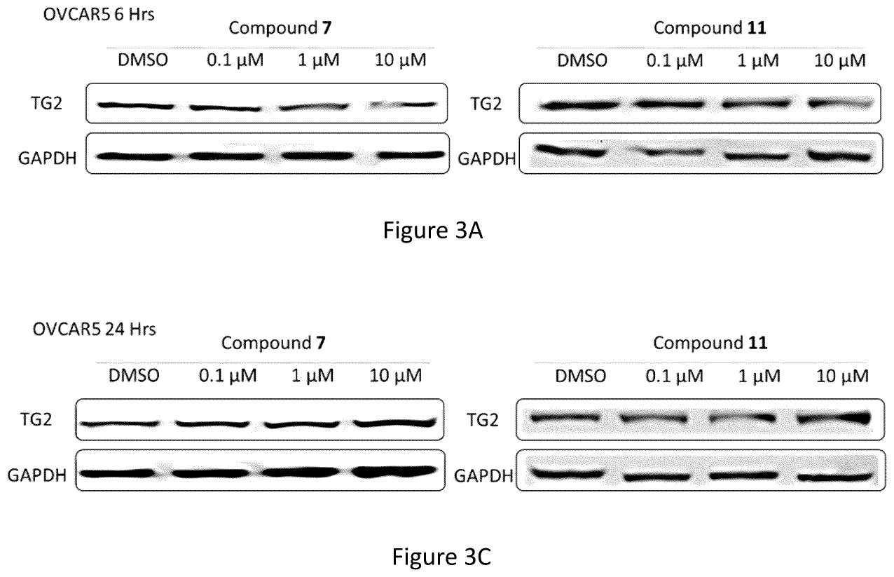

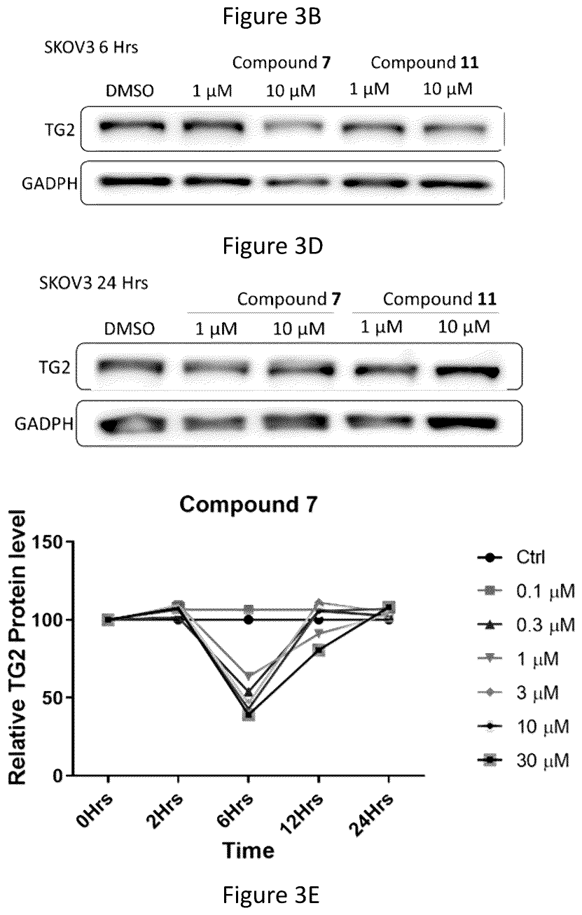

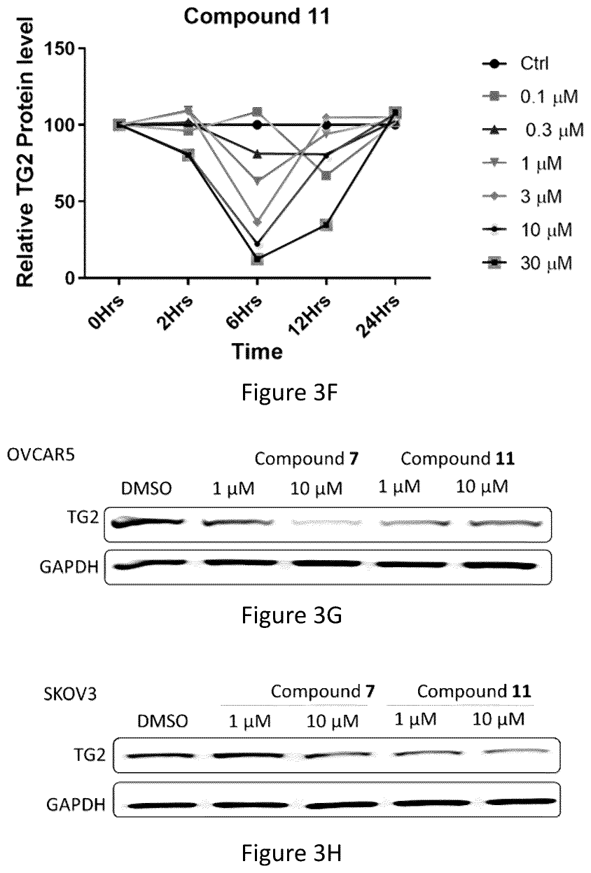

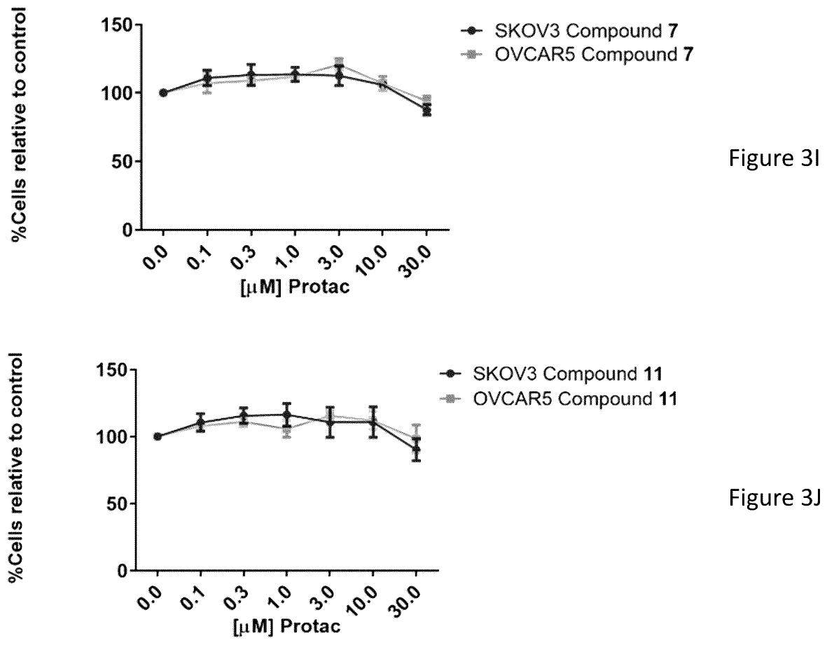

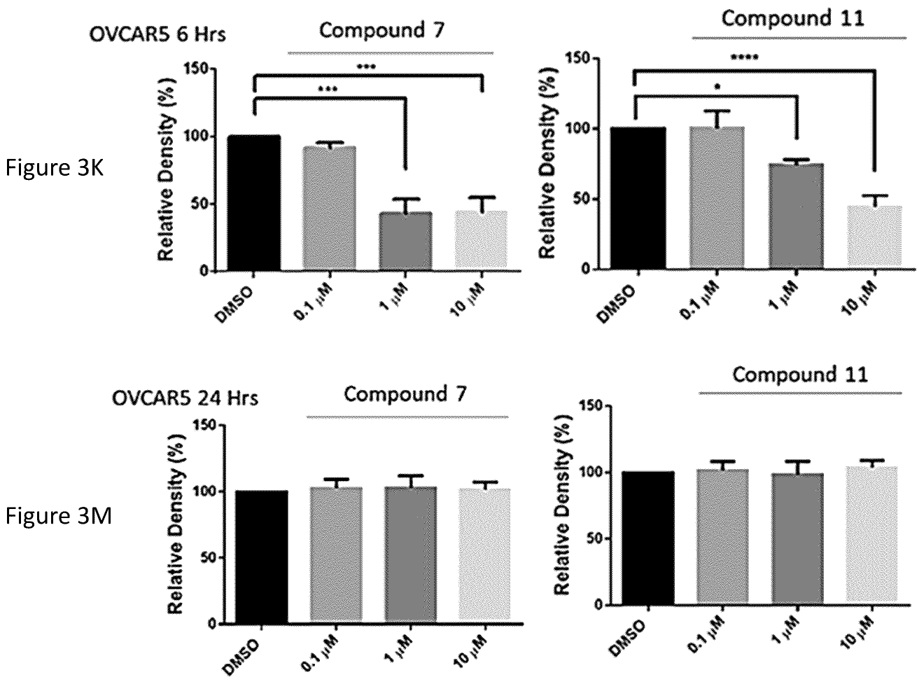

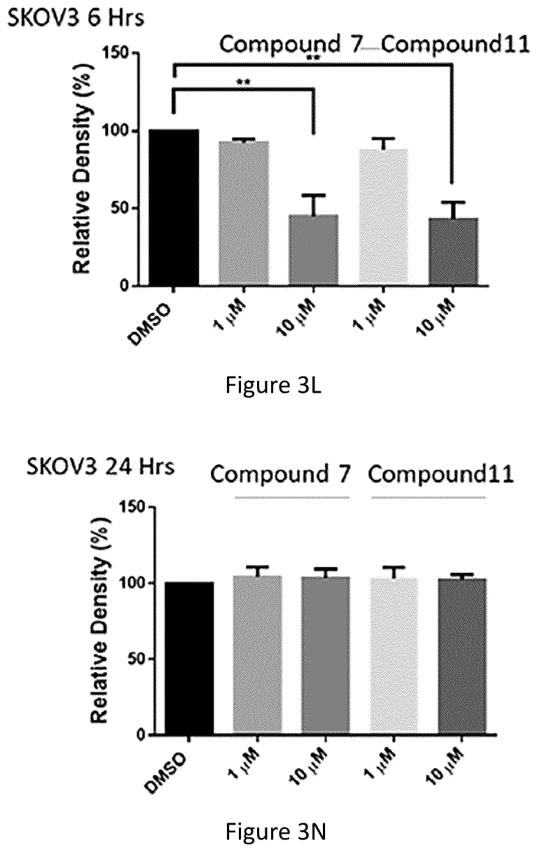

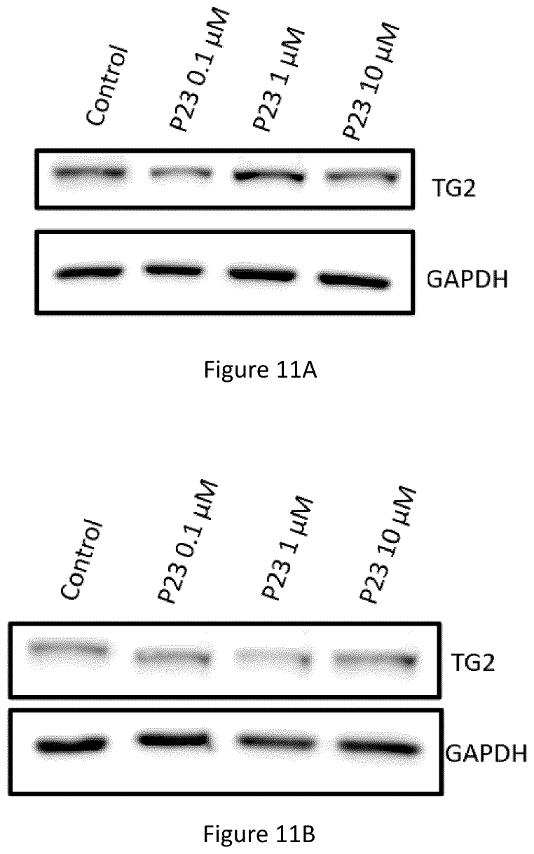

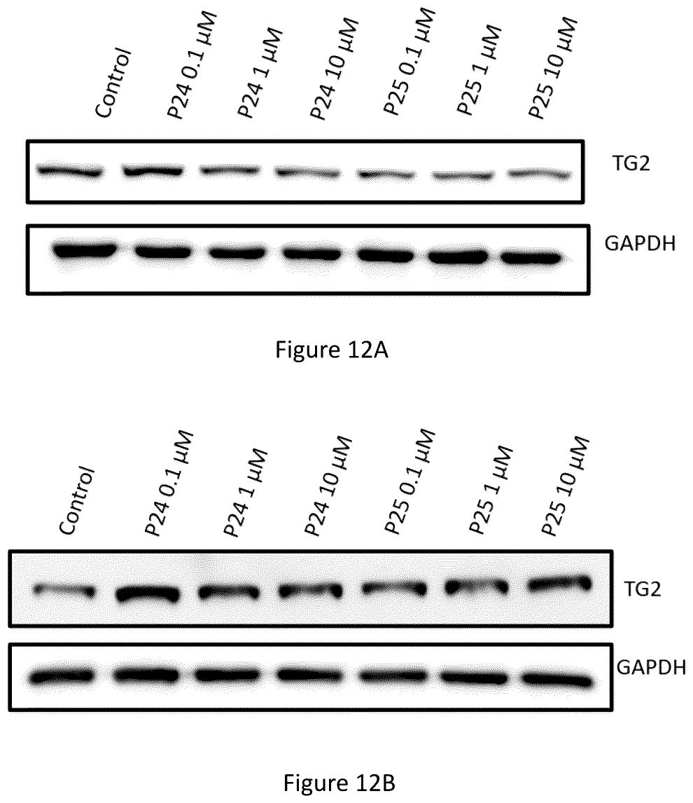

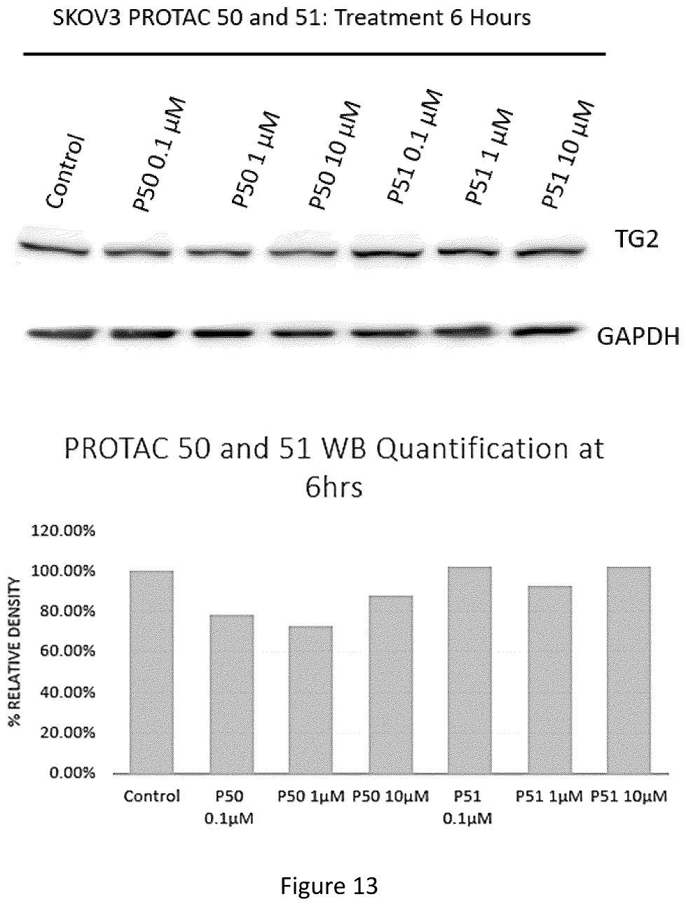

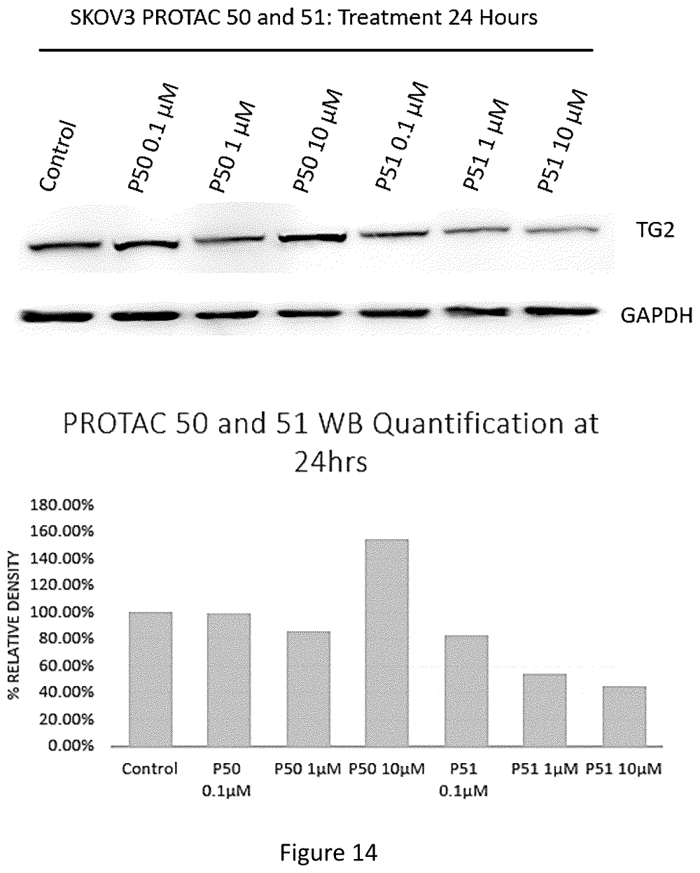

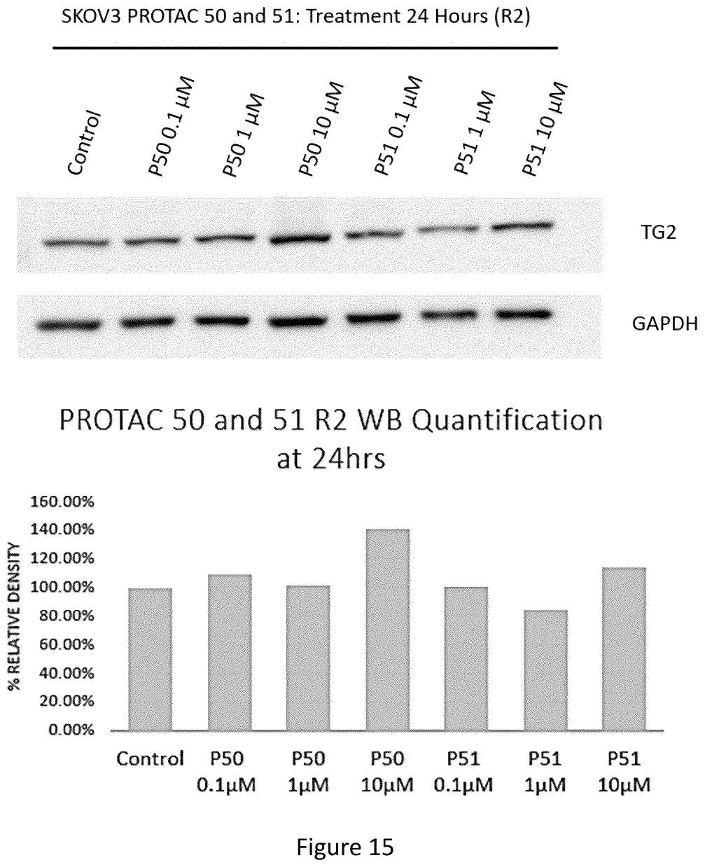

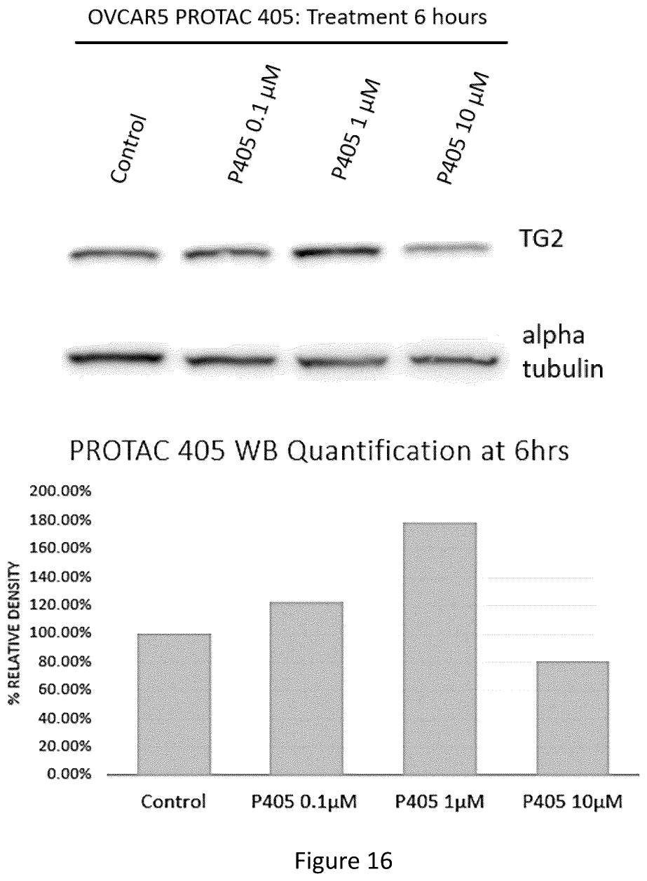

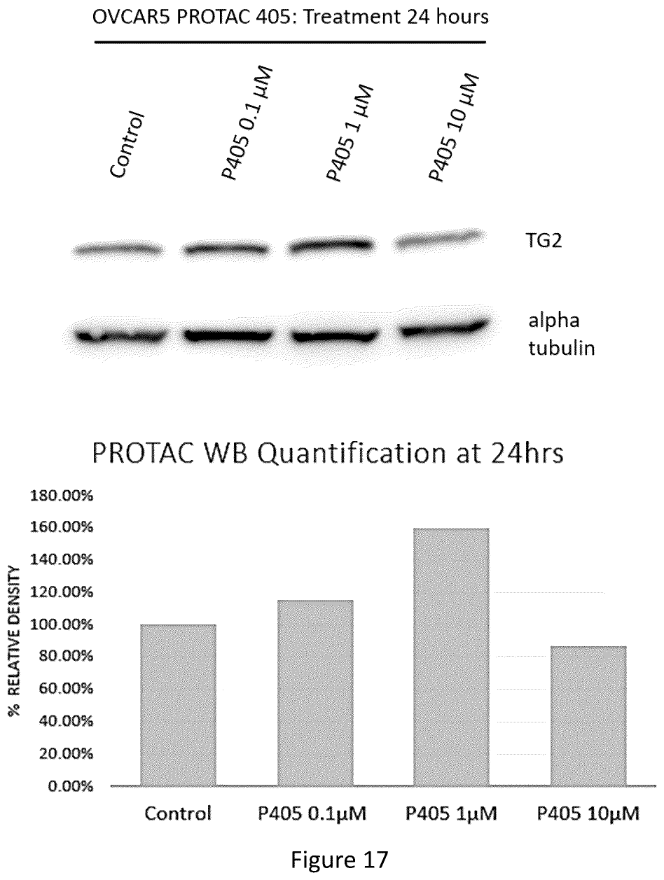

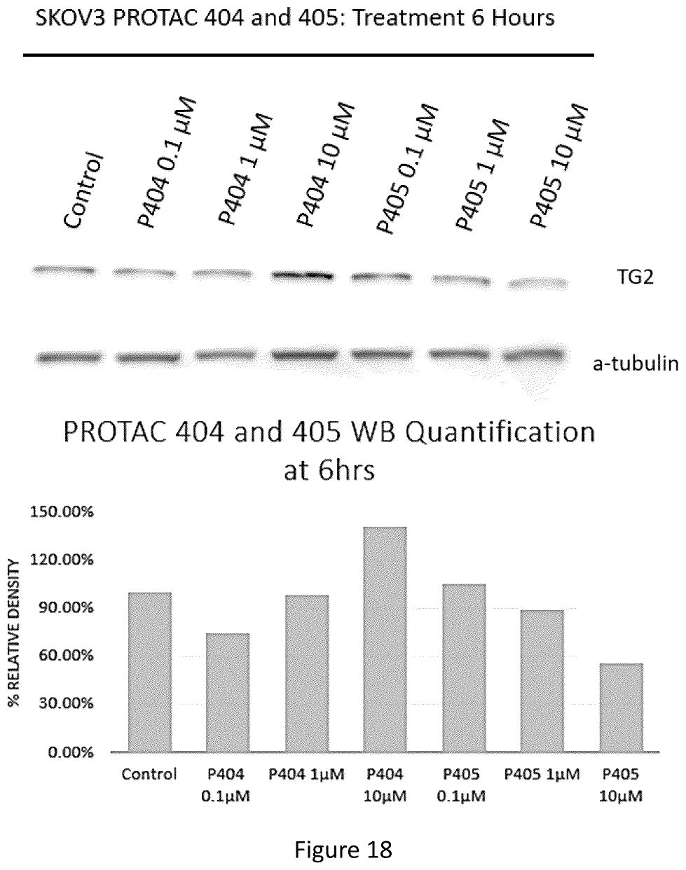

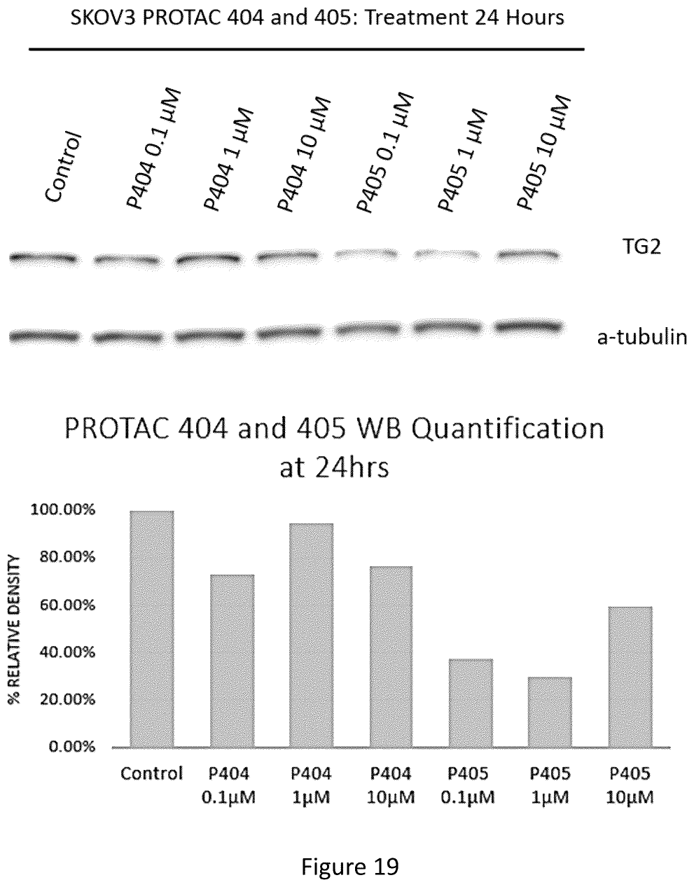

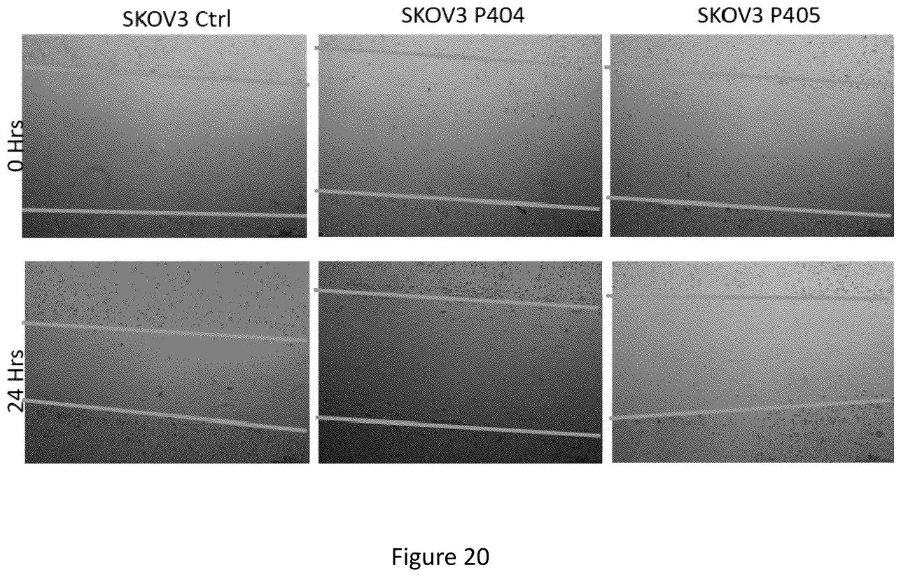

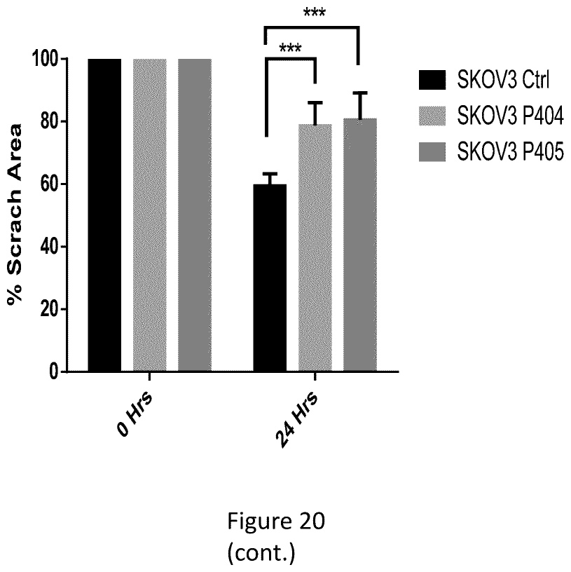

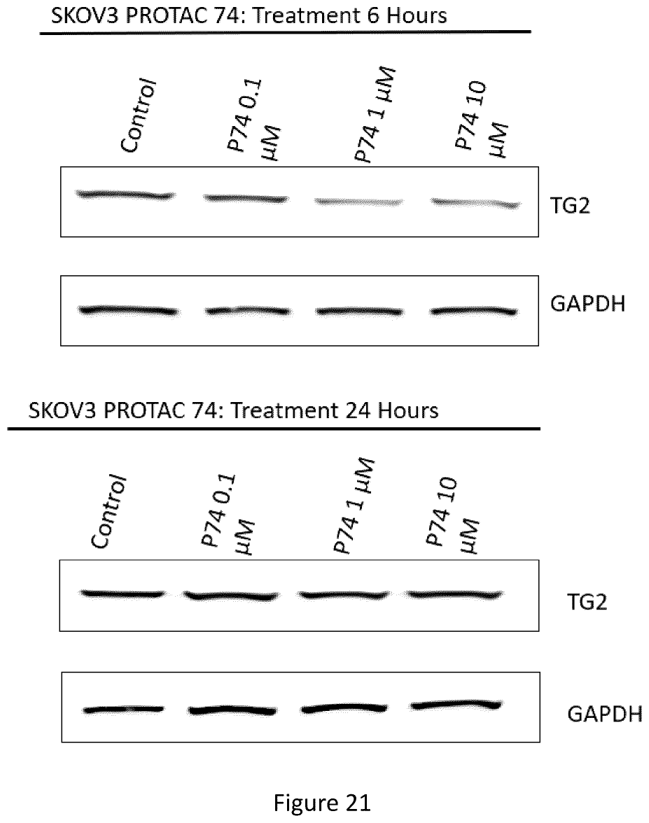

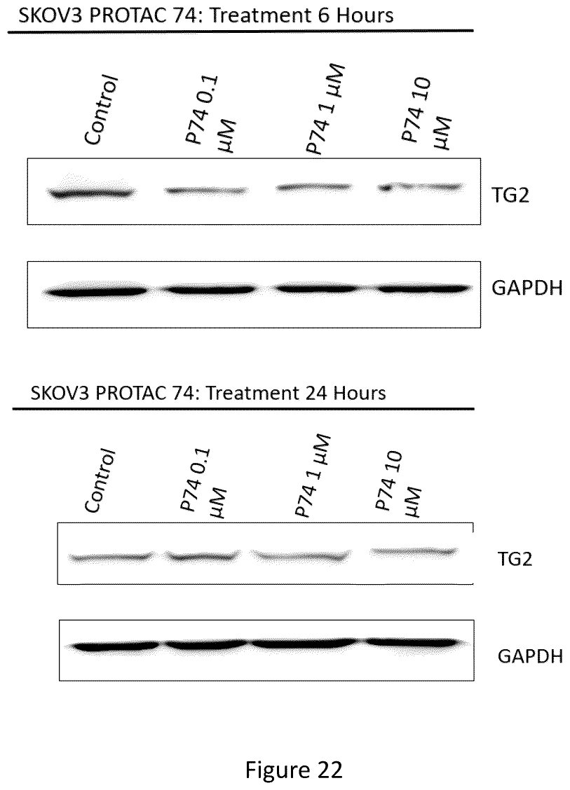

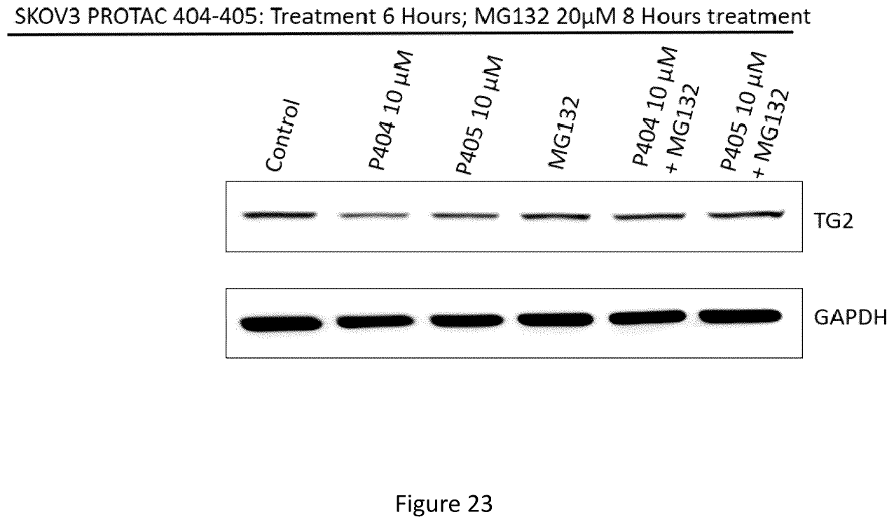

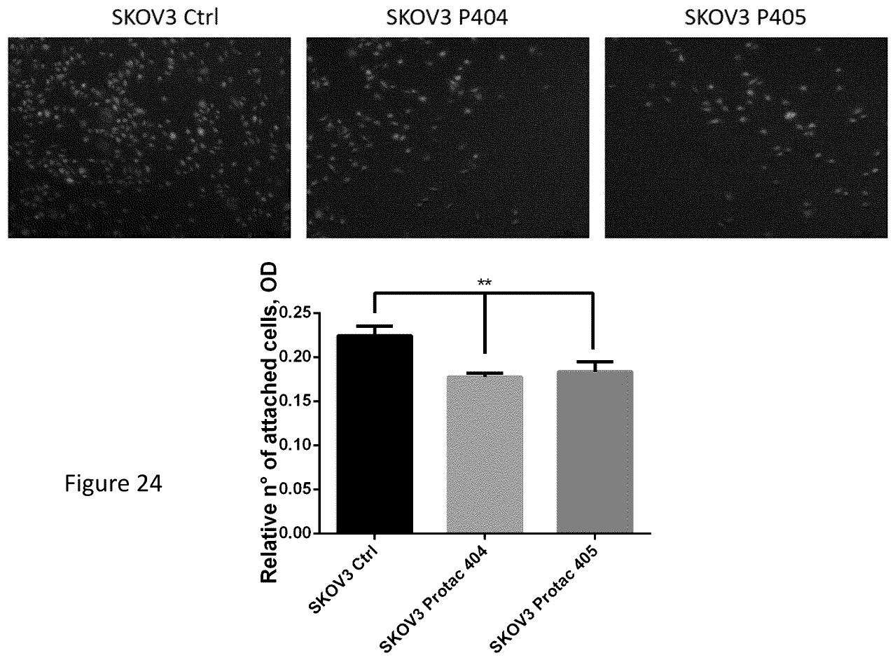

Whether the synthesized compounds induced TG2 degradation was tested in ovarian cancer cell lines OVCAR5 and SKOV3 using concentration range (0.1-10 μM) and time course (6-24 hours) experiments. The limited solubility of the compounds precluded their evaluation at concentrations above 10 μM. Among this set of compounds, 7 and 11 both significantly reduced the level of TG2 within 6 hours, in a concentration-dependent manner in both OVCAR5 and SKOV3 cells (FIGS. 3A, 3B, 3K, and 3L), whereas no TG2 degradation was obtained after treatment with the other compounds (data not included). However, longer (24 hour) treatment with compounds 7 and 11 did not induce reduction in TG2 levels (FIGS. 3C, 3D, 3 M, and 3N). To further study the dynamics of TG2 degradation induced by the active degraders, SKOV3 cells were treated with different concentrations of compounds 7 and 11 at different time points. Using western blotting analysis, a concentration-dependent response (0.1, 0.3, 1, 3, 10, 30 μM) after 2-, 4-, 6-, and 12-hours treatment with 7 (FIG. 3E) and 11 (FIG. 3F) was observed. Both compounds induced TG2 degradation in a time dependent manner, with maximal degradation observed at 6 hours using higher concentrations (10 and 30 μM). After this degradation maximum, the TG2 levels started to increase at 12 hours, returning to levels close to control at 24 hours. However, repeated dosing with compounds 7 or 11 over a 24 hour period (every 6-8 hours) resulted in persistent and dose-dependent decreased TG2 levels at 24 hours in both OVCAR5 and SKOV3 cells (FIGS. 3G and 3H). To exclude the possibility that the observed decreased TG2 levels induced by the degraders were related to cell death, viability assays were carried out. For this, OVCAR5 and SKOV3 cells were treated with compounds 7 and 11 every 6-8 h for a period of 5 days. No significant cell death was observed after treatment with any concentrations of compounds 7 (FIG. 3I) or 11 (FIG. 3J).

### Degradation of TG2 is Proteasome-Dependent

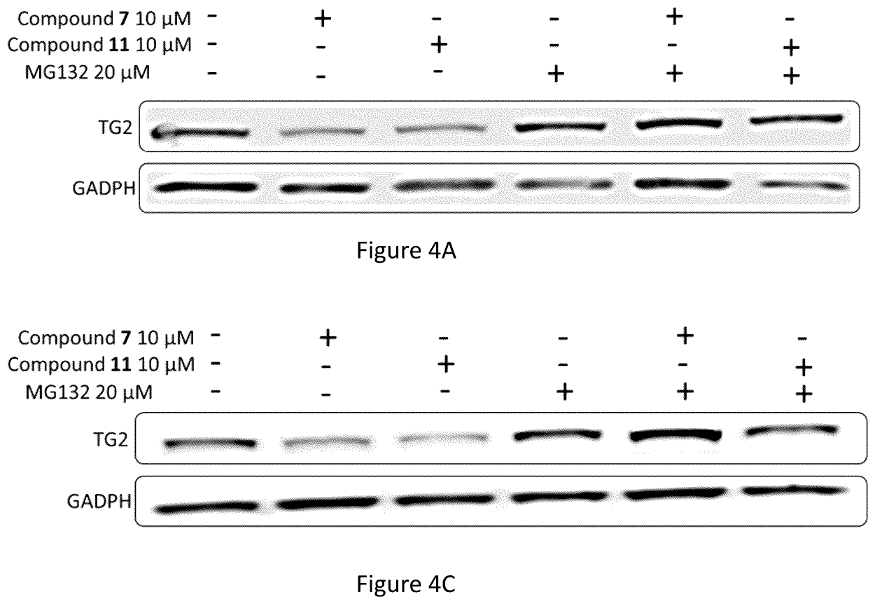

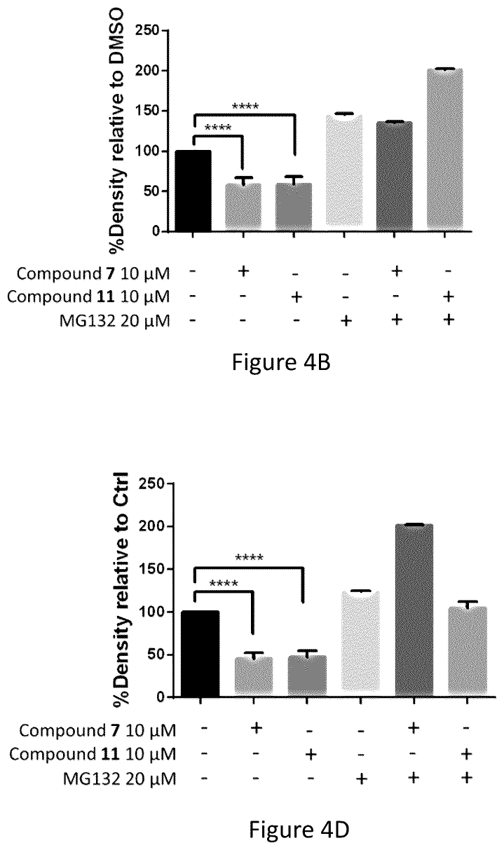

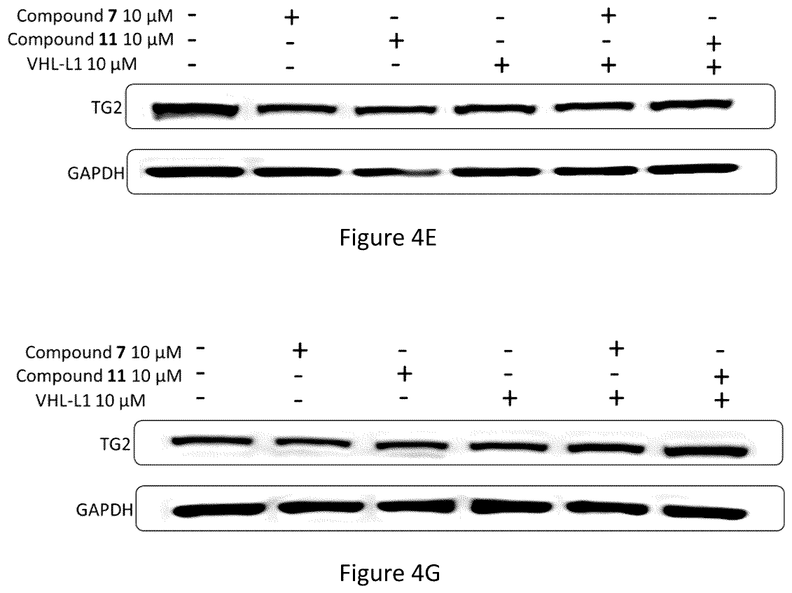

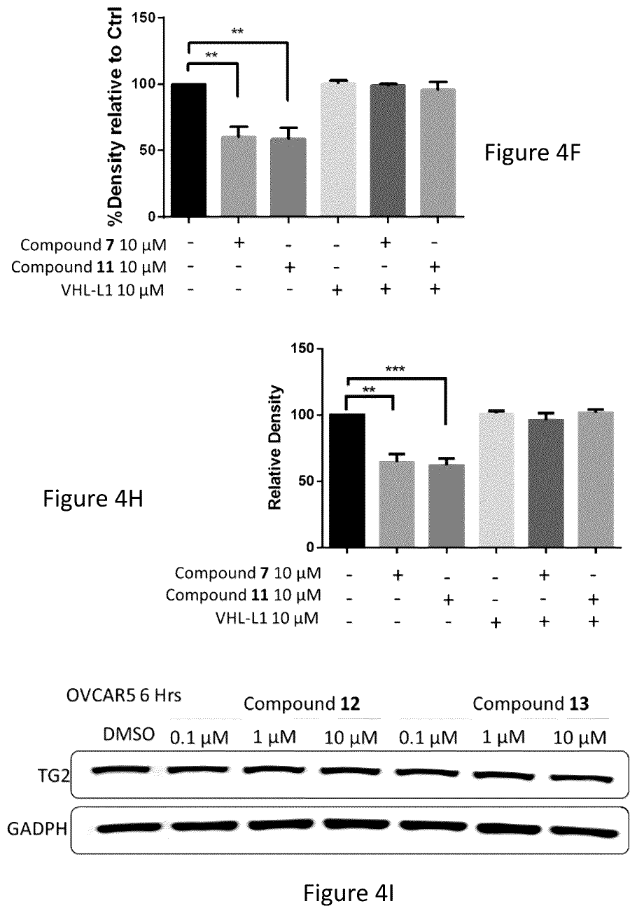

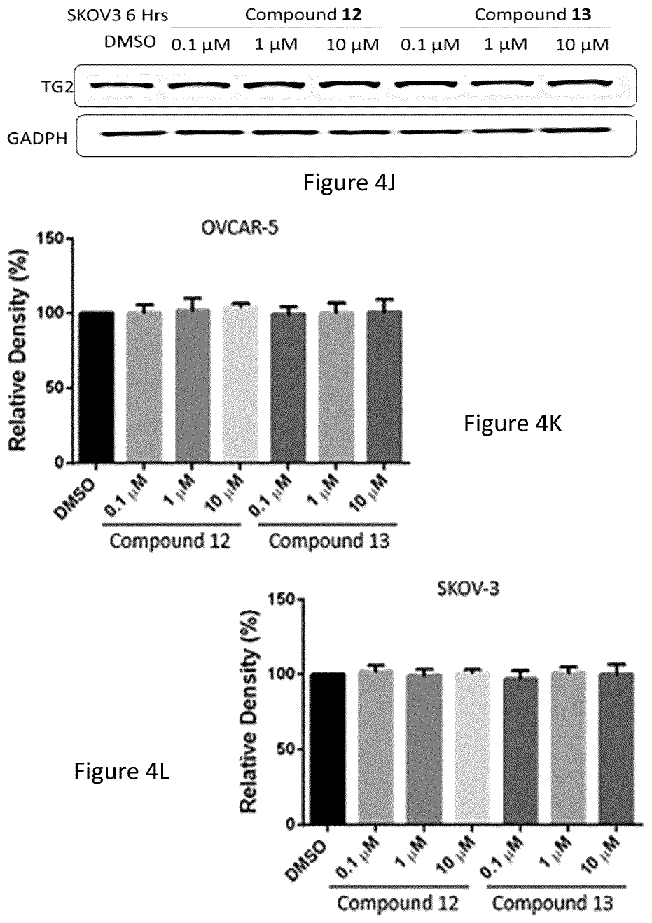

PROTACs act by engaging proteasome-dependent protein degradation mechanisms. To determine whether the compounds required proteasome activity for the degradation of TG2, their effects in the presence or absence of the proteasome inhibitor MG132 were tested. Treatment with MG132 prevented TG2 degradation in both OVCAR5 (FIGS. 4A and 4B) and SKOV3 (FIGS. 4C and 4D) cells, supporting the proteasome-dependent nature of TG2 degradation. To further confirm this mechanism, whether treatment with the von Hippel-Lindau (VHL) ligand (VHL-L) would compete with the bifunctional degraders for VHL binding was tested, thereby abrogating their effects. Indeed, treatment with VHL-L did not alter levels of TG2, while co-treatment of VHL-L with either 7 or 11 prevented degradation of TG2 in both OVCAR5 (FIGS. 4E and 4F) and SKOV3 (FIGS. 4G and 4H) cells. To further establish that degradation by compounds 7 and 11 is proteasome-dependent, negative control versions of these degraders were prepared. These compounds, 12 and 13 (Scheme 2), have inverted configurations of the C4 stereocenter of at the pyrrolidine ring and are unable to bind VHL, and therefore do not induce proteasome-dependent degradation. Treatment of OVCAR-5 cells (FIGS. 4I and 4K) or SKOV3 cells (FIGS. 4J and 4L) with 12 and 13 for 6 hours did not induce TG2 degradation at either 0.1, 1, or 10 μM in three separate replicates, demonstrating that recruitment of the VHL E3 ligase complex by 7 and 11 is necessary for the degradation of TG2.

### Degradation of TG2 Inhibits Ovarian Cancer Cell Migration

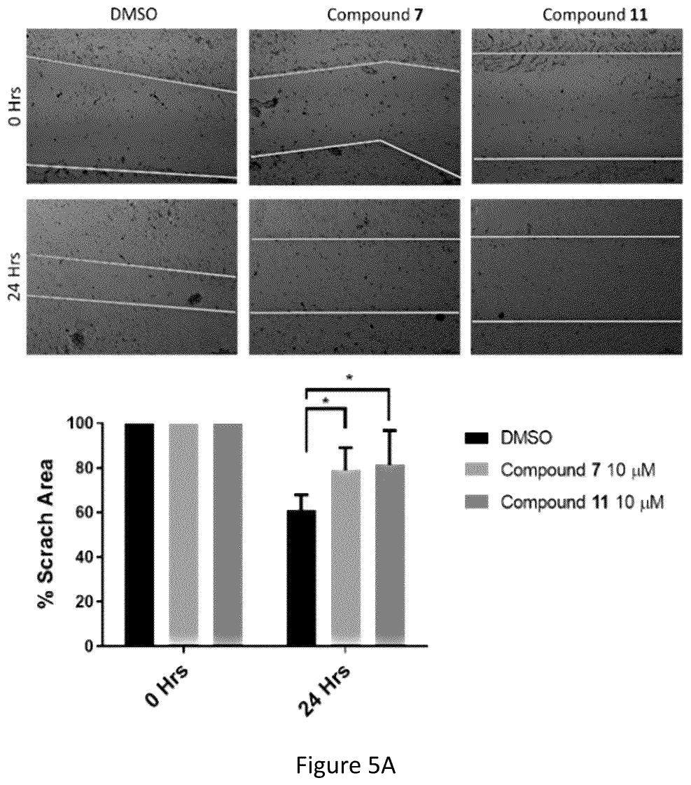

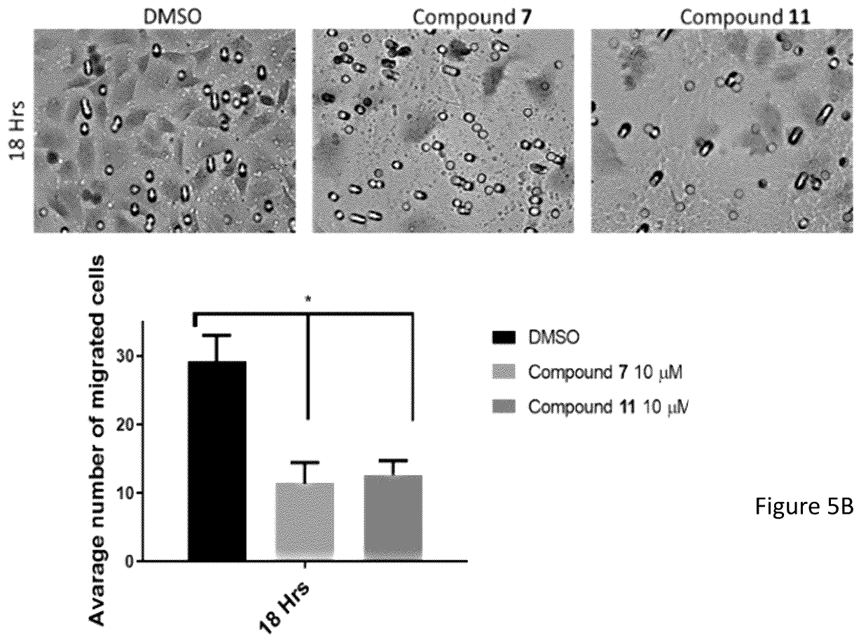

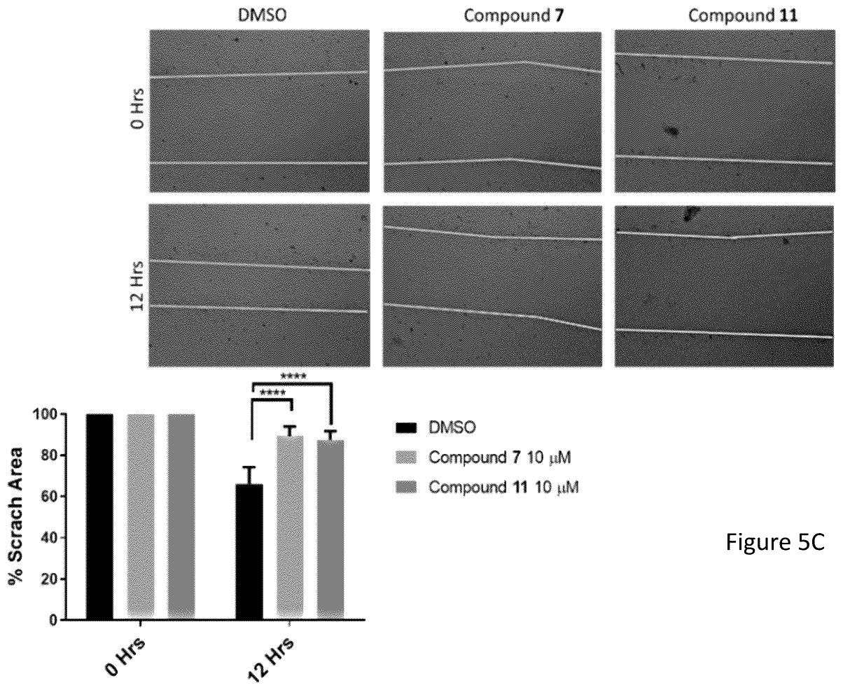

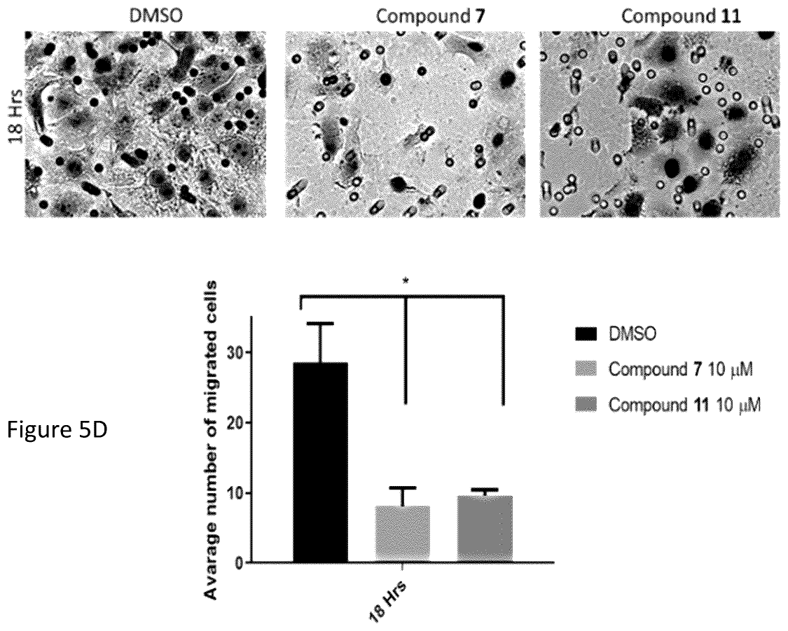

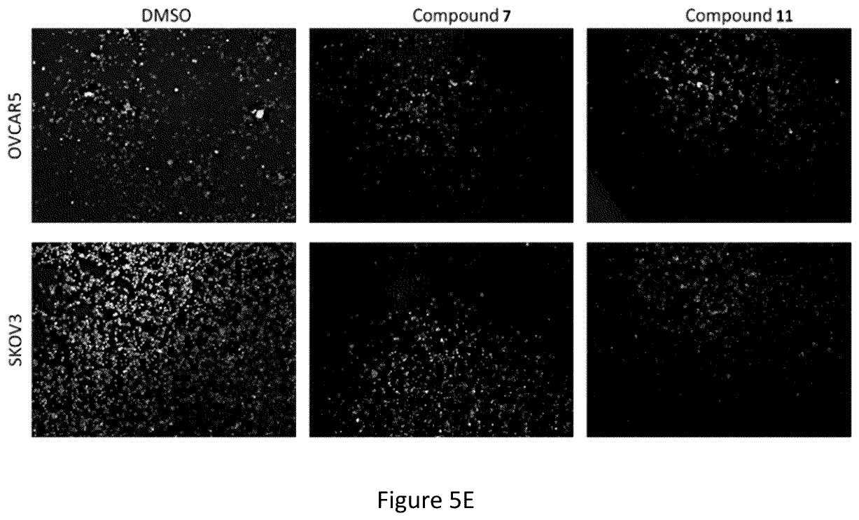

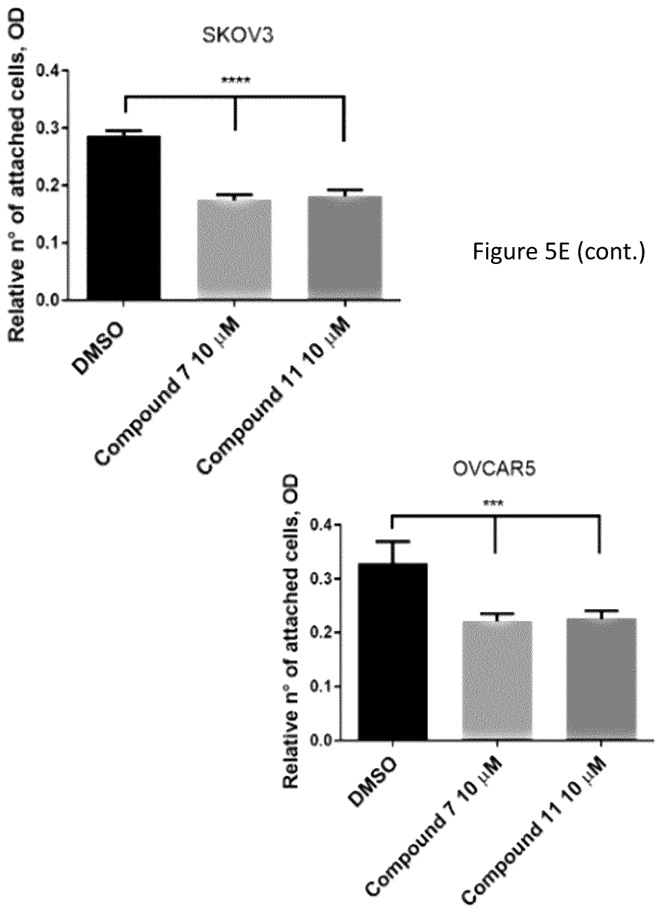

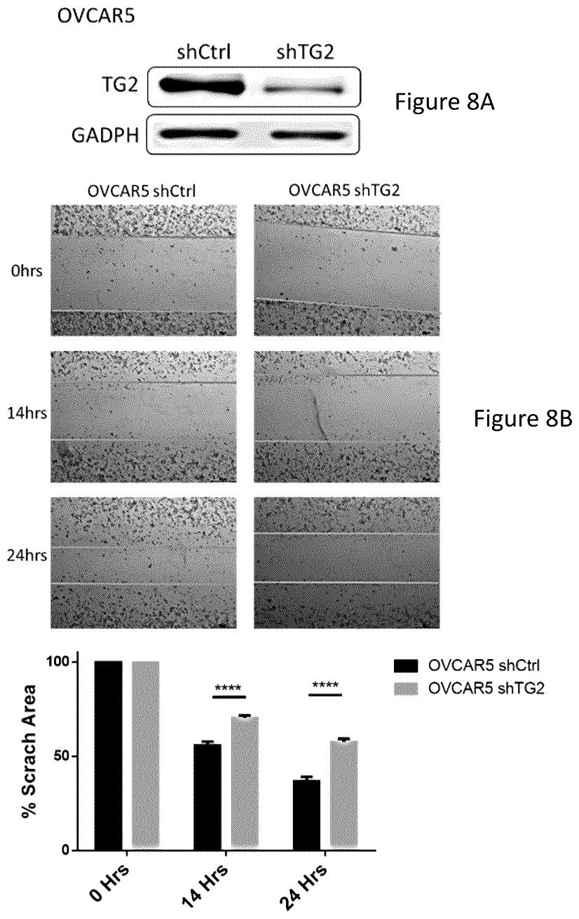

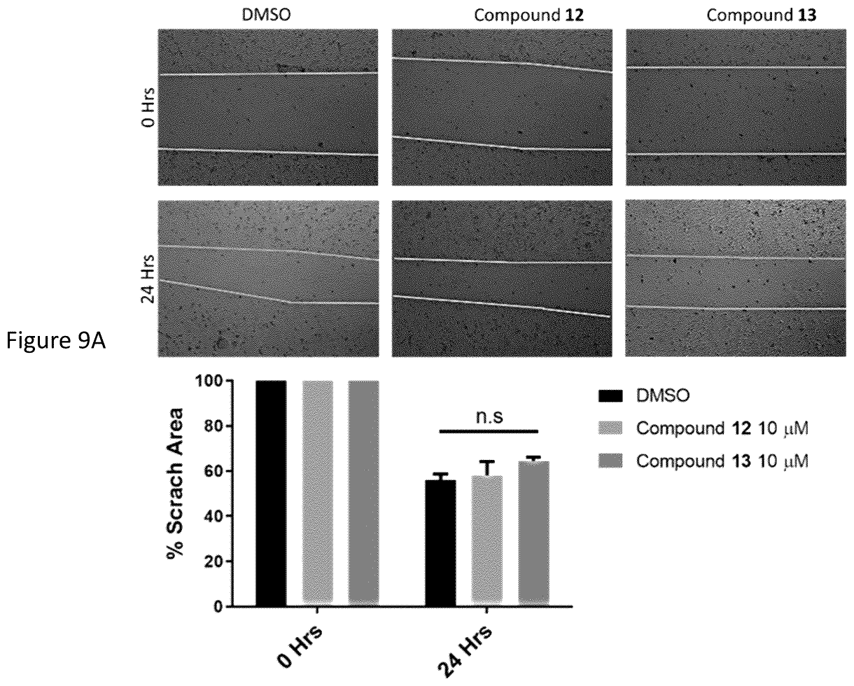

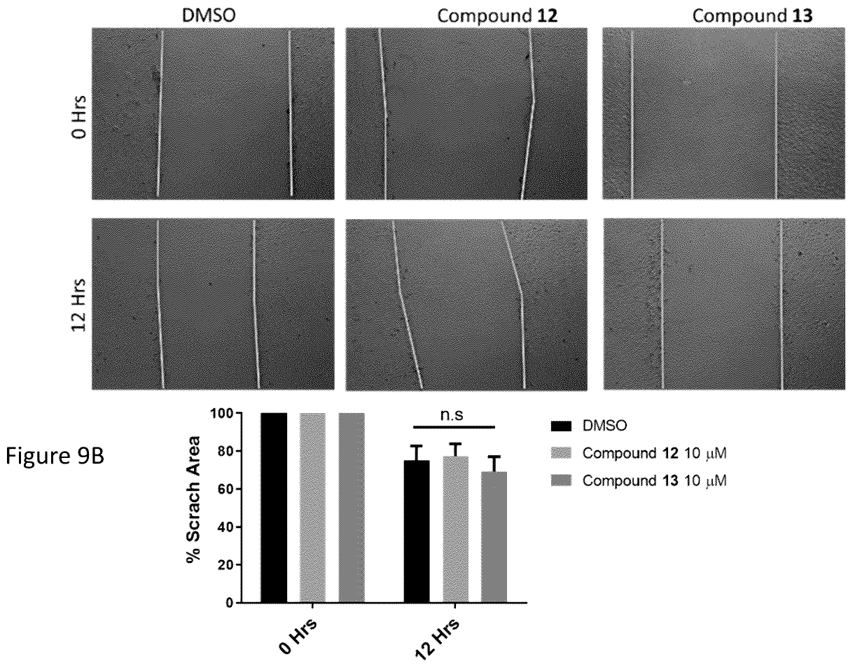

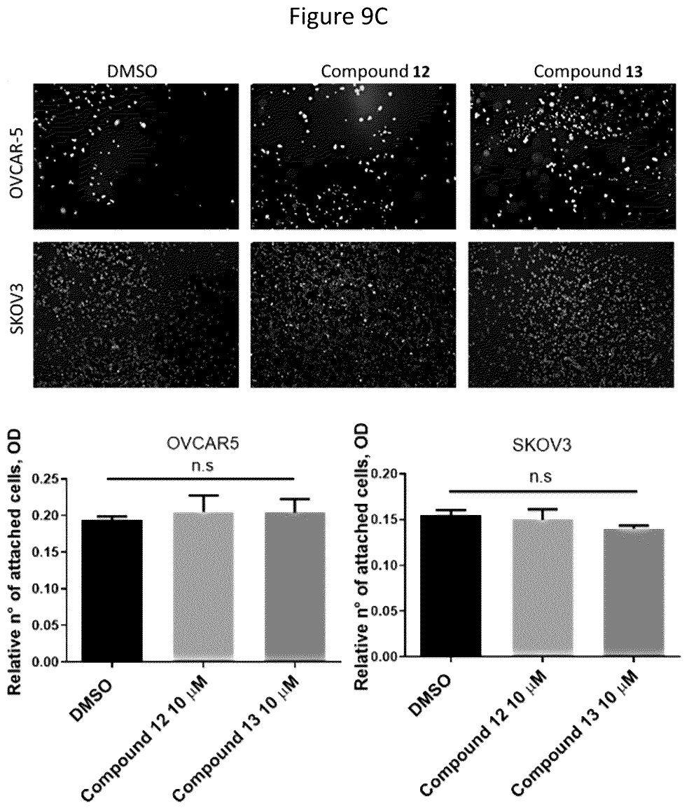

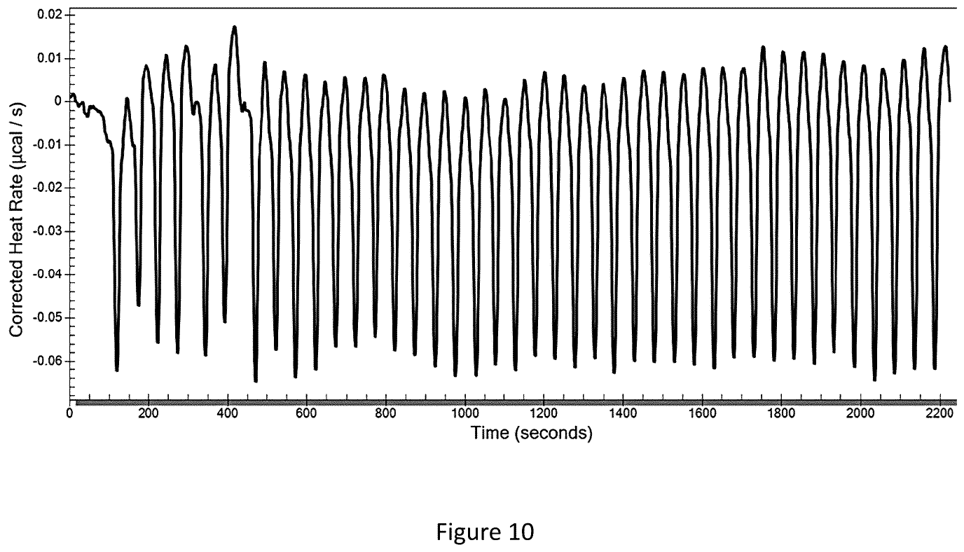

After compounds 7 and 11 were demonstrated to effectively degrade TG2 in OC cell lines, their functional effects were tested. It has been established that the genetic knockdown of TG2 using shRNAs reduces the migration and invasion of cancer cells by blocking TG2-induced EMT.²⁰,4¹ TG2 knockdown in OVCAR5 cells decreased cell migration was confirmed, as measured through a scratch assay (FIGS. 8A-8B), supporting the role of TG2 in cell motility. The effects of the TG2 degraders on cell migration and invasion were therefore tested by using wound-healing and transwell migration assays. Degraders 7 and 11 each potently inhibited the migration of OVCAR5 cells in both wound-healing assays (FIG. 5A, p<0.05) and transwell migration assays (FIG. 5B, p<0.05). It was found that compounds 7 and 11 similarly inhibited the migration of SKOV3 cells (FIG. 5C, p<0.05 and FIG. 5D, p<0.05) in wound-healing and transwell migration assays, respectively. As TG2 is involved in the adhesion of cells on fibronectin,⁴¹,7² the effects of the TG2 degraders on the adhesion of OVCAR5 and SKOV3 cells to fibronectin were also tested. Treatment with 7 and 11 significantly reduced the adhesion of cells to fibronectin (FIG. 5E, p<0.05). On the other hand, treatment of both OVCAR5 and SKOV3 cells with inactive degraders 12 and 13 did not affect cell migration (FIGS. 9A,B) and adhesion (FIG. 9C). Altogether, these results indicate that targeted protein degradation of TG2 has significant functional effects on the phenotype of ovarian cancer cells, affecting migration, invasion, and cell adhesion to the matrix.

### MT4 and PROTAC Bind to TG2

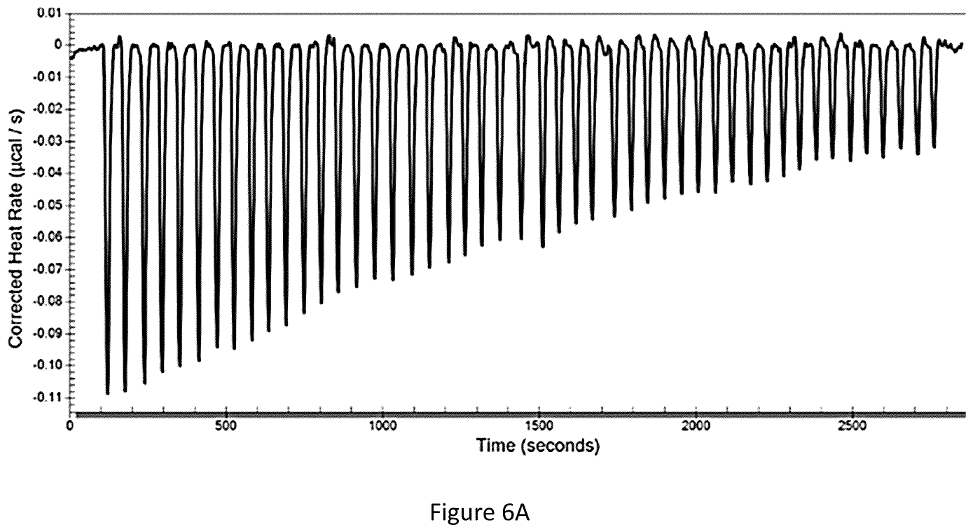

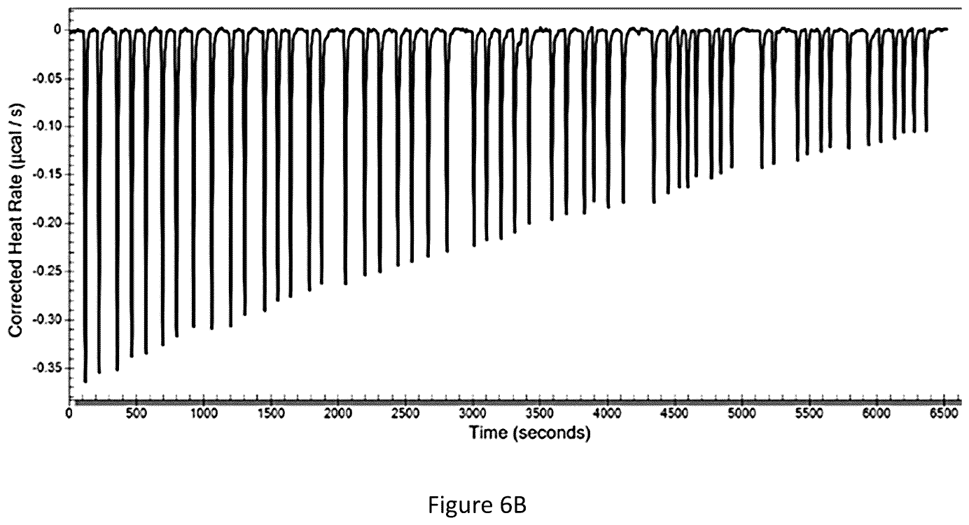

To confirm the direct binding of the PROTAC with TG2 and evaluate the effect of modifying the inhibitor into a bifunctional degrader on affinity, binding studies were carried out using isothermal calorimetry (ITC). The affinity of the parental inhibitor, MT4, was measured toward full-length TG2.⁴⁰ Titration of MT4 into a solution of TG2 showed an equilibrium dissociation constant KD=7.8 μM (FIG. 6A), which is in close agreement with the result for this compound (5.1 μM).⁴¹ The binding affinity of the two active PROTACs 7 and 11 to TG2 were then tested. Compound 11, with a PEG5 linker, showed a KD=68.9 μM, which was a decrease of 8.8-fold relative to the parental compound (FIG. 6B). Compound 7 did not show a significant dose-dependent saturation under these experimental conditions, indicating its KD>100 μM or a >12.8-fold decrease in affinity (FIG. 9).

### CONCLUSION

Over the last 10-15 years, there has been increasing awareness that TG2 plays a key role in a variety of tumorigenic and metastatic processes. It has been discovered that TG2 exerts several functions that drive pro-tumorigenic activity and has therefore been an attractive target for drug discovery.

However, in spite of significant efforts, no clinically effective TG2-targeting small molecules have been developed to date for cancer models. Given the multitude of functions exerted by this complex protein, it is likely that disrupting only one particular domain will be insufficient to provide efficacy. Because of the limited ability of traditional small-molecule inhibitors to target multiple protein functions simultaneously, a PROTAC strategy was initiated to target TG2. One of the key applications of targeted degradation approaches is for proteins that possess multiple functions or may serve as scaffold proteins. These targets have traditionally been very difficult to address with small-molecule enzymatic or protein-protein inhibitors. Degrading the TG2 protein may serve to abolish its myriad functions and thereby provide more robust and meaningful efficacy in cancers where TG2 plays a key role, such as ovarian cancer.

To test this approach, previously described inhibitor scaffold was used as the PROTAC ligand for the protein of interest.⁴⁰⁻⁴¹ This compound was attached via PEG linkers of varying lengths to known ligands for CRBN and VHL E3 ligases. This set of compounds was then tested in multiple ovarian cancer cell lines that express TG2 for their ability to degrade the protein. Two VHL-containing bifunctional degraders were found to induce the robust reduction of the TG2 protein in multiple ovarian cancer cell lines. Mechanism of action studies using co-treatment with the proteasome inhibitor MG132 or with the excess VHL ligand confirmed the proteasome-dependent nature of degradation. Negative control versions of the active degraders that were unable to bind VHL were also synthesized and they were found to fail to degrade TG2, further confirming the E3 ligase-dependent nature of degradation. The kinetics of degradation were further evaluated and it was found that the maximum reduction of TG2 occurred at 6 h, with recovery by 24 h. Importantly, in SKOV3 and OVCAR5 cells, the proposed TG2 PROTACs significantly reduced in vitro migration as assessed by both wound-healing and scratch assays. These effects were not seen with PROTAC-negative control compounds. These “chemical knockdown” experiments excitingly confirmed that TG2 is necessary for the migration of ovarian cancer cells and supported the results of previous studies using genetic methods to deplete TG2.³¹

To confirm the direct binding of the PROTACs with TG2 and further correlate the functional activity of the degraders to their binding and degradation of TG2, binding studies using ITC were carried out. Encouragingly, the results with the parental TG2 inhibitor MT4 were very close to the value recently reported.⁴¹ The affinity of PROTAC 11 with TG2 showed a clear binding effect, and the KD was measured to be 68.9 μM, which is an 8.8-fold decrease in affinity versus MT4. The decrease in the affinity of the PROTAC relative to the parental inhibitor is not unexpected, as many others have observed this as well, including with BRD4,⁷⁰⁻⁷³ where they found a 7.8-fold decrease in affinity for the PROTAC versus the inhibitor for BRD4-BD1, as well as when looking at PROTACs for a large set of kinases.⁴³ This ITC result demonstrates that PROTAC 11 binds TG2 and further supports the proposed proteasome-mediated (i.e., PROTAC) mechanism. This result is also consistent with data presented for inactive control compound 13, which failed to induce TG2 degradation or ovarian cancer cell migration. Data for degrader 7 did not show a binding effect under the experimental conditions used, which could only show binding affinity up to a KD<100 μM,⁷⁴ suggesting that this compound's affinity is >100 μM. A strong sensitivity of the TG2 protein to DMSO concentration was also observed, which limited the use of DMSO to 1%. The ITC data for 7 could also reflect this compound's poorer solubility than compound 11 in low DMSO concentrations, as it has a single PEG group in the linker, while 11 has a PEG5 unit, which presumably gives it better solubility.

The TG2 PROTACs described here are the first known targeted degraders of TG2 and provide a unique and useful set of molecular tools to study the functions of this important protein. Importantly, the reported approach uses a series of TG2-targeting compounds as the PROTAC ligands that are assumed to bind to the fibronectin-binding site. This site is separate from the transamidase enzymatic site, which is conformationally regulated by the presence of Ca²⁺ and GTP/GDP binding. By targeting the less-dynamic FN-binding site, the PROTACs are expected to be capable of degrading TG2 under a wider range of conditions. While the results show the dependency of TG2 on migratory phenotypes, further work to optimize their potency, efficacy, and pharmaceutical properties will be necessary to develop them into compounds usable in in vivo cancer models. Work to modify the TG2-binding moiety, the E3 ligase ligand chemistry, and linker composition is ongoing with the goal of optimizing agents to help further define the role of TG2 in disease and potentially as a new therapeutic strategy for a variety of cancers.

### Experimental Section

### General Chemical Methods.

All chemical reagents were obtained from commercial suppliers and used without further purification unless otherwise stated. Anhydrous solvents were purchased from Sigma-Aldrich, and dried over 3 Å molecular sieves when necessary. Normal-phase flash column chromatography was performed using Biotage KP-Sil 50 μm silica gel columns and ACS grade solvents on a Biotage Isolera flash purification system. Reverse phase prep conditions are detailed for each compound in the experimental write up. Analytical thin layer chromatography (TLC) was performed on EM Reagent 0.25 mm silica gel 60 F254 plates and visualized by UV light. Proton (¹H), and carbon (¹³C) NMR spectra were recorded on a 500 MHz Bruker Avance III with direct cryoprobe spectrometer. Chemical shifts were reported in ppm (δ) and were referenced using residual nondeuterated solvent as an internal standard (CDCl₃ at 7.24 ppm for ¹H-NMR and 77.0 for ¹³C-NMR; CD₃OD at 3.33 ppm for ¹H-NMR and 47.6 for ¹³C-NMR; DMSO-d₆ at 2.52 ppm for ¹H-NMR and 39.9 ppm for ¹³C-NMR). Proton coupling constants are expressed in hertz (Hz). The following abbreviations were used to denote spin multiplicity for proton NMR: s=singlet, d=doublet, t=triplet, q=quartet, m=multiplet, brs=broad singlet, dd=doublet of doublets, dt=doublet of triplets, quin=quintet, tt=triplet of triplets. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water+0.1% formic acid/5% acetonitrile+0.1% formic acid to 95% acetonitrile+0.1% formic acid/5% water+0.1% formic acid over 2 min plus a further minute continuing this mixture at a flow rate of 0.85 mL/min was used as the eluent. Total ion current traces were obtained for electrospray positive and negative ionization (ESI+/ESI−). Reactions were monitored via the integration of the appropriate UV traces, which corresponded to the desired mass of the product. High-resolution mass spectra were obtained using an Agilent 6210 LC-TOF spectrometer in the positive ion mode using electrospray ionization with an Agilent G1312A HPLC pump and an Agilent G1367B autoinjector at the Integrated Molecular Structure Education and Research Center (IMSERC), Northwestern University. All final compounds tested in biological assays are >95% pure by HPLC analysis.

### N-(4-((4-((2-(2-((2-(2,6-dioxopiperidin-3-yl)-1,3-dioxoisoindolin-4-yl)amino)ethoxy)ethyl)(methyl)amino)-6-methylpyrimidin-2-yl)amino)phenyl)-2-phenylacetamide (1) (i.e., Compound NUCC-0226539)

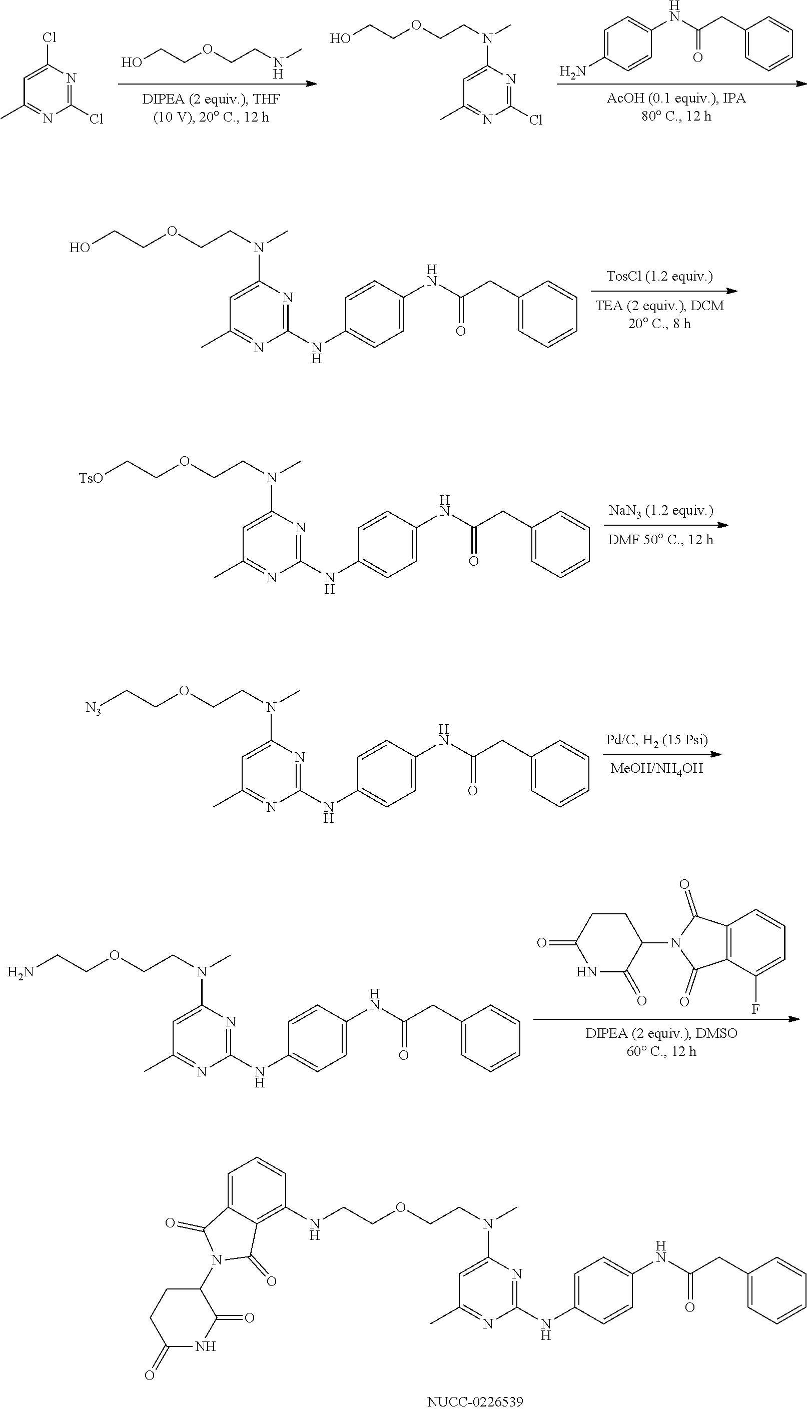

To a mixture of 2-(2-(methylamino)ethoxy)ethan-1-ol (1.10 g, 9.2 mmol) in tetrahydrofuran (10 mL) was added 2,4-dichloro-6-methylpyrimidine, 14 (1 g, 6.13 mmol) and DIPEA (1.59 g, 2.14 mL). The mixture was stirred at 20° C. for 12 hrs. LCMS showed all starting material was consumed and around 50% desired product could be detected. The reaction mixture was diluted with water (10 mL) and extracted with ethyl acetate (15 mL×2). The combined organic layers were washed with aqueous NaCl (20 mL×2), dried over Na₂SO₄, filtered and concentrated under reduced pressure to give a residue. The residue was purified by column chromatography (SiO₂, dichloromethane/methyl alcohol=100:0 to 2:1) to give 2-(2-((2-chloro-6-methylpyrimidin-4-yl)(methyl)amino)ethoxy)ethan-1-ol, 15 (0.5 g, yield 31%) as a light yellow solid. ¹H NMR: (400 MHz, CDCl₃): δ 1.86-2.01 (m, 1H) 2.35 (s, 3H) 3.11 (br s, 3H) 3.54-3.62 (m, 2H) 3.67-3.85 (m, 6H) 6.15-6.25 (m, 1H).

To a mixture of N-(4-aminophenyl)-2-phenylacetamide (920 mg, 4.07 mmol) in isopropan-2-ol (5 mL) was added 2-(2-((2-chloro-6-methylpyrimidin-4-yl)(methyl)amino)ethoxy)ethan-1-ol, 15 (0.5 g, 2.03 mmol) and acetic acid (8.64 mg, 143.79 μmol, 8.22 μL). The mixture was stirred at 80° C. for 12 hrs. LCMS showed all starting material was consumed and 60% of desired compound was detected. The reaction mixture was diluted with water (15 mL) and extracted with ethyl acetate (20 mL). The combined organic layers were washed with aqueous NaCl (20 mL), dried over Na₂SO₄, filtered and concentrated under reduced pressure to give a residue. The residue was purified by column chromatography (SiO₂, dichloromethane/methyl alcohol=100:0 to 3:1) to give N-(4-((4-((2-(2-hydroxyethoxy)ethyl)(methyl)amino)-6-methylpyrimidin-2-yl)amino)phenyl)-2-phenylacetamide, 18 (0.8 g, yield 81%) as a light yellow solid. ¹H NMR: (400 MHz, DMSO) δ 1.24-1.30 (m, 1H) 2.25-2.31 (m, 3H) 3.10-3.19 (m, 3H) 3.39-3.50 (m, 4H) 3.61-3.64 (m, 4H) 3.67-3.83 (m, 2H) 4.49-4.68 (m, 1H) 6.29-6.41 (m, 1H) 7.21-7.27 (m, 1H) 7.29-7.38 (m, 4H) 7.44-7.52 (m, 2H) 7.53-7.64 (m, 2H) 9.67-9.81 (m, 1H) 10.19-10.30 (m, 1H).

A mixture of N-(4-((4-((2-(2-hydroxyethoxy)ethyl)(methyl)amino)-6-methylpyrimidin-2-yl)amino)phenyl)-2-phenylacetamide, 18 (0.5 g, 1.1 mmol), TosCl (262 mg, 1.13 mol), and TEA (232 mg, 2.3 mmol) in DCM (5 mL) was degassed and purged with N₂ for 3 times at 0° C., and the mixture was stirred at 20° C. for 8 hrs. LCMS showed all starting material was consumed and 60% of desired compound was detected. The reaction mixture was diluted with water (20 mL) and extracted with DCM (10 mL). The organic layers were washed with aqueous NaCl (20 mL), dried over Na₂SO₄, filtered, and concentrated under reduced pressure to give a residue. The residue was purified by column chromatography (SiO₂, dichloromethane/methyl alcohol=100:0 to 3:1) to give 2-(2-(methyl(6-methyl-2-((4-(2-phenylacetamido)phenyl)amino)pyrimidin-4-yl)amino)ethoxy)ethyl 4-methylbenzenesulfonate, 21 (0.2 g, yield 28%) as a light yellow solid. ¹H NMR: (400 MHz, CDCl₃): δ 2.21-2.28 (m, 3H) 2.39-2.47 (m, 3H) 2.98-3.06 (m, 3H) 3.52-3.77 (m, 8H) 4.12 (s, 2H) 5.72-5.84 (m, 1H) 6.82-6.90 (m, 1H) 7.29-7.38 (m, 7H) 7.40-7.47 (m, 3H) 7.49-7.55 (m, 2H) 7.73-7.82 (m, 2H).

To a mixture of 2-(2-(methyl(6-methyl-2-((4-(2-phenylacetamido)phenyl)amino)pyrimidin-4-yl)amino)ethoxy)ethyl 4-methylbenzenesulfonate, 21 (0.2 g, 0.3 mmol) in N,N-dimethylformamide (2 mL) was added sodium azide (26 mg, 0.46 mmol). The mixture was stirred at 50° C. for 12 hrs. LCMS showed all starting material was consumed and 62% of desired product could be detected. The reaction solution was diluted with water (10 mL) and extracted with ethyl acetate (20 mL). The combined organic layers were washed with aqueous NaCl (5 mL), dried over Na₂SO₄, filtered and concentrated under reduced pressure to give a residue. The residue was purified by prep-TLC (SiO₂, dichloromethane/methyl alcohol=100:0 to 3:1) to give N-(4-((4-((2-(2-azidoethoxy)ethyl)(methyl)amino)-6-methylpyrimidin-2-yl)amino)phenyl)-2-phenylacetamide, 24 (0.15 g, yield 95%) as a yellow oil.

¹H NMR: (400 MHz, CDCl₃): δ 2.14-2.20 (m, 3H) 3.00-3.09 (m, 3H) 3.34-3.40 (m, 2H) 3.56-3.73 (m, 8H) 5.90-6.02 (m, 1H) 7.18-7.35 (m, 5H) 7.39-7.48 (m, 2H) 7.58-7.71 (m, 2H) 8.85-8.98 (m, 1H) 9.94-10.01 (m, 1H).

To a mixture of N-(4-((4-((2-(2-azidoethoxy)ethyl)(methyl)amino)-6-methylpyrimidin-2-yl)amino)phenyl)-2-phenylacetamide, 24 (0.15 g, 0.3 mmol) in methanol (3 mL) and ammonium hydroxide (1 mL) was added Pd/C (60.38 mg, 20%). The mixture was stirred at 20° C. for 12 hrs under H₂ (15 Psi). LCMS showed all starting material was consumed and 67% of desired compound was detected. The product was filtered and the filtrate was concentrated under high vacuum at 40° C. under high vacuum to give a white solid. The crude product N-(4-((4-((2-(2-aminoethoxy)ethyl)(methyl)amino)-6-methylpyrimidin-2-yl)amino)phenyl)-2-phenylacetamide, 27 (0.4 g, yield 56%) was used in the next step without further purification. ¹H NMR: (400 MHz, DMSO) δ 2.15-2.18 (m, 3H) 2.59-2.64 (m, 2H) 3.01 (br s, 3H) 3.15-3.19 (m, 2H) 3.35-3.36 (m, 1H) 3.55-3.62 (m, 4H) 3.63-3.73 (m, 2H) 4.05-4.13 (m, 1H) 5.87-6.02 (m, 1H) 7.19-7.27 (m, 1H) 7.29-7.34 (m, 4H) 7.39-7.46 (m, 2H) 7.61-7.67 (m, 2H) 8.81-8.99 (m, 1H) 9.90-10.01 (m, 1H).

To a solution of N-(4-((4-((2-(2-aminoethoxy)ethyl)(methyl)amino)-6-methylpyrimidin-2-yl)amino)phenyl)-2-phenylacetamide, 27 (50 mg, 115.07 μmol) and 2-(2,6-dioxopiperidin-3-yl)-4-fluoroisoindoline-1,3-dione (38.14 mg, 138.08 μmol) in DMSO (1 mL) was added DIPEA (29.74 mg, 230.13 μmol, 40.08 μL). The reaction mixture was stirred at 60° C. for 12 hr. LCMS showed all starting material was consumed and 25% of desired compound was detected. The reaction mixture was filtered and the filtrate was concentrated under high vacuum at 40° C. The residue was purified by prep-HPLC (Phenomenex luna C18 100×40 mm×5 μm, mobile phase: [water(0.1% TFA)-ACN]: 25%-53%, 8 min) to give N-(4-((4-((2-(2-((2-(2,6-dioxopiperidin-3-yl)-1,3-dioxoisoindolin-4-yl)amino)ethoxy)ethyl)(methyl)amino)-6-methylpyrimidin-2-yl)amino)phenyl)-2-phenylacetamide (1, 10 mg, yield 13%) as a yellow solid.¹H NMR (400 MHz, DMSO-d6): δ 1.99 (br s, 1H), 2.18-2.30 (m, 3H), 2.56-2.62 (m, 2H), 2.85 (br s, 1H), 2.94-3.04 (m, 1H), 3.09-3.19 (m, 3H), 3.52 (br s, 2H), 3.63 (s, 6H), 3.75 (br s, 1H), 5.03 (br s, 1H), 6.30-6.56 (m, 1H), 7.01 (d, J=7.03 Hz, 1H), 7.09 (br s, 1H), 7.21-7.28 (m, 1H), 7.29-7.36 (m, 4H), 7.43 (br s, 2H), 7.48-7.55 (m, 1H), 7.60 (br d, J=8.82 Hz, 2H), 9.75 (br s, 1H), 10.19 (s, 1H), 11.04-11.17 (m, 1H), 12.33 (br s, 1H). HRMS (ESI+): m/z calcd for C₃₇H₃₈N₈O₆: 691.2988 [M+H]⁺; found: 691.2903, [M+H]⁺.

### 2-((2-(2,6-dioxopiperidin-3-yl)-1,3-dioxoisoindolin-4-yl)oxy)-N-(2-(2-(methyl(6-methyl-2-((4-(2-phenylacetamido)phenyl)amino)pyrimidin-4-yl)amino)ethoxy)ethyl)acetamide, (4) (i.e., Compound NUCC-0226540)

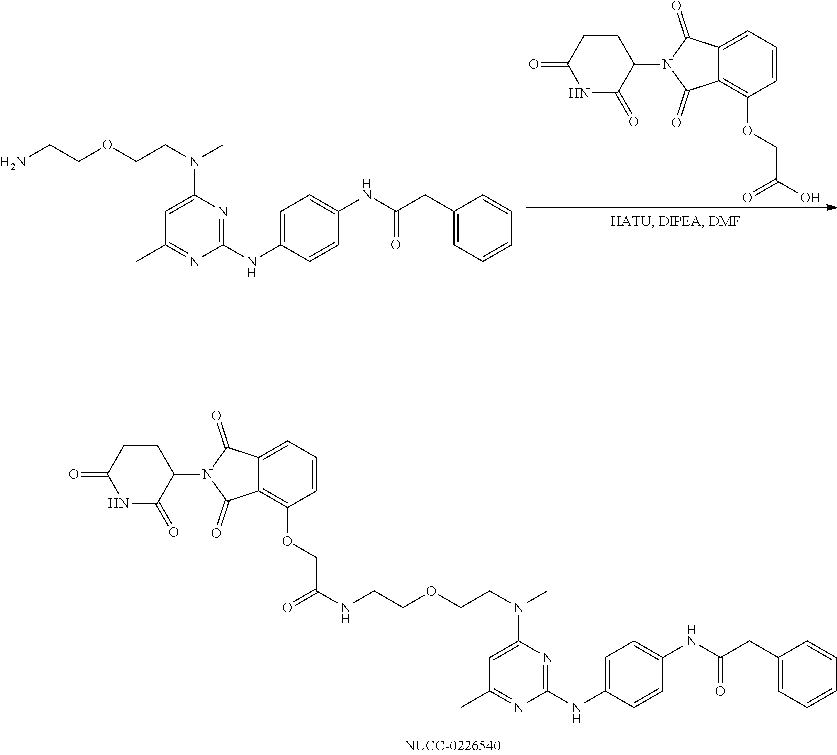 [SMILES: Cc1cc(N(C)CCOCCN)nc(Nc2ccc(NC(=O)Cc3ccccc3)cc2)n1 ; O=C(O)COc1cccc2c1C(=O)N(C1CCC(=O)NC1=O)C2=O ; Cc1cc(N(C)CCOCCNC(=O)COc2cccc3c2C(=O)N(C2CCC(=O)NC2=O)C3=O)nc(Nc2ccc(NC(=O)Cc3ccccc3)cc2)n1]

To a mixture of 2-((2-(2,6-dioxopiperidin-3-yl)-1,3-dioxoisoindolin-4-yl)oxy)acetic acid (40 mg, 0.11 mmol) and N-(4-((4-((2-(2-aminoethoxy)ethyl)(methyl)amino)-6-methylpyrimidin-2-yl)amino)phenyl)-2-phenylacetamide, 27 (36 mg, 0.11 mmol) in DMF (1 mL) was added DIPEA (23 mg, 191.34 μmol) and HATU (52 mg, 143.50 μmol). The mixture was stirred at 20° C. for 12 hrs. LC-MS showed all starting material was consumed and 25% of desired compound was detected. The reaction mixture was filtered and the filtrate was concentrated to give a residue which was purified by prep-HPLC (Phenomenex luna C18 100×40 mm×5 μm, mobile phase: [water(0.1% TFA)-ACN]: 25%-53%, 8 min) to give 2-((2-(2,6-dioxopiperidin-3-yl)-1,3-dioxoisoindolin-4-yl)oxy)-N-(2-(2-(methyl(6-methyl-2-((4-(2-phenylacetamido)phenyl)amino)pyrimidin-4-yl)amino)ethoxy)ethyl)acetamide, (4, 19 mg, yield 28%) as a white solid. ¹H NMR (400 MHz, DMSO-d6): δ 2.01 (br d, J=10.76 Hz, 1H), 2.25 (s, 3H), 2.58 (br s, 2H), 2.77-2.95 (m, 2H), 3.14 (br s, 3H), 3.30 (br s, 2H), 3.39-3.43 (m, 2H), 3.62 (s, 4H), 3.74 (br s, 1H), 4.74 (s, 2H), 5.10 (dd, J=12.90, 5.20 Hz, 1H), 6.21-6.56 (m, 1H), 7.20-7.28 (m, 1H), 7.32 (d, J=4.77 Hz, 3H), 7.36-7.45 (m, 3H), 7.49 (br d, J=6.97 Hz, 1H), 7.59 (br d, J=8.80 Hz, 2H), 7.75-7.83 (m, 1H), 7.89 (br s, 1H), 9.68 (br s, 1H), 10.18 (s, 1H), 11.12 (br s, 1H) HRMS (ESI+): m/z calcd for C₃₉H₄₁N₈O₈: 749.3042 [M+H]⁺; found: 749.3045, [M+H]⁺.

### N-(4-((4-((2-(2-(2-(2-((2-(2,6-dioxopiperidin-3-yl)-1,3-dioxoisoindolin-4-yl)amino)ethoxy)ethoxy)ethoxy)ethyl)(methyl)amino)-6-methylpyrimidin-2-yl)amino)phenyl)-2-phenylacetamide (2)

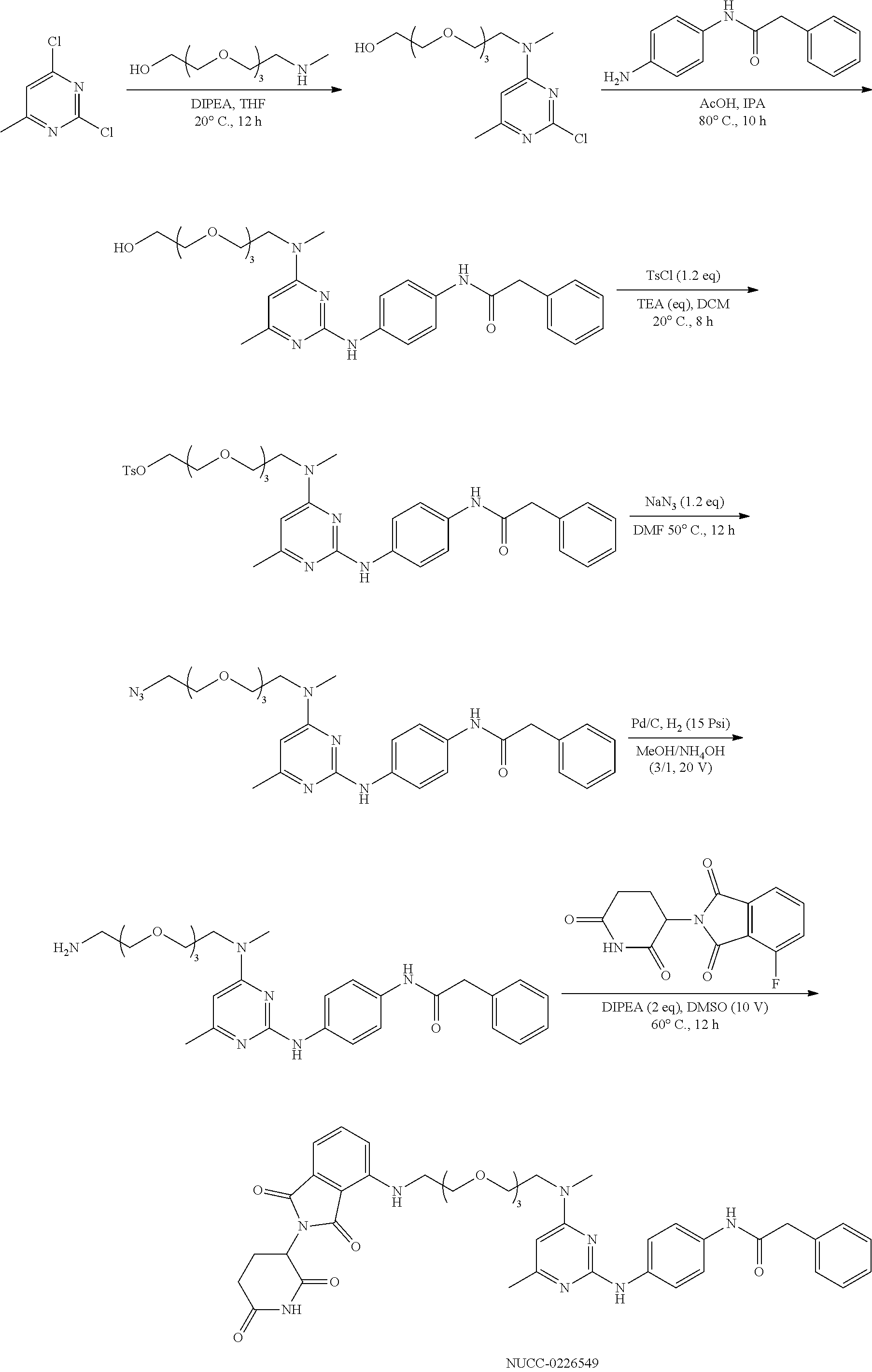

To a mixture of 2-((1-(2-methoxyethoxy)-3-(methylamino)propan-2-yl)oxy)ethan-1-ol (2.86 g, 13.80 mmol) in tetrahydrofuran (28 mL) was added 2,4-dichloro-6-methylpyrimidine, 14 (1.5 g, 9.20 mmol) and DIPEA (2.38 g, 18.40 mmol, 3.21 mL). The mixture was stirred at 20° C. for 12 hrs. LCMS showed all starting material was consumed and around 50% desired product could be detected. The reaction mixture was diluted with water (20 mL) and extracted with ethyl acetate (20 mL×2). The combined organic layers were washed with aqueous NaCl (20 mL×2), dried over Na₂SO₄, filtered and concentrated under reduced pressure to give a residue. The residue was purified by column chromatography (SiO₂, dichloromethane/methyl alcohol=100:0 to 10:1) to give 2-(2-chloro-6-methylpyrimidin-4-yl)-5,8,11-trioxa-2-azatridecan-13-ol, 16 (1.34 g, yield 39%) as a light yellow solid. ¹H NMR: (400 MHz, CDCl₃) δ 2.27 (s, 3H) 3.04 (br s, 3H) 3.52-3.57 (m, 9H) 3.57-3.62 (m, 5H) 3.63-3.67 (m, 3H) 6.14 (br s, 1H).

To a mixture of N-(4-aminophenyl)-2-phenylacetamide (1.30 g, 5.76 mmol) in isopropan-2-ol (13 mL) was added 2-(2-chloro-6-methylpyrimidin-4-yl)-5,8,11-trioxa-2-azatridecan-13-ol, 16 (0.48 g, 1.44 mmol) and acetic acid (8.64 mg, 143.79 μmol, 8.22 μL). The mixture was stirred at 80° C. for 12 hrs. LCMS showed all starting material was consumed and 60% of desired compound was detected. The reaction mixture was diluted with water (15 mL) and extracted with ethyl acetate (20 mL). The combined organic layers were washed with aqueous NaCl (20 mL), dried over Na₂SO₄, filtered, and concentrated under reduced pressure to give a residue. The residue was purified by column chromatography (SiO₂, dichloromethane/methyl alcohol=100:0 to 10:1) to give N-(4-((4-((2-(2-(2-(2-hydroxyethoxy)ethoxy)ethoxy)ethyl)(methyl)amino)-6-methylpyrimidin-2-yl)amino)phenyl)-2-phenylacetamide, 19 (1.5 g, yield 64%) as a light yellow solid. ¹H NMR: (400 MHz, DMSO) δ 2.31 (br d, J=8.07 Hz, 3H), 3.18 (br d, J=7.95 Hz, 3H), 3.38 (br d, J=5.01 Hz, 4H), 3.43-3.48 (m, 11H), 3.65 (s, 3H), 6.38-6.51 (m, 1H), 7.25 (br d, J=6.85 Hz, 1H), 7.29-7.37 (m, 4H), 7.40-7.49 (m, 2H), 7.63 (br d, J=8.07 Hz, 2H), 10.10-10.18 (m, 1H), 10.34 (br s, 1H), 12.66 (br s, 1H).

To a mixture of N-(4-((4-((2-(2-(2-(2-hydroxyethoxy)ethoxy)ethoxy)ethyl)(methyl)amino)-6-methylpyrimidin-2-yl)amino)phenyl)-2-phenylacetamide, 19 (1.5 g, 2.86 mmol) in dichloromethane (15 mL) was added tosyl chloride (655.38 mg, 3.44 mmol) and triethylamine (579.74 mg, 5.73 mmol, 797.45 μL). The mixture was stirred at 20° C. for 12 hrs. LCMS showed all starting material was consumed and 60% of desired compound was detected. The reaction mixture was diluted with water (20 mL) and extracted with ethyl acetate (20 mL). The organic layers were washed with aqueous NaCl (20 mL), dried over Na₂SO₄, filtered and concentrated under reduced pressure to give a residue. The residue was purified by column chromatography (SiO₂, dichloromethane/methyl alcohol=100:0 to 10:1) to give 2-(6-methyl-2-((4-(2-phenylacetamido)phenyl)amino)pyrimidin-4-yl)-5,8,11-trioxa-2-azatridecan-13-yl 4-methylbenzenesulfonate, 22 (1.5 g, yield 70%) as a light yellow solid. ¹H NMR: (400 MHz, CDCl₃): δ 2.24-2.29 (m, 1H), 2.27 (s, 2H), 2.43 (s, 3H), 3.10 (s, 3H), 3.57 (d, J=2.93 Hz, 8H), 3.65-3.69 (m, 4H), 3.73 (s, 4H), 5.84 (s, 1H), 7.30-7.43 (m, 11H), 7.52 (br d, J=8.68 Hz, 2H), 7.78 (d, J=8.19 Hz, 2H).

To a mixture of 2-(6-methyl-2-((4-(2-phenylacetamido)phenyl)amino)pyrimidin-4-yl)-5,8,11-trioxa-2-azatridecan-13-yl 4-methylbenzenesulfonate, 22 (1 g, 1.48 mmol) in N,N-dimethylformamide (10 mL) was added sodium azide (115.09 mg, 1.77 mmol). The mixture was stirred at 50° C. for 12 hrs. LCMS showed all starting material was consumed and 64% of desired product could be detected. The reaction solution was diluted with water (10 mL) and extracted with ethyl acetate (20 mL). The combined organic layers were washed with aqueous NaCl (5 mL), dried over Na₂SO₄, filtered and concentrated under reduced pressure to give a residue. The residue was purified by prep-TLC (SiO₂, dichloromethane/methyl alcohol=100:0 to 5:1) to give N-(4-((4-((2-(2-(2-(2-azidoethoxy)ethoxy)ethoxy)ethyl)(methyl)amino)-6-methylpyrimidin-2-yl)amino)phenyl)-2-phenylacetamide, 25 (0.6 g, yield 67%) as a dark brown solid. ¹H NMR: (400 MHz, CDCl₃) δ 2.28 (s, 3H), 3.11 (s, 3H), 3.35-3.39 (m, 2H), 3.58-3.62 (m, 1H), 3.61 (br d, J=4.40 Hz, 3H), 3.64 (s, 4H), 3.65-3.70 (m, 4H), 3.74 (s, 4H), 5.84 (s, 1H), 7.11 (br s, 1H), 7.32-7.44 (m, 8H), 7.52 (br d, J=8.80 Hz, 2H).

To a mixture of N-(4-((4-((2-(2-(2-(2-azidoethoxy)ethoxy)ethoxy)ethyl)(methyl)amino)-6-methylpyrimidin-2-yl)amino)phenyl)-2-phenylacetamide, 25 (0.6 g, 1.09 mmol) in methanol (9 mL) and ammonia hydrate (3 mL) was added Pd/C (116.38 mg, 20%). The mixture was stirred at 20° C. for 12 hrs under H₂ (15 Psi). LCMS showed all starting material was consumed and 70% of desired compound was detected. The product was filtered and the filtrate was concentrated under high vacuum at 40° C. under high vacuum to give a white solid. The crude product N-(4-((4-((2-(2-(2-(2-aminoethoxy)ethoxy)ethoxy)ethyl)(methyl)amino)-6-methylpyrimidin-2-yl)amino)phenyl)-2-phenylacetamide, 28 (0.4 g, yield 56%) was used in the next step without further purification.

¹H NMR: (400 MHz, CDCl₃) δ 2.20 (br s, 1H), 2.26 (s, 3H), 2.34-2.42 (m, 1H), 2.86 (t, J=5.14 Hz, 2H), 3.08 (s, 3H), 3.49-3.53 (m, 2H), 3.60 (br s, 8H), 3.64-3.69 (m, 2H), 3.74 (s, 4H), 5.82 (s, 1H), 7.30-7.45 (m, 8H), 7.47-7.55 (m, 3H).

To a mixture of N-(4-((4-((2-(2-(2-(2-aminoethoxy)ethoxy)ethoxy)ethyl)(methyl)amino)-6-methylpyrimidin-2-yl)amino)phenyl)-2-phenylacetamide, 28 (100 mg, 191.34 μmol) in DMSO (1 mL) was added 2-(2,6-dioxopiperidin-3-yl)-4-fluoroisoindoline-1,3-dione (63.42 mg, 229.60 μmol) and DIPEA (49.46 mg, 382.67 μmol, 66.65 μL). The mixture was stirred at 60° C. for 12 hrs. LCMS showed all starting material was consumed and 30% of desired compound was detected. The reaction mixture was filtered and the filtrate was concentrated under high vacuum at 40° C. The residue was purified by prep-HPLC (Phenomenex luna C18 100*40 mm*5 μm, mobile phase: [water(0.1% TFA)-ACN]: 25%-53%, 8 min) to give N-(4-((4-((2-(2-(2-(2-((2-(2,6-dioxopiperidin-3-yl)-1,3-dioxoisoindolin-4-yl)amino)ethoxy)ethoxy)ethoxy)ethyl)(methyl)amino)-6-methylpyrimidin-2-yl)amino)phenyl)-2-phenylacetamide (2, 8 mg, yield 5%) as a yellow solid. ¹H NMR: (400 MHz, DMSO) 2.01 (br s, 1H) (br s, 3H) 2.59 (br s, 2H) 2.80-2.92 (m, 1H) 3.11-3.18 (m, 3H) 3.48 (br d, J=12.23 Hz, 10H) 3.58 (br t, J=5.32 Hz, 4H) 3.61-3.77 (m, 5H) 5.04 (dd, J=12.90, 5.69 Hz, 1H) 6.33-6.51 (m, 1H) 7.03 (d, J=6.97 Hz, 1H) 7.10 (d, J=8.56 Hz, 1H) 7.25 (br d, J=3.18 Hz, 1H) 7.29-7.36 (m, 4H) 7.43 (br s, 2H) 7.52-7.64 (m, 3H) 9.67-9.90 (m, 1H) 10.20 (s, 1H) 11.09 (s, 1H) 12.26 (s, 1H).HRMS (ESI+): m/z calcd for C₄₁H₄₇N₈O₈: 779.3512 [M+H]⁺; found: 779.3533 [M+H]⁺.

### 2-((2-(2,6-dioxopiperidin-3-yl)-1,3-dioxoisoindolin-4-yl)oxy)-N-(2-(6-methyl-2-((4-(2-phenylacetamido)phenyl)amino)pyrimidin-4-yl)-5,8,11-trioxa-2-azatridecan-13-yl)acetamide (5) (i.e., compound NUCC-0226550)

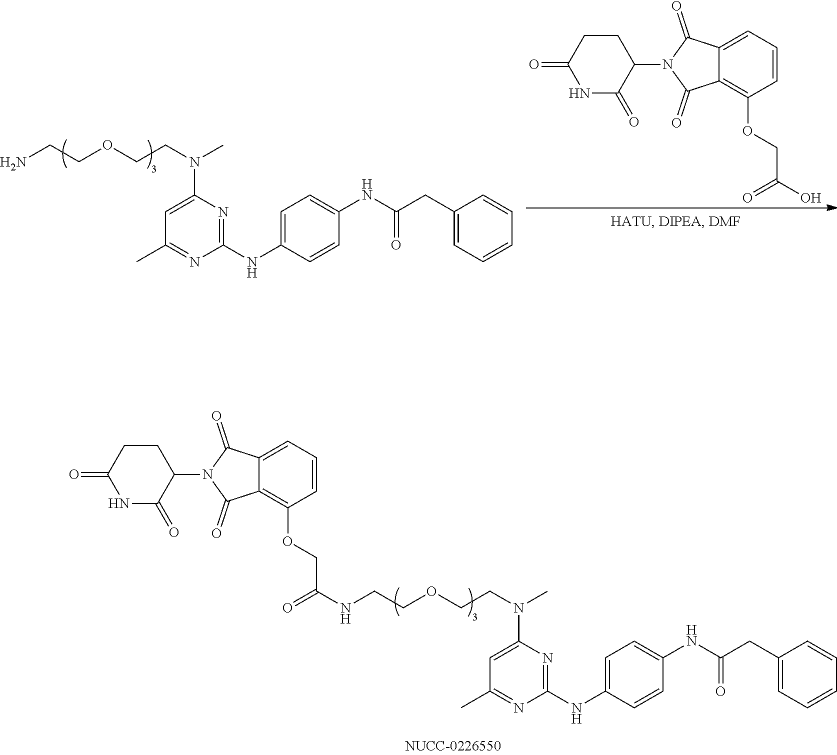

To the mixture of 2-((2-(2,6-dioxopiperidin-3-yl)-1,3-dioxoisoindolin-4-yl)oxy)acetic acid (38.14 μmol, 114.80 μmol, 1.2 eq) and N-(4-((4-((2-(2-(2-(2-aminoethoxy)ethoxy)ethoxy)ethyl)(methyl)amino)-6-methylpyrimidin-2-yl)amino)phenyl)-2-phenylacetamide, 28 (50 mg, 95.67 μmol, 1 eq) in DMF (1 mL) was added DIPEA (24.73 mg, 191.34 μmol, 2 eq) and HATU (54.56 mg, 143.50 μmol, 1.5 eq). The mixture was stirred at 20° C. for 12 hr. LC-MS showed all starting material was consumed and 30% of desired compound was detected. The reaction mixture was filtered and the filtrate was concentrated to give a residue which was purified by prep-HPLC (Phenomenex luna C18 100*40 mm*5 μm, mobile phase: [water(0.1% TFA)-ACN]: 25%-53%, 8 min) to give 2-((2-(2,6-dioxopiperidin-3-yl)-1,3-dioxoisoindolin-4-yl)oxy)-N-(2-(6-methyl-2-((4-(2-phenylacetamido)phenyl)amino)pyrimidin-4-yl)-5,8,11-trioxa-2-azatridecan-13-yl)acetamide, 5 (20 mg, yield 24%) as a white solid. ¹H NMR (400 MHz, DMSO-d6): 2.04 (br s, 1H), 2.30 (br s, 3H), 2.56 (br s, 1H), 2.59-2.62 (m, 1H), 2.59-2.62 (m, 1H), 2.60 (br s, 1H), 2.83-2.94 (m, 1H), 3.15 (s, 3H), 3.29 (br d, J=5.14 Hz, 1 H), 3.46-3.48 (m, 9H), 3.58 (br s, 2H), 3.63 (s, 3H), 3.72 (br d, J=16.26 Hz, 2H), 4.77 (s, 2H), 5.07-5.19 (m, 1H), 6.38-6.51 (m, 1H), 7.25 (br s, 1H), 7.33 (br d, J=3.42 Hz, 4H), 7.38 (br d, J=8.31 Hz, 2H), 7.42-7.46 (m, 1H), 7.49 (br d, J=7.34 Hz, 1H), 7.61 (br d, J=7.70 Hz, 2H), 7.77-7.82 (m, 1H), 7.97 (br s, 1H), 9.69 (br s, 1H), 10.20 (s, 1H), 11.11 (s, 1H), 12.14 (br s, 1 H). HRMS (ESI+): m/z calcd for C₄₃H₄₉N₈O₁₀: 837.3567 [M+H]⁺; found: 837.3579, [M+H]⁺.

### N-(4-((4-((17-((2-(2,6-dioxopiperidin-3-yl)-1,3-dioxoisoindolin-4-yl)amino)-3,6,9,12,15-pentaoxaheptadecyl)(methyl)amino)-6-methylpyrimidin-2-yl)amino)phenyl)-2-phenylacetamide (3) (i.e., compound NUCC-0226551)

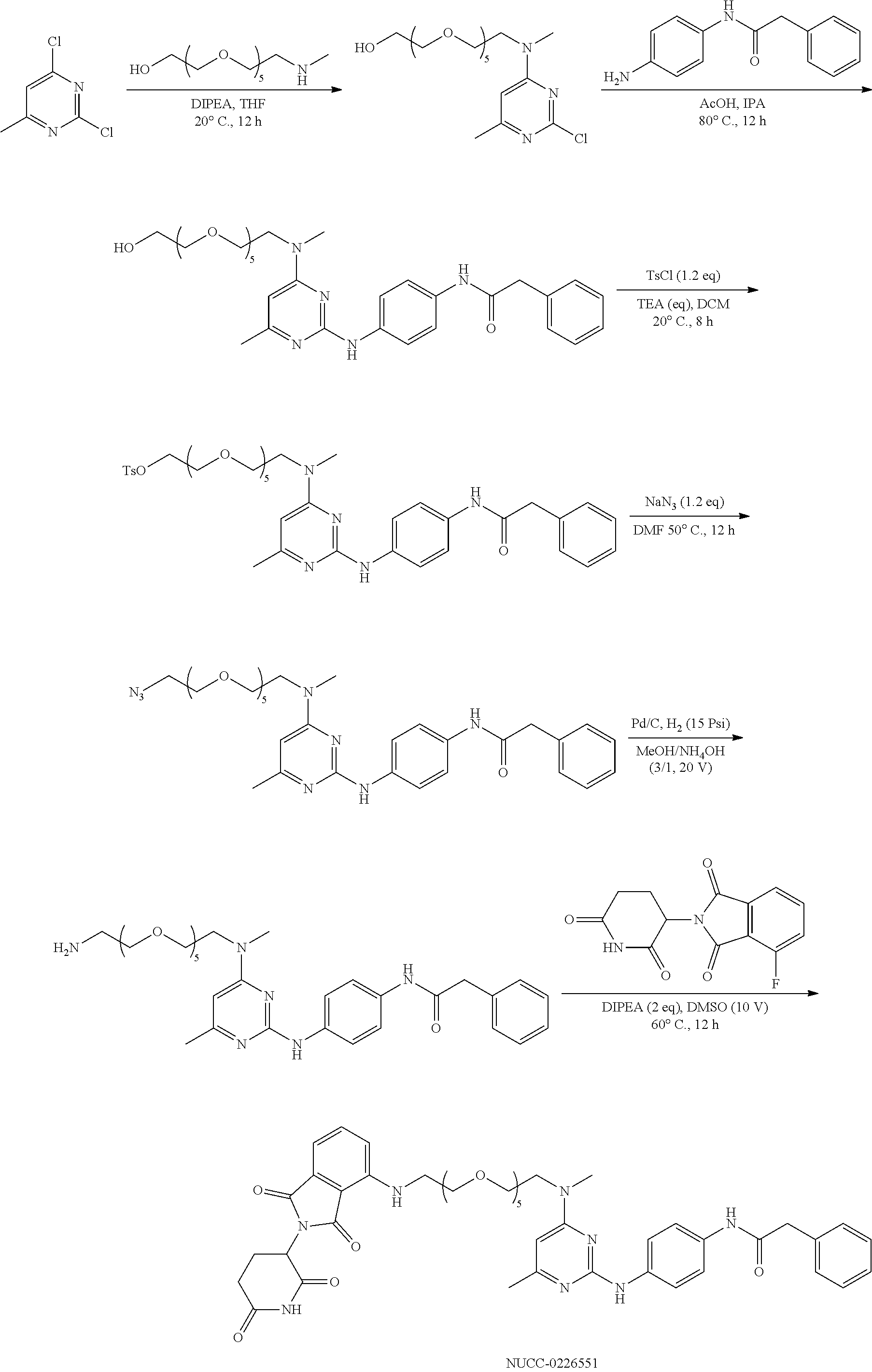

To a mixture of 13-((methylamino)methyl)-2,5,8,11,14-pentaoxahexadecan-16-ol (2.9 g, 9.82 mmol) in tetrahydrofuran (29 mL) was added 2,4-dichloro-6-methylpyrimidine, 14 (1.5 g, 9.82 mmol) and DIPEA (2.38 g, 19.64 mmol, 3.42 mL). The mixture was stirred at 20° C. for 12 hrs. LCMS showed all starting material was consumed and around 50% desired product could be detected. The reaction mixture was diluted with water (20 mL) and extracted with ethyl acetate (20 mL×2). The combined organic layers were washed with aqueous NaCl (20 mL×2), dried over Na₂SO₄, filtered and concentrated under reduced pressure to give a residue. The residue was purified by column chromatography (SiO₂, dichloromethane/methyl alcohol=100:0 to 10:1) to give 2-(2-chloro-6-methylpyrimidin-4-yl)-5,8,11,14,17-pentaoxa-2-azanonadecan-19-ol, 17 (0.89 g, yield 20.41%) as a light-yellow solid. ¹H NMR: (400 MHz, DMSO-d₆) δ ppm 2.23 (s, 3H) 3.04 (br s,3 H) 3.37-3.43 (m, 3H) 3.47-3.51 (m, 17H) 3.57-3.67 (m, 4H) 4.56 (t, J=5.32 Hz, 1 H) 6.50-6.59 (m, 1H).

To a mixture of N-(4-aminophenyl)-2-phenylacetamide (0.477 g, 2.11 mmol) in isopropan-2-ol (10 mL) and was added 2-(2-chloro-6-methylpyrimidin-4-yl)-5,8,11,14,17-pentaoxa-2-azanonadecan-19-ol, 17 (0.89 g, 2.11 mmol,) and acetic acid (12.67 mg, 0.211 mmol). The mixture was stirred at 80° C. for 12 hrs. LCMS showed all starting material was consumed and 60% of desired compound was detected. The reaction mixture was diluted with water (15 mL) and extracted with ethyl acetate (20 mL) three times. The combined organic layers were washed with aqueous NaCl (20 mL), dried over Na₂SO₄, filtered and concentrated under reduced pressure to give a residue. The residue was purified by column chromatography (SiO₂, dichloromethane/methyl alcohol=100:0 to 10:1) to give N-(4-((4-((17-hydroxy-3,6,9,12,15-pentaoxaheptadecyl)(methyl)amino)-6-methylpyrimidin-2-yl)amino)phenyl)-2-phenylacetamide, 20 (1 g, yield 54.25%) as a light yellow solid. ¹H NMR: (400 MHz, DMSO-d₆) δ ppm 2.31 (br s, 3H) 3.17 (br s, 4H) 3.45-3.50 (m, 20H) 3.59-3.65 (m, 4H) 6.34-6.54 (m, 1H) 7.21-7.35 (m, 5H) 7.43-7.51 (m, 2H) 7.63 (br d, J=8.55 Hz, 2H) 10.11 (br s, 1H) 10.34 (s, 1H).

To a mixture of N-(4-((4-((17-hydroxy-3,6,9,12,15-pentaoxaheptadecyl)(methyl)amino)-6-methylpyrimidin-2-yl)amino)phenyl)-2-phenylacetamide, 20 (1 μg, 2.86 μmmol) in dichloromethane (10 mL) was added tosyl chloride (655.38 mg, 3.44 mmol) and triethylamine (579.74 mg, 5.73 mmol). The mixture was stirred at 20° C. for 12 hrs. LCMS showed all starting material was consumed and 60% of desired compound was detected. The reaction mixture was diluted with water (20 mL) and extracted with ethyl acetate (20 mL). The organic layers were washed with aqueous NaCl (20 mL), dried over Na₂SO₄, filtered and concentrated under reduced pressure to give a residue. The residue was purified by column chromatography (SiO₂, dichloromethane/methyl alcohol=100:0 to 10:1) to give 2-(6-methyl-2-((4-(2-phenylacetamido)phenyl)amino)pyrimidin-4-yl)-5,8,11,14,17-pentaoxa-2-azanonadecan-19-yl 4-methylbenzenesulfonate, 23 (1.5 g, yield 70%) as a light yellow solid. ¹H NMR: (400 MHz, CHLOROFORM-d) δ ppm 2.29 (s, 3H) 2.44 (s, 3H) 3.12 (s, 3H) 3.50 (s, 2H) 3.56 (s, 7H) 3.62 (br s, 8H) 3.66 (br d, J=5.14 Hz, 4H) 3.73 (s, 4H) 4.12-4.16 (m, 2H) 5.85 (s, 1H) 7.33-7.42 (m, 10H) 7.52 (brd,J=8.56 Hz, 2H) 7.79 (d, J=8.19 Hz, 2H).

To a mixture of 2-(6-methyl-2-((4-(2-phenylacetamido)phenyl)amino)pyrimidin-4-yl)-5,8,11,14,17-pentaoxa-2-azanonadecan-19-yl 4-methylbenzenesulfonate, 23 (1 g, 1.31 mmol) in N, N-dimethylformamide (10 mL) was added sodium azide (101.85 mg, 1.57 mmol). The mixture was stirred at 50° C. for 12 hrs. LCMS showed all starting material was consumed and 64% of desired product could be detected. The reaction solution was diluted with water (10 mL) and extracted with ethyl acetate (20 mL)three times. The combined organic layers were washed with aqueous NaCl (5 mL), dried over Na₂SO₄, filtered and concentrated under reduced pressure to give a residue. The residue was purified by prep-TLC (SiO₂, dichloromethane/methyl alcohol=100:0 to 5:1) to give N-(4-((4-((17-azido-3,6,9,12,15-pentaoxaheptadecyl)(methyl)amino)-6-methylpyrimidin-2-yl)amino)phenyl)-2-phenylacetamide, 26 (1 g, yield 90.21%) as a dark brown solid. ¹H NMR: (400 MHz, CHLOROFORM-d) δ ppm 2.29 (s, 3H) 3.11 (br s, 3H) 3.35-3.40 (m, 2H) 3.58 (br s, 4H) 3.65 (br d, J=5.99 Hz, 16H) 3.74 (br s, 4H) 5.84 (s, 1H) 7.30-7.55 (m, 11H).

To a mixture of N-(4-((4-((17-azido-3,6,9,12,15-pentaoxaheptadecyl)(methyl)amino)-6-methylpyrimidin-2-yl)amino)phenyl)-2-phenylacetamide, 26 (1 g, 1.18 mmol) in methanol (15 mL) and ammonia hydrate (5 mL) was added Pd/C (100 mg, 10%). The mixture was stirred at 20° C. for 12 hrs under H₂ (15 Psi). LCMS showed all starting material was consumed and 70% of desired compound was detected. The product was filtered and the filtrate was concentrated under high vacuum at 40° C. under high vacuum to give a white solid. The crude product N-(4-((4-((17-amino-3,6,9,12,15-pentaoxaheptadecyl)(methyl)amino)-6-methylpyrimidin-2-yl)amino)phenyl)-2-phenylacetamide, 29 (360 mg, yield 50.1%) was used in the next step without further purification.¹H NMR: (400 MHz, CHLOROFORM-d) δ ppm 2.26 (s, 3H) 3.07 (s, 3H) 3.49-3.68 (m, 24H) 3.72-3.78 (m, 4H) 5.81 (s, 1H) 7.29-7.41 (m, 6H) 7.42-7.56 (m, 4H).

To a mixture of N-(4-((4-((17-amino-3,6,9,12,15-pentaoxaheptadecyl)(methyl)amino)-6-methylpyrimidin-2-yl)amino)phenyl)-2-phenylacetamide, 29 (50 mg, 69.59 μmol) in DMSO (0.5 mL) was added 2-(2,6-dioxopiperidin-3-yl)-4-fluoroisoindoline-1,3-dione (23.07 mg, 83.50 μmol) and DIPEA (17.99 mg, 139.17 μmol). The mixture was stirred at 60° C. for 12 hrs. LCMS showed all starting material was consumed and 30% of desired compound was detected. The reaction mixture was filtered and the filtrate was concentrated under high vacuum at 40° C. The residue was purified by prep-HPLC (Phenomenex luna C18 100*40 mm*5 μm, mobile phase: [water(0.1% TFA)-ACN]: 25%-53%, 8 min) to give N-(4-((4-((17-((2-(2,6-dioxopiperidin-3-yl)-1,3-dioxoisoindolin-4-yl)amino)-3,6,9,12,15-pentaoxaheptadecyl)(methyl)amino)-6-methylpyrimidin-2-yl)amino)phenyl)-2-phenylacetamide (3, 8 mg, yield 12.86%) as a yellow solid. ¹H NMR: (400 MHz, DMSO-d6) δ ppm 2.01 (br d, J=10.76 Hz, 1H), 2.29 (br s, 3H), 2.55-2.57 (m, 2H), 2.81-2.90 (m, 1H), 3.15 (s, 3H), 3.44 (br s, 9H), 3.52 (br d, J=7.09 Hz, 11H), 3.57-3.64 (m, 10H), 3.69 (br s, 5H), 5.05 (dd, J=13.02, 5.20 Hz, 1H), 6.36-6.52 (m, 1H), 6.55-6.61 (m, 1H), 7.03 (d, J=6.97 Hz, 1H), 7.12 (d, J=8.56 Hz, 1H), 7.24 (br s, 1H), 7.29-7.35 (m, 4H), 7.43 (br s, 2H), 7.53-7.65 (m, 3H), 9.73 (s, 1H), 10.20 (s, 1H), 11.09 (s, 1H), 12.14-12.81 (m, 1H). HRMS (ESI+): m/z calcd for C₄₅H₅₅N₈O₁₀: 867.4036 [M+H]⁺; found: 867.4054, [M+H]⁺. 2-((2-(2,6-dioxopiperidin-3-yl)-1,3-dioxoisoindolin-4-yl)oxy)-N-(2-(6-methyl-2-((4-(2-phenylacetamido)phenyl)amino)pyrimidin-4-yl)-5,8,11,14,17-pentaoxa-2-azanonadecan-19-yl)acetamide, (6) (i.e., Compound NUCC-0226552).

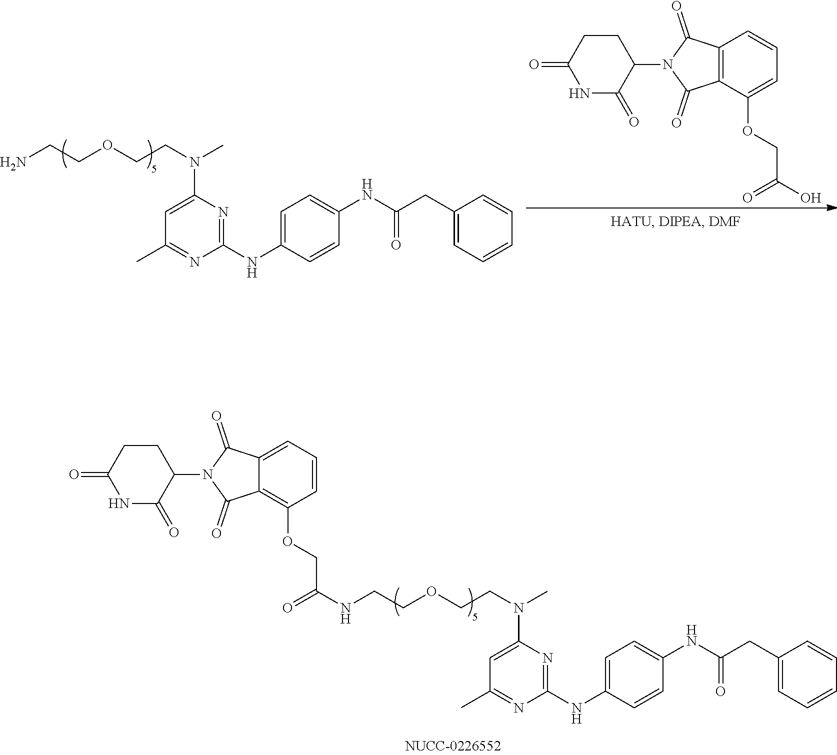

To the mixture of 2-((2-(2,6-dioxopiperidin-3-yl)-1,3-dioxoisoindolin-4-yl)oxy)acetic acid (27.75 mg, 83.50 μmol) and N-(4-((4-((17-amino-3,6,9,12,15-pentaoxaheptadecyl)(methyl)amino)-6-methylpyrimidin-2-yl)amino)phenyl)-2-phenylacetamide, 29 (50 mg, 69.59 μmol) in dimethylformamide (0.5 mL) was added DIPEA (17.99 mg, 139.17 μmol) and HATU (39.69 mg, 104.38 μmol). The mixture was stirred at 20° C. for 12 hr. LC-MS showed all starting material was consumed and 30% of desired compound was detected. The reaction mixture was filtered and the filtrate was concentrated to give residue which was purified by prep-HPLC (Phenomenex luna C18 100*40 mm*5 μm, mobile phase: [water(0.1% TFA)-ACN]: 25%-53%, 8 min) to give 2-((2-(2,6-dioxopiperidin-3-yl)-1,3-dioxoisoindolin-4-yl)oxy)-N-(2-(6-methyl-2-((4-(2-phenylacetamido)phenyl)amino)pyrimidin-4-yl)-5,8,11,14,17-pentaoxa-2-azanonadecan-19-yl)acetamide, (6, 30 mg, yield 44.74%) as a white solid. ¹H NMR: (400 MHz, DMSO-d6) δ ppm 1.98-2.06 (m, 1H), 2.29 (br s, 3H), 2.51-2.62 (m, 2H), 2.81-2.94 (m, 1H), 3.14 (s, 3H), 3.26-3.32 (m, 2H), 3.41-3.48 (m, 17H), 3.61 (s, 6H), 4.76 (s, 2H), 5.10 (dd, J=13.01, 5.29 Hz, 1H), 6.37-6.50 (m, 1H), 7.20-7.26 (m, 1H), 7.28-7.34 (m, 4H), 7.35-7.50 (m, 4H), 7.60 (br d, J=8.60 Hz, 2H), 7.79 (t, J=7.83 Hz, 1H), 7.99 (br t, J=5.51 Hz, 1H), 9.89 (br s, 1H), 10.21 (s, 1H), 11.11 (s, 1H), 12.44 (s, 1H). HRMS (ESI+): m/z calcd for C₄₇H₅₇N₈O₁₂: 925.4091 [M+H]⁺; found: 925.4097, [M+H]⁺.

### (2S,4R)-1-((S)-3,3-dimethyl-2-(3-(2-(methyl(6-methyl-2-((4-(2-phenylacetamido)phenyl)amino)pyrimidin-4-yl)amino)ethoxy)propanamido)butanoyl)-4-hydroxy-N-(4-(4-methylthiazol-5-yl)benzyl)pyrrolidine-2-carboxamide (7) (i.e., Compound NUCC-0226541)

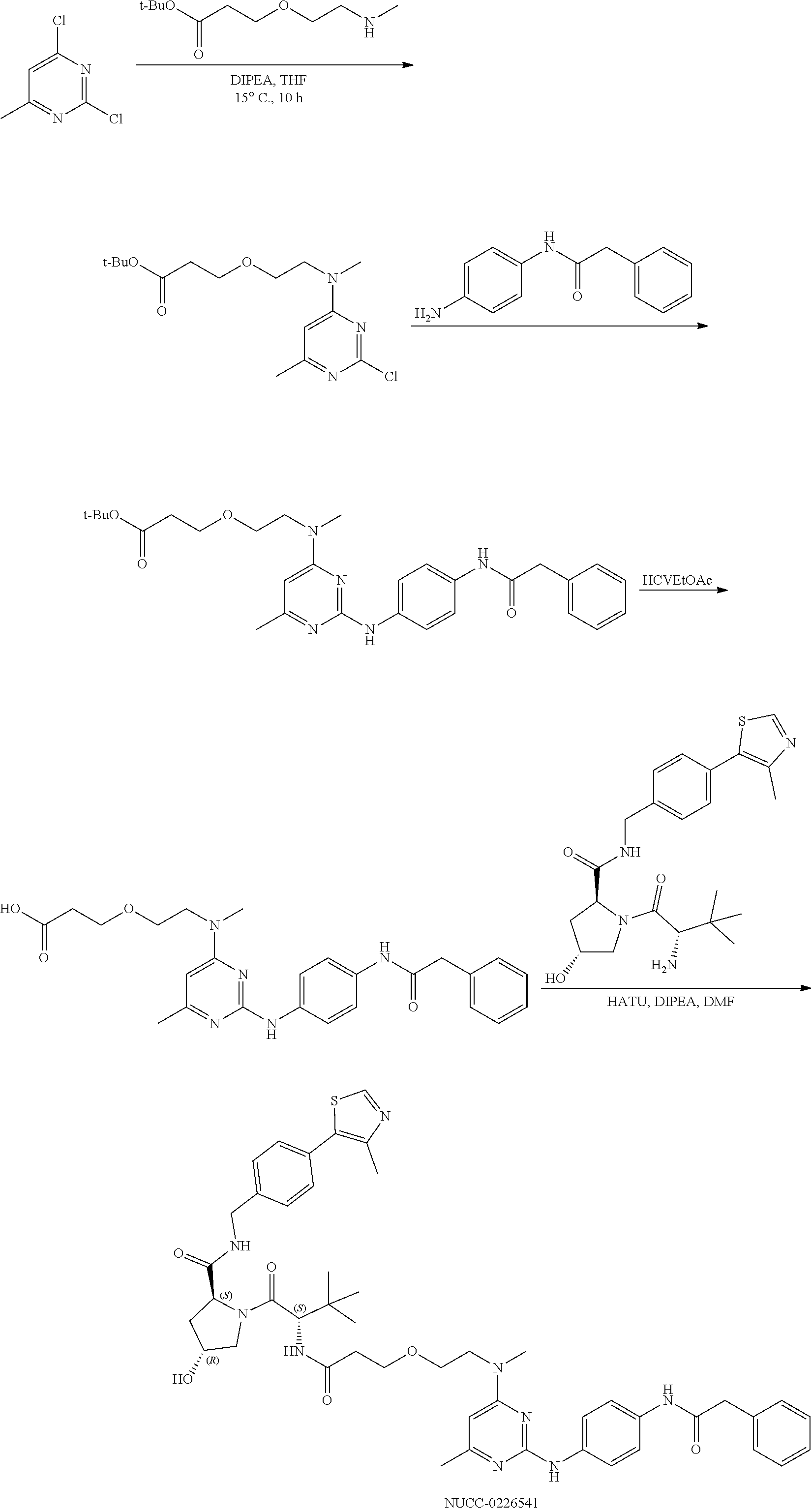

To a solution of tert-butyl 3-(2-(methylamino)ethoxy)propanoate (3.45 g, 9.20 mmol, 1.5 eq) and DIPEA (2.38 g, 18.39 mmol, 3.21 mL, 3 eq) in THF (50 mL) was added 2,4-dichloro-6-methyl-pyrimidine, 14 (1 g, 6.13 mmol, 1 eq) at 0° C. under N₂. After addition, the reaction was stirred at 15° C. for 10 hours. TLC (petroleum ether: ethyl acetate=3:1) showed the starting material was consumed and the desired product was obtained. The reaction was concentrated and purified by column chromatography on silica gel (pet. ether: ethyl acetate=100:1 to 100:20) to give tert-butyl 3-(2-((2-chloro-6-methylpyrimidin-4-yl)(methyl)amino)ethoxy)propanoate (30, 1.2 g, 3.64 mmol, yield 59.4%) as colorless oil. ¹H NMR: (400 MHz CDCl₃) δ=1.43 (s, 9H), 2.33 (s, 3H), 2.50-2.39 (m, 2H), 3.08 (br s, 3H), 3.74-3.52 (m, 6H), 6.17 (br s, 1H). The regioselectivity of this reaction was confirmed by NOE.

To a solution of N-(4-aminophenyl)-2-phenyl-acetamide (792.39 mg, 3.50 mmol, 1.05 eq) and AcOH (200.28 mg, 3.34 mmol, 190.74 μL, 1 eq) in i-PrOH (50 mL) was added tert-butyl 3-(2-((2-chloro-6-methylpyrimidin-4-yl)(methyl)amino)ethoxy)propanoate, 30 (1.1 g, 3.34 mmol, 1 eq) at 0° C. under N₂. After addition, the reaction was stirred at 80° C. for 10 hours. LCMS showed the starting material was consumed and the desired product was obtained. The reaction was cooled to 25° C. and filtered to obtain tert-butyl 3-(2-(methyl(6-methyl-2-((4-(2-phenylacetamido)phenyl)amino)pyrimidin-4-yl)amino)ethoxy)propanoate (35, 1.2 g, 2.31 mmol, yield 69.2%) as a brown solid. LCMS (ESI+): m/z 520.1 (M+H)⁺. ¹H-NMR (400 MHz, CHLOROFORM-d): 6=1.43-1.34 (m, 9H), 2.46-2.29 (m, 5H), 3.20-3.08 (m, 3H), 3.68-3.52 (m, 5H), 3.82-3.72 (m, 3H), 5.81 (s, 1H), 5.93 (s, 1H), 7.44-7.20 (m, 1H), 7.34-7.21 (m, 4H), 7.42-7.36 (m, 1H), 7.55 (d, J=8.8 Hz, 2H), 8.79-8.55 (m, 1H), 10.15-9.91 (m, 1H), 13.52-13.23 (m, 1H).

To a solution of tert-butyl 3-(2-(methyl(6-methyl-2-((4-(2-phenylacetamido)phenyl)amino)pyrimidin-4-yl)amino)ethoxy)propanoate, 35 (600 mg, 987.27 μmol, 1 eq) in ethyl acetate (20 mL) was added HCl/EtOAc (4 M, 6 mL) and the reaction was stirred at 25° C. for 12 hours. The reaction was concentrated to give 3-(2-(methyl(6-methyl-2-((4-(2-phenylacetamido)phenyl)amino)pyrimidin-4-yl)amino)ethoxy)propanoic acid (40, 400 mg, 73.5% yield) as a brown solid which was used in the next step without further purification.

To a solution of 3-(2-(methyl(6-methyl-2-((4-(2-phenylacetamido)phenyl)amino)pyrimidin-4-yl)amino)ethoxy)propanoic acid, 40 (60 mg, 129.44 μmol, 1 eq), (2S,4R)-1-((S)-2-amino-3,3-dimethylbutanoyl)-4-hydroxy-N-(4-(4-methylthiazol-5-yl)benzyl)pyrrolidine-2-carboxamide ((S,R,S)-AHPC, CAS 1448297-52-6 from Bide Pharmatech Ltd.) (55.73 mg, 129.44 μmol, 1 eq) and DIPEA (66.92 mg, 517.77 μmol, 90.19 μL, 4 eq) in DMF (4 mL) was added HATU (98.44 mg, 258.88 μmol, 2 eq) at 0° C. After addition, the reaction was stirred at 25° C. for 12 hours. LCMS showed the starting material was consumed and the desired product was detected. The reaction mixture was concentrated and purified by prep-HPLC (method: column-Phenomenex Gemini-NX C18 75*30 mm*3 μm; mobile phase: [water (0.05% NH₃H₂O+10 mM NH₄HCO₃)-MeCN]; 35%-55%, 8 min) to give (2S,4R)-1-((S)-3,3-dimethyl-2-(3-(2-(methyl(6-methyl-2-((4-(2-phenylacetamido)phenyl)amino)pyrimidin-4-yl)amino)ethoxy)propanamido)butanoyl)-4-hydroxy-N-(4-(4-methylthiazol-5-yl)benzyl)pyrrolidine-2-carboxamide (7, 35 mg, 39.95 μmol, 30.9% yield, 100% purity) as a white solid. ¹H NMR: (400 MHz, DMSO-d₆) δ 0.93 (s, 9H), 1.90 (ddd, J=4.5, 8.6, 12.8 Hz, 1H), 2.08-1.99 (m, 1H), 2.16 (s, 3H), 2.41-2.34 (m, 1H), 2.45-2.42 (m, 3H), 2.63-2.54 (m, 1H), 3.08-2.94 (m, 3H), 3.32-3.29 (m, 1H), 3.71-3.51 (m, 10H), 4.21 (dd, J=5.4, 15.9 Hz, 1H), 4.35 (br s, 1H), 4.47-4.39 (m, 2H), 4.56 (d, J=9.2 Hz, 1H), 5.14 (d, J=3.1 Hz, 1H), 5.94 (s, 1H), 7.26-7.18 (m, 1H), 7.45-7.28 (m, 10H), 7.63 (d, J=8.8 Hz, 2H), 7.95 (d, J=9.4 Hz, 1H), 8.57 (t, J=6.0 Hz, 1H), 8.90 (s, 1H), 8.97 (s, 1H), 9.97 (s, 1H). ¹³C NMR (126 MHz, DMSO) δ 16.39, 24.21, 26.76, 35.84, 36.07, 38.41, 42.06, 43.69, 48.94, 56.71, 56.88, 59.15, 67.40, 67.46, 68.40, 69.32, 93.20, 118.87, 119.88, 126.89, 127.83, 128.71, 129.07, 129.50, 130.05, 131.60, 132.63, 136.68, 137.57, 139.94, 148.14, 151.92, 159.53, 162.79, 165.44, 168.90, 169.96, 170.38, 172.39. HRMS (ESI+): m/z calcd for C₄₇H₅₈N₉O₆S: 876.4226 [M+H]⁺; found: 876.4227, [M+H]⁺.

### (2S,4S)-1-((S)-3,3-dimethyl-2-(3-(2-(methyl(6-methyl-2-((4-(2-phenylacetamido)phenyl)amino)pyrimidin-4-yl)amino)ethoxy)propanamido)butanoyl)-4-hydroxy-N-(4-(4-methylthiazol-5-yl)benzyl)pyrrolidine-2-carboxamide (12)

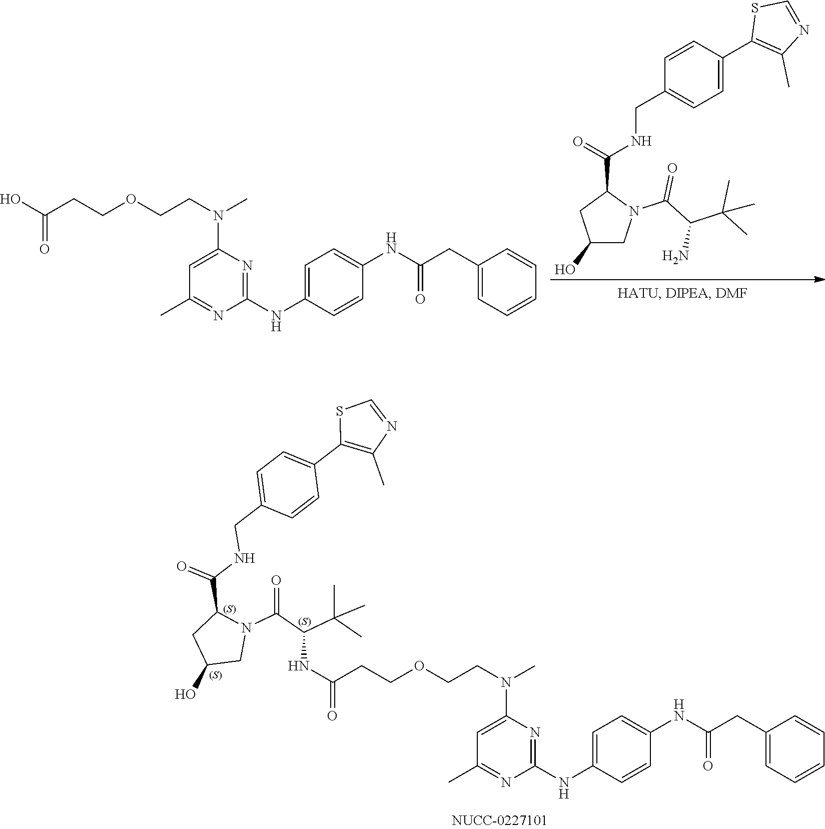

To a mixture of 3-(2-(methyl(6-methyl-2-((4-(2-phenylacetamido)phenyl)amino)pyrimidin-4-yl)amino)ethoxy)propanoic acid (40, 30.0 mg, 1 Eq, 64.7 μmol) in DMF (1 mL) was added DIPEA (25.1 mg, 33.8 μL, 3 Eq, 194 μmol) after which HATU (36.9 mg, 1.5 Eq, 97.1 μmol) was added. The mixture was stirred for 10 min after which (2S,4S)-1-((S)-2-amino-3,3-dimethylbutanoyl)-4-hydroxy-N-(4-(4-methylthiazol-5-yl)benzyl)pyrrolidine-2-carboxamide hydrochloride (30.2 mg, 1 Eq, 64.7 μmol, VHL negative control (S,S,S)-AHPC hydrochloride from MedChemExpress; Cat #HY-125845A) was added. After 2 h LCMS indicated product. The mixture was stirred for another 1 h after which it was purified by RP HPLC (Phenomenex Gemini-NX C18, 110 A, 150×21.2 mm×5 μm. Eluting with a gradient of 15% to 90% acetonitrile in water with 0.1% formic acid, a flow rate of 20 mL/min, gradient time of 30 min, and detection wavelength of 200 nm) to give (2S,4S)-1-((S)-3,3-dimethyl-2-(3-(2-(methyl(6-methyl-2-((4-(2-phenylacetamido)phenyl)amino)pyrimidin-4-yl)amino)ethoxy)propanamido)butanoyl)-4-hydroxy-N-(4-(4-methylthiazol-5-yl)benzyl)pyrrolidine-2-carboxamide (12, 25 mg, 41%). ¹H NMR (500 MHz, CD₃OD) δ 1.00 (s, 9H), 1.98 (dt, J=13.2, 4.6 Hz, 1H), 2.44-2.36 (m, 4H), 2.47 (s, 4H), 2.56 (q, J=10.7, 7.0 Hz, 1H), 3.26 (s, 3H), 3.77-3.63 (m, 8H), 3.91-3.79 (m, 3H), 3.99 (dd, J=10.6, 5.1 Hz, 1H), 4.60-4.28 (m, 5H), 6.11 (d, J=1.3 Hz, 1H), 6.61 (s, 2H), 7.29-7.23 (m, 1H), 7.39-7.31 (m, 4H), 7.43-7.40 (m, 2H), 7.46 (d, J=8.3 Hz, 2H), 7.61 (s, 4H), 7.65 (s, 2H), 7.94 (d, J=8.1 Hz, 1H), 8.90 (s, 1H). ¹³C NMR (126 MHz, CD₃OD) δ 14.39, 17.59, 25.48, 25.58, 34.65, 35.49, 36.48, 42.35, 43.27, 56.10, 57.89, 59.62, 66.87, 67.85, 70.04, 97.18, 120.11, 121.86, 126.60, 127.63, 128.23, 128.76, 128.93, 128.96, 130.08, 132.03, 133.57, 135.37, 135.63, 138.65, 147.45, 151.58, 153.00, 153.93, 160.27, 170.86, 170.99, 172.41, 173.44. HRMS (ESI+): m/z calcd for C₄₇H₅₈N₉P₆S: 876.4226 [M+H]⁺; found: 876.4234, [M+H]⁺.

### (2S,4R)-1-((S)-13-(tert-butyl)-2-(6-methyl-2-((4-(2-phenylacetamido)phenyl)amino)pyrimidin-4-yl)-11-oxo-5,8-dioxa-2,12-diazatetradecan-14-oyl)-4-hydroxy-N-(4-(4-methylthiazol-5-yl)benzyl)pyrrolidine-2-carboxamide (8) (i.e., Compound NUCC-0226583)

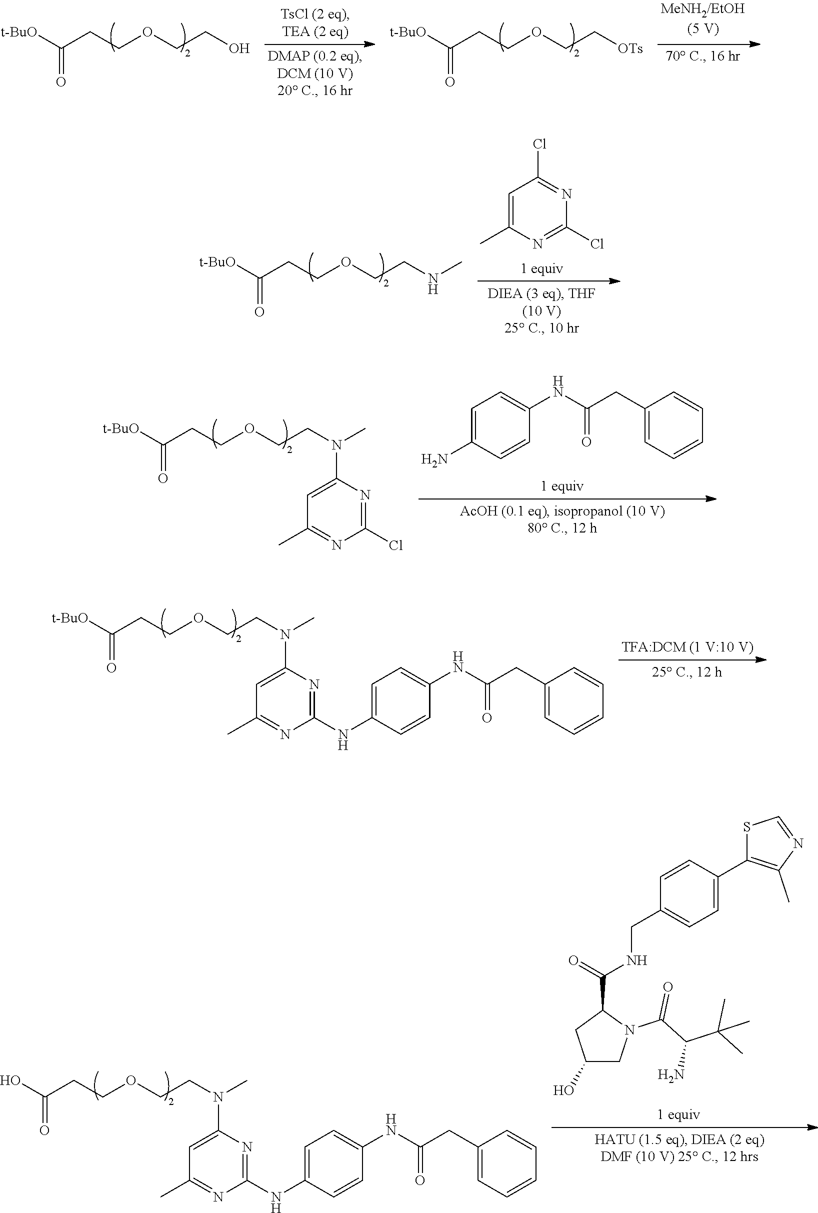

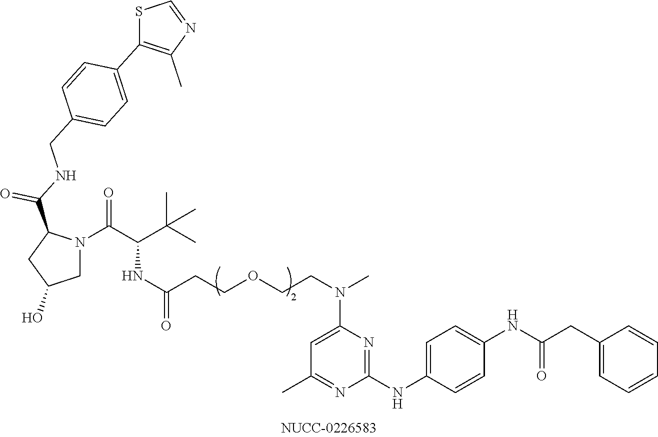

A mixture of tert-butyl 3-(2-(2-hydroxyethoxy)ethoxy)propanoate (3 g, 12.8 mmol), 4-methylbenzene-1-sulfonyl chloride (4.88 g, 25.6 mmol), triethylamine (2.59 g, 25.6 mmol) and N,N-dimethylpyridin-4-amine (312 mg, 2.56 mmol) in dichloromethane (30 mL) was degassed and purged with N₂ for 3 times, and then the mixture was stirred at 20° C. for 12 h under N₂ atmosphere. The reaction mixture was diluted with water (250 mL) and then extracted with ethyl acetate (150 mL×3). The combined organic layers were washed with aqueous sodium chloride, dried over Na₂SO₄, filtered and concentrated under reduced pressure to give a residue. The residue was purified by column chromatography on silica gel (eluted with Petroleum ether to ethyl acetate: EA=50:1 to 0:100) to give tert-butyl 3-(2-(2-(tosyloxy)ethoxy)ethoxy)propanoate, 29 (3.5 g, yield 70%) as colorless oil. ¹H NMR (400 MHz, CHLOROFORM-d) δ 1.44-1.45 (m, 9H), 2.45 (s, 3H), 2.48 (t, J=6.50 Hz, 2H), 3.52-3.58 (m, 4H), 3.65-3.70 (m, 4H), 4.13-4.18 (m, 2H), 7.35 (d, J=8.16 Hz, 2H), 7.80 (d, J=8.38 Hz, 2H).

A mixture of tert-butyl 3-(2-(2-(tosyloxy)ethoxy)ethoxy)propanoate, 29 (3.5 g, 9.01 mmol) and MeNH₂ (4 M in EtOH, 2.25 mL) was degassed and purged with N₂ for 3 times, then stirred at 20° C. for 12 h under N₂ atmosphere. The reaction was cooled to 20° C. and concentrated to give tert-butyl 3-(2-(2-(methylamino)ethoxy)ethoxy)propanoate, 30 (2 g, yield 89%) as a colorless oil. ¹H NMR (400 MHz, CHLOROFORM-d) δ 1.37-1.49 (m, 9H), 2.44-2.52 (m, 3H), 3.01-3.10 (m, 2H), 3.50-3.82 (m, 10H).

A mixture of tert-butyl 3-(2-(2-(methylamino)ethoxy)ethoxy)propanoate (4 g, 16.2 mmol), DIPEA (6.27 g, 48.5 mmol), 2,4-dichloro-6-methyl-pyrimidine, 14 (2.64 g, 16.2 mmol) in THF (40 mL) was degassed and purged with N₂ for 3 times, then stirred at 20° C. for 12 h under N₂ atmosphere. The reaction mixture was diluted with water (250 mL), and then extracted with ethyl acetate (150 mL×3). The combined organic layers were washed with aqueous sodium chloride, dried over Na₂SO₄, filtered and concentrated under reduced pressure to give a residue. The residue was purified by column chromatography on silica gel (eluted with petroleum ether ethyl acetate=50:1 to 0:100) to give tert-butyl 3-(2-(2-((2-chloro-6-methylpyrimidin-4-yl)(methyl)amino)ethoxy)ethoxy)propanoate, 31 (2.5 g, yield 41%) as a colorless oil. ¹H NMR (400 MHz, CHLOROFORM-d) δ 1.44 (s, 9H), 2.33 (s, 3H), 2.48 (t, J=6.47 Hz, 2H), 3.10 (br s, 3H), 3.53-3.60 (m, 4H), 3.63-3.82 (m, 6H), 6.19 (br s, 1H).

A mixture of tert-butyl 3-(2-(2-((2-chloro-6-methylpyrimidin-4-yl)(methyl)amino)ethoxy)ethoxy)propanoate, 31 (1.50 g, 4.01 mmol), acetic acid (120 mg, 2.01 mmol) and N-(4-aminophenyl)-2-phenyl-acetamide (907 mg, 4.01 mmol) in isopropanol (20 mL) was degassed and purged with N₂ for 3 times, then stirred at 80° C. for 12 h under N₂ atmosphere. The reaction was cooled to 20° C., filtered, and concentrated under reduced pressure to give a residue which was washed with isopropanol (50 mL) to give tert-butyl 3-(2-(2-(methyl(6-methyl-2-((4-(2-phenylacetamido)phenyl)amino)pyrimidin-4-yl)amino)ethoxy)ethoxy)propanoate, 36 (1.3 g, yield 54%) as a yellow solid. ¹H NMR (400 MHz, CHLOROFORM-d): δ 1.41-1.46 (m, 9H), 2.34-2.41 (m, 3H), 3.17-3.23 (m, 3H), 3.48-3.72 (m, 9H), 3.76-3.83 (m, 3H), 5.83-6.00 (m, 1H), 7.24-7.30 (m, 1H), 7.31-7.37 (m, 3H), 7.37-7.45 (m, 3H), 7.49-7.58 (m, 2H), 8.42-8.63 (m, 1H), 10.05-10.16 (m, 1H), 13.40-13.55 (m, 1H).

A mixture of tert-butyl 3-(2-(2-(methyl(6-methyl-2-((4-(2-phenylacetamido)phenyl)amino)pyrimidin-4-yl)amino)ethoxy)ethoxy)propanoate, 36 (0.8 g, 1.42 mmol) and trifluoroacetic acid (1 mL) in dichloromethane (10 mL) was degassed and purged with N₂ three times, then stirred at 25° C. for 12 h under N₂ atmosphere. The reaction was concentrated under reduced pressure to give 3-(2-(2-(methyl(6-methyl-2-((4-(2-phenylacetamido)phenyl)amino)pyrimidin-4-yl)amino)ethoxy)ethoxy)propanoic acid, 41 (500 mg, yield 62%) as a yellow solid. ¹H NMR (400 MHz, T=273+80K, DMSO-d6) δ 2.33 (s, 3H), 2.41 (t, J=6.28 Hz, 2H), 3.19 (s, 3H), 3.49 (br d, J=4.63 Hz, 4H), 3.57-3.69 (m, 6H), 3.72-3.82 (m, 2H), 7.21-7.26 (m, 1H), 7.28-7.38 (m, 4H), 7.46 (br d, J=8.38 Hz, 2H), 7.63 (d, J=8.82 Hz, 2H), 10.12 (s, 1H), 10.33 (s, 1H).

A mixture of (2S,4R)-1-((S)-2-amino-3,3-dimethylbutanoyl)-4-hydroxy-N-(4-(4-methylthiazol-5-yl)benzyl)pyrrolidine-2-carboxamide ((S,R,S)-AHPC, CAS 1448297-52-6 from Bide Pharmatech Ltd.) (140 mg, 325 μmol), 3-(2-(2-(methyl(6-methyl-2-((4-(2-phenylacetamido)phenyl)amino)pyrimidin-4-yl)amino)ethoxy)ethoxy)propanoic acid, 41 (150 mg, 295 μmol), HATU (168 mg, 443 μmol) and DIPEA (76.4 mg, 591 μmol,) in N,N-dimethylformamide (2 mL) was degassed and purged with N₂ three times, then stirred at 25° C. for 12 h under N₂ atmosphere. The reaction was concentrated under reduced pressure to give a residue. The residue was purified by Prep-HPLC (Gilson 281 semi-preparative HPLC system; Mobile phase: A: TFA/H₂O=0.075% v/v; B: acetonitrile; Column: Phenomenex luna C18 100×40 mm×5 μm; Flow rate: 50 mL/min; Monitoring wavelength: 220&254 nm) to give (2S,4R)-1-((S)-13-(tert-butyl)-2-(6-methyl-2-((4-(2-phenylacetamido)phenyl)amino)pyrimidin-4-yl)-11-oxo-5,8-dioxa-2,12-diazatetradecan-14-oyl)-4-hydroxy-N-(4-(4-methylthiazol-5-yl)benzyl)pyrrolidine-2-carboxamide (8, 35 mg, yield 12%) as a white solid. ¹H NMR (500 MHz, DMSO) δ 0.94 (s, 9H), 1.91 (ddd, J=4.5, 8.7, 13.1 Hz, 1H), 2.05 (dt, J=5.0, 9.5 Hz, 1H), 2.24-2.39 (m, 4H), 2.45 (s, 3H), 3.18 (m, 3H), 3.37-3.83 (m, 11H), 4.23 (dd, J=5.5, 15.9 Hz, 1H), 4.37 (q, J=3.2 Hz, 1H), 4.40-4.49 (m, 2H), 4.57 (d, J=9.3 Hz, 1H), 6.46 (m, 1H), 7.26 (dtt, J=2.3, 4.3, 6.1 Hz, 1H), 7.34 (d, J=6.5 Hz, 4H), 7.38-7.54 (m, 6H), 7.64 (dd, J=5.5, 8.8 Hz, 2H), 7.97 (d, J=9.3 Hz, 1H), 8.63 (t, J=6.1 Hz, 1H), 9.01 (s, 1H), 10.27 (s, 1H), 10.34 (d, J=10.1 Hz, 1H), 12.92 (s, 1H). HRMS (ESI+): m/z calcd for C₄₉H₆₂N₉O₇S: 920.4488 [M+H]⁺; found: 920.4503, [M+H]⁺.

### (2S,4R)-1-((S)-16-(tert-butyl)-2-(6-methyl-2-((4-(2-phenylacetamido)phenyl)amino)pyrimidin-4-yl)-14-oxo-5,8,11-trioxa-2,15-diazaheptadecan-17-oyl)-4-hydroxy-N-(4-(4-methylthiazol-5-yl)benzyl)pyrrolidine-2-carboxamide (9) (Lie., Compound NUCC-0226542)

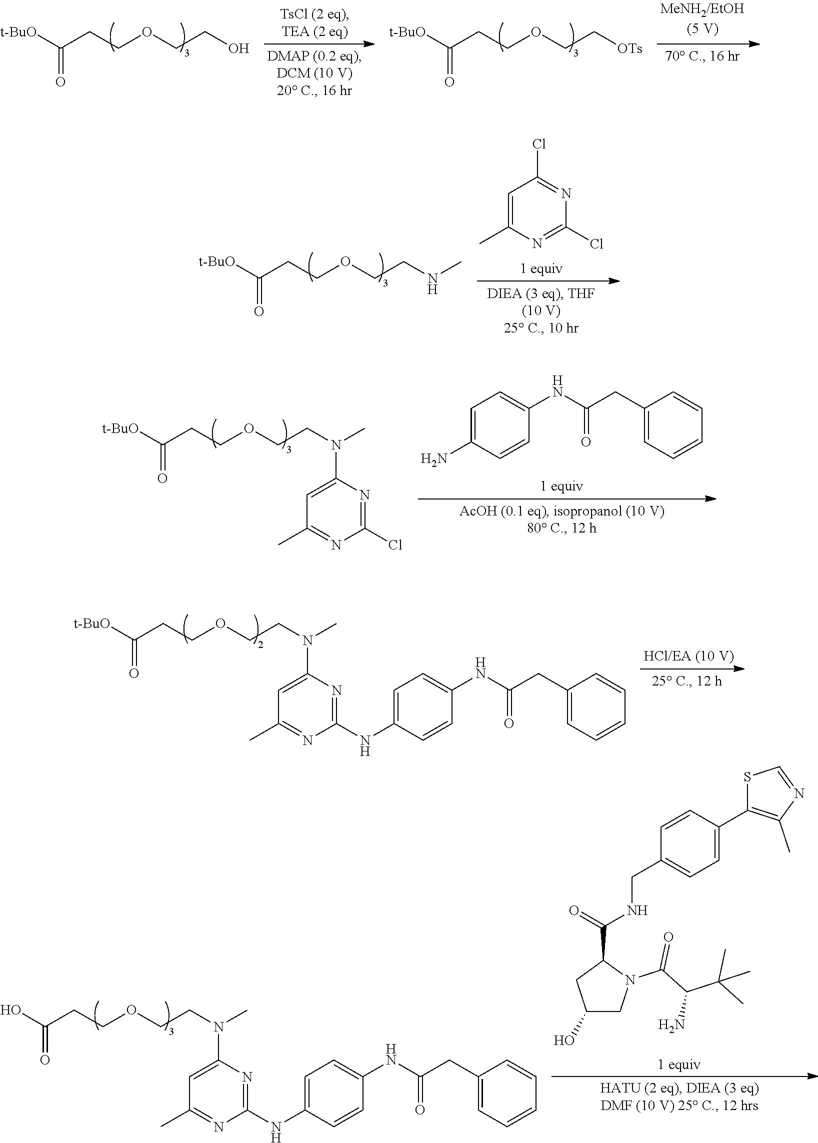

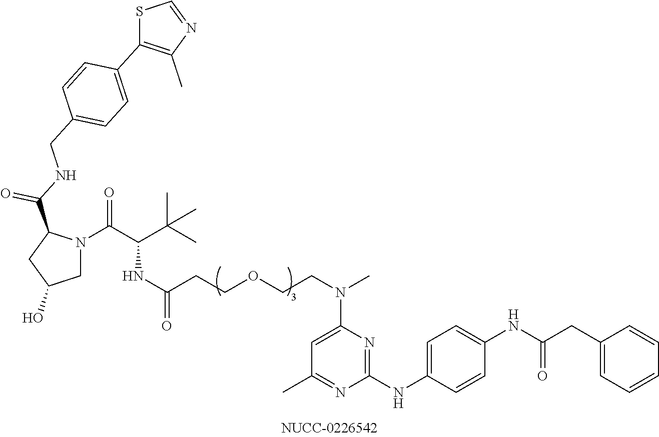

To a solution of tert-butyl 3-(2-(2-(2-hydroxyethoxy)ethoxy)ethoxy)propanoate (5 g, 17.96 mmol, 1 eq) in CH₂Cl₂ (50 mL) was added DMAP (0.44 g, 3.6 mmol, 0.2 eq), TEA (3.64 g, 35.93 mmol, 5.00 mL, 2 eq) and 4-methylbenzene-1-sulfonyl chloride (6.85 g, 35.93 mmol, 2 eq) in one portion at 0° C. The reaction was stirred at 20° C. for 16 h under nitrogen atmosphere. TLC (DCM: MeOH=10:1) showed starting material was consumed and new spots formed. The reaction was extracted with dichloromethane (3×50 mL) and the organic phases were combined and washed with brine (2×50 mL). The organic phase was dried over magnesium sulfate and concentrated to get the crude product. The crude product was purified by column chromatography on silica gel (eluted by Petroleum ether/Ethyl acetate=20/1 to 3/1) to afford tert-butyl 3-(2-(2-(2-(tosyloxy)ethoxy)ethoxy)ethoxy)propanoate (5.5 g, yield 70.79%) as a yellow oil. ¹H NMR: (400 MHz, CDCl₃) δ 1.45 (s, 9H), 2.46 (s, 3H), 2.53-2.48 (m, 2H), 3.59 (d, J=5.5 Hz, 8H), 3.75-3.67 (m, 4H), 4.21-4.11 (m, 2H), 7.35 (d, J=8.1 Hz, 2H), 7.81 (d, J=8.3 Hz, 2H)

To a mixture of tert-butyl 3-(2-(2-(2-(tosyloxy)ethoxy)ethoxy)ethoxy)propanoate (5 g, 11.56 mmol, 1 eq) in THF (50 mL) was added methanamine (23.93 g, 254.32 mmol, 33% purity, 22 eq) at 25° C. The mixture was stirred at 70° C. for 16 h. TLC (Petroleum ether: Ethyl acetate=1:1) showed the starting material was consumed. The organic phase was concentrated under reduced pressure to get tert-butyl 5,8,11-trioxa-2-azatetradecan-14-oate (4.8 g, yield 89.57%) as light yellow oil which was used in the next step directly without further purification.

To a solution of tert-butyl 5,8,11-trioxa-2-azatetradecan-14-oate (4.27 g, 9.20 mmol, 1.5 eq) and DIPEA (7.14 g, 55.20 mmol, 3.0 eq) in tetrahydrofuran (50 mL) was added 2,4-dichloro-6-methyl-pyrimidine, 14 (1 g, 6.13 mmol, 1 eq) at 20° C. under N₂. After addition, the reaction was stirred at 25° C. for 10 hours. The reaction was concentrated and purified by column chromatography on silica gel (petro ether: ethyl acetate=100:1 to 40:100) to give tert-butyl 2-(2-chloro-6-methylpyrimidin-4-yl)-5,8,11-trioxa-2-azatetradecan-14-oate, 32 (1.2 g, yield 46.8%) as a colorless oil. ¹H NMR: (400 MHz CDCl₃) δ 1.45 (s, 9H), 2.34 (s, 3H), 2.50 (t, J=6.5 Hz, 2H), 3.11 (br s, 3H), 3.64-3.55 (m, 8H), 3.74-3.65 (m, 5H), 6.29-6.09 (m, 1H).

To a solution of tert-butyl 2-(2-chloro-6-methylpyrimidin-4-yl)-5,8,11-trioxa-2-azatetradecan-14-oate, 32 (1.13 g, 2.70 mmol, 1 eq) and N-(4-aminophenyl)-2-phenyl-acetamide (642.39 mg, 2.70 mmol, 1 eq) in isopropanol (10 mL) was added acetic acid (11.87 mg, 0.1 eq) at 0° C. under N₂. After addition, the reaction was stirred at 80° C. for 12 hours. The reaction was cooled to 25° C. and filtered to obtain tert-butyl 2-(6-methyl-2-((4-(2-phenylacetamido)phenyl)amino)pyrimidin-4-yl)-5,8,11-trioxa-2-azatetradecan-14-oate, 37 (1.6 g, yield 97.37%) as a brown solid. LCMS (ESI+): m/z 608.5 (M+H)⁺.

To a solution of tert-butyl 2-(6-methyl-2-((4-(2-phenylacetamido)phenyl)amino)pyrimidin-4-yl)-5,8,11-trioxa-2-azatetradecan-14-oate, 37 (600 mg, 987.27 μmol, 1 eq) in ethyl acetate (2 mL) was added ethyl acetate/HCl (4 M, 6 mL) and the reaction mixture was stirred at 25° C. for 12 hours. The reaction was concentrated to give 2-(6-methyl-2-((4-(2-phenylacetamido)phenyl)amino)pyrimidin-4-yl)-5,8,11-trioxa-2-azatetradecan-14-oic acid, 42 (400 mg, 73.45% yield) as a brown solid which was used without further purification. LCMS (ESI+): m/z 552.4 (M+H)⁺.

To a solution of 2-(6-methyl-2-((4-(2-phenylacetamido)phenyl)amino)pyrimidin-4-yl)-5,8,11-trioxa-2-azatetradecan-14-oic acid, 42 (100 mg, 160.11 μmol, 1 eq) and (2S,4R)-1-((S)-2-amino-3,3-dimethylbutanoyl)-4-hydroxy-N-(4-(4-methylthiazol-5-yl)benzyl)pyrrolidine-2-carboxamide ((S,R,S)-AHPC, CAS 1448297-52-6 from Bide Pharmatech Ltd.) (68.94 mg, 160.11 μmol, 1 eq) in N,N-dimethylformamide (2 mL) was added HATU (121.76 mg, 320.23 μmol, 2 eq) and DIPEA (82.7761 mg, 640.46 μmol, 4 eq) at 0° C. After addition, the reaction was stirred at 25° C. for 12 hours. LCMS showed the starting material was consumed. The reaction mixture was treated with water (3 mL) and extracted with ethyl acetate (3×3 mL). The combined organic phases were washed with brine (3 mL), dried over anhydrous sodium sulfate, filtered and concentrated under reduced pressure to obtain residue which was purified by prep-HPLC (Phenomenex Gemini-NX C18 75×30 mm×3 μm; Mobile phase: [water (0.05% NH₃H₂O+10 mM NH₄HCO₃)-MeCN]; B %: 30%-60%, 8 min) to give (2S,4R)-1-((S)-16-(tert-butyl)-2-(6-methyl-2-((4-(2-phenylacetamido)phenyl)amino)pyrimidin-4-yl)-14-oxo-5,8,11-trioxa-2,15-diazaheptadecan-17-oyl)-4-hydroxy-N-(4-(4-methylthiazol-5-yl)benzyl)pyrrolidine-2-carboxamide, (9, 20 mg, yield 12.93%) as a white solid. ¹H NMR: (400 MHz, DMSO-d₆) δ 0.93 (s, 9H), 1.90 (ddd, J=12.84, 8.49, 4.53 Hz, 1H), 1.98-2.08 (m, 1H), 2.16 (s, 3H), 2.30-2.36 (m, 1H), 2.44 (s, 3H), 2.53-2.57 (m, 1H), 3.02 (s, 3H), 3.43-3.51 (m, 8H), 3.54-3.70 (m, 10H), 4.21 (dd, J=15.85, 5.36 Hz, 1H), 4.35 (br s, 1H), 4.38-4.48 (m, 2H), 4.55 (d, J=9.42 Hz, 1H), 5.12 (d, J=3.58 Hz, 1H), 5.95 (s, 1H), 7.20-7.26 (m, 1H), 7.29-7.34 (m, 4H), 7.36-7.45 (m, 6H), 7.64 (d, J=8.94 Hz, 2H), 7.91 (d, J=9.42 Hz, 1H), 8.56 (t, J=6.02 Hz, 1H), 8.90 (s, 1H), 8.97 (s, 1H), 9.96 (s, 1H). IRMS (ESI+): m/z calcd for C₅₁H₆₆N₉O₈S: 964.4750 [M+H]⁺; found: 964.4755, [M+H]⁺.

### (2S,4R)-1-((S)-19-(tert-butyl)-2-(6-methyl-2-((4-(2-phenylacetamido)phenyl)amino)pyrimidin-4-yl)-17-oxo-5,8,11,14-tetraoxa-2,18-diazaicosan-20-oyl)-4-hydroxy-N-(4-(4-methylthiazol-5-yl)benzyl)pyrrolidine-2-carboxamide (10) (i.e., Compound NUCC-0226584)

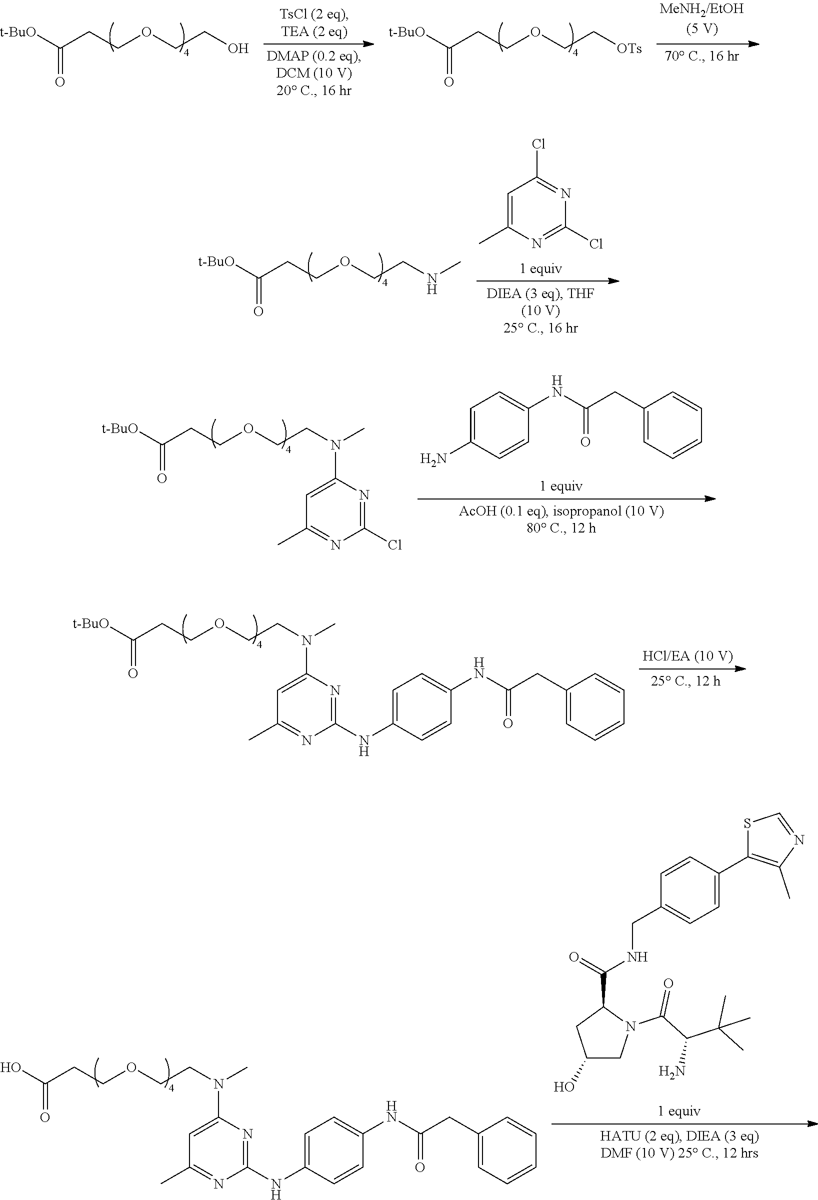

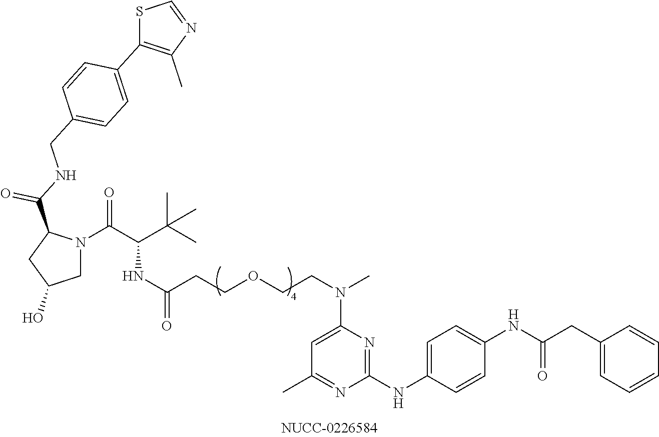

To a solution of tert-butyl 1-hydroxy-3,6,9,12-tetraoxapentadecan-15-oate (2 g, 6.20 mmol) in DCM (20 mL) was added TosCl (2.37 g, 12.41 mmol), TEA (1.26 g, 12.41 mmol) and DMAP (151.58 mg, 1.24 mmol) at 20° C. The reaction was stirred at 20° C. for 16 h. TLC showed the reaction was complete. The reaction was quenched with H₂O (30 mL) and extracted with DCM (3×30 mL). The organic layer was dried over sodium sulfate and filtered. The filtrate was concentrated to give a crude product, which was purified by column on silica gel (eluted with PE:EA=100:1 to 5:1) to give tert-butyl 1-(tosyloxy)-3,6,9,12-tetraoxapentadecan-15-oate, 34 (1.8 g, yield 60.88%) as a colorless oil. ¹H NMR: (400 MHz, CDCl₃) δ 1.44 (s, 9H), 2.45 (s, 3H), 2.49 (t, J=6.60 Hz, 2H), 3.58 (s, 4H), 3.61 (br s, 8H), 3.67-3.73 (m, 4H), 4.16 (t, J=4.83 Hz, 2H), 7.34 (d, J=7.95 Hz, 2H), 7.80 (d, J=8.19 Hz, 2H).

A mixture of tert-butyl 1-(tosyloxy)-3,6,9,12-tetraoxapentadecan-15-oate, 34 (1.8 g, 3.78 mmol) in MeNH₂/EtOH (10 mL) was stirred at 70° C. for 16 h. TLC (Petroleum ether: Ethyl acetate=1:1) showed the starting material was consumed. The organic phase was concentrated under reduced pressure to give tert-butyl 5,8,11,14-tetraoxa-2-azaheptadecan-17-oate, 35 (1.8 g, yield 92.35%, 65% purity) as a light yellow oil which was used for the next step directly without further purification. ¹H NMR: (400 MHz, CDCl₃) δ 1.44-1.47 (m, 9H), 2.38 (s, 3H), 2.50 (t, J=6.36 Hz, 2H), 2.75-2.79 (m, 2H), 3.18-3.30 (m, 2H), 3.64-3.68 (m, 8H), 3.72 (br t, J=6.24 Hz, 4H), 3.85-3.96 (m, 2H).

To a solution of tert-butyl 5,8,11,14-tetraoxa-2-azaheptadecan-17-oate (1.8 g, 3.49 mmol, 65% purity) and DIPEA (901.60 mg, 6.98 mmol) in THF (18 mL) was added 2,4-dichloro-6-methyl-pyrimidine, 14 (379.04 mg, 2.33 mmol) at 20° C. The reaction was stirred at 20° C. for 16 h. The reaction was concentrated to a residue which was purified by column chromatography on silica gel (petro ether: ethyl acetate=100:1 to 1:2) to give tert-butyl 2-(2-chloro-6-methylpyrimidin-4-yl)-5,8,11,14-tetraoxa-2-azaheptadecan-17-oate, 33 (1.1 g, yield 81.92%, 80% purity) as a colorless oil. ¹H NMR: (400 MHz CDCl₃) δ 1.37 (s, 9H), 2.22-2.30 (m, 3H), 2.39-2.46 (m, 2H), 3.03 (br s, 3H), 3.50-3.57 (m, 12H), 3.57-3.70 (m, 6H), 6.13 (br s, 1H).

To a solution of tert-butyl 2-(2-chloro-6-methylpyrimidin-4-yl)-5,8,11,14-tetraoxa-2-azaheptadecan-17-oate, 33 (1.1 g, 2.38 mmol) and AcOH (14.30 mg, 238.11 μmol) in isopropanol (11 mL) was added N-(4-aminophenyl)-2-phenyl-acetamide (538.77 mg, 2.38 mmol) at 20° C. The reaction was stirred at 80° C. for 12 h. LCMS showed the reaction was complete and the desired compound was detected. The reaction was diluted with H₂O (20 mL) and extracted with ethyl acetate (3×25 mL). The organic layer was concentrated under reduced pressure to give tert-butyl 2-(6-methyl-2-((4-(2-phenylacetamido)phenyl)amino)pyrimidin-4-yl)-5,8,11,14-tetraoxa-2-azaheptadecan-17-oate, 38 (0.5 g, yield 32.22%) as a white solid. ¹H NMR: (400 MHz CDCl₃) δ 1.42 (d, J=1.32 Hz, 9H), 2.24 (s, 3H), 3.08 (s, 3H), 3.51-3.73 (m, 21H), 5.79 (s, 1H), 7.29-7.38 (m, 6H), 7.45-7.56 (m, 3H), 8.35-8.51 (m, 1H).

To a solution of tert-butyl 2-(6-methyl-2-((4-(2-phenylacetamido)phenyl)amino)pyrimidin-4-yl)-5,8,11,14-tetraoxa-2-azaheptadecan-17-oate, 38 (0.5 g, 767.12 μmol) in ethyl acetate (5 mL) was added ethyl acetate/HCl (4 M, 5 mL) at 25° C. and the reaction was stirred at 25° C. for 12 h. The reaction was concentrated to give 2-(6-methyl-2-((4-(2-phenylacetamido)phenyl)amino)pyrimidin-4-yl)-5,8,11,14-tetraoxa-2-azaheptadecan-17-oic acid, 43 (0.2 g, yield 43.77%) as a brown solid which was used without further purification.

To a solution of 2-(6-methyl-2-((4-(2-phenylacetamido)phenyl)amino)pyrimidin-4-yl)-5,8,11,14-tetraoxa-2-azaheptadecan-17-oic acid, 43 (0.1 g, 167.87 μmol) and (2S,4R)-1-((S)-2-amino-3,3-dimethylbutanoyl)-4-hydroxy-N-(4-(4-methylthiazol-5-yl)benzyl)pyrrolidine-2-carboxamide ((S,R,S)-AHPC, CAS 1448297-52-6 from Bide Pharmatech Ltd.) (72.28 mg, 167.87 μmol) in DMF (1 mL) was added HATU (127.66 mg, 335.75 μmol) and DIPEA (65.09 mg, 503.62 μmol) at 25° C. The reaction was stirred at 25° C. for 12 h. LCMS showed the starting material was consumed and LCMS indicated desired product. The reaction mixture was treated with water (3 mL) and extracted with ethyl acetate (3×3 mL). The combined organic phases were washed with brine (3 mL), dried over anhydrous sodium sulfate, filtered and concentrated under reduced pressure to obtain a residue which was purified by prep-HPLC (Phenomenex Gemini-NX C18 75×30 mm×3 μm; Mobile phase: [water (0.05% NH₃H₂O+10 mM NH₄HCO₃)-MeCN]; B %: 30%-60%, 8 min) to give (2S,4R)-1-((S)-19-(tert-butyl)-2-(6-methyl-2-((4-(2-phenylacetamido)phenyl)amino)pyrimidin-4-yl)-17-oxo-5,8,11,14-tetraoxa-2,18-diazaicosan-20-oyl)-4-hydroxy-N-(4-(4-methylthiazol-5-yl)benzyl)pyrrolidine-2-carboxamide, (10, 48 mg, yield 28.36%) as a white solid. ¹H NMR: (400 MHz, DMSO-d₆) δ 0.93 (s, 9H), 1.90 (ddd, J=12.78, 8.44, 4.58 Hz, 1H), 1.98-2.08 (m, 1H), 2.16 (s, 3H), 2.25-2.42 (m, 2H), 2.44 (s, 3H), 2.52-2.56 (m, 1H), 3.02 (br s, 3H), 3.46 (s, 7H), 3.49 (br d, J=0.98 Hz, 4H), 3.53-3.72 (m, 10H), 4.22 (dd, J=15.77, 5.38 Hz, 1H), 4.35 (br s, 1H), 4.39-4.48 (m, 2H), 4.55 (d, J=9.41 Hz, 1H), 5.12 (d, J=3.42 Hz, 1H), 5.95 (s, 1H), 7.20-7.27 (m, 1H), 7.28-7.35 (m, 4H), 7.36-7.47 (m, 6H), 7.64 (d, J=8.80 Hz, 2H), 7.91 (d, J=9.41 Hz, 1H), 8.56 (br t, J=5.93 Hz, 1H), 8.90 (s, 1H), 8.97 (s, 1H), 9.96 (s, 1H). HRMS (ESI+): m/z calcd for C₅₃H₇₀N₉O₉S: 1008.5012 [M+H]⁺; found: 1008.5015, [M+H]⁺.

### (2S,4R)-1-((S)-22-(tert-buty)-2-(6-methyl-2-((4-(2-phenylacetamido)phenyl)amino)pyrimidin-4-yl)-20-oxo-5,8,11,14,17-pentaoxa-2,21-diazatricosan-23-oyl)-4-hydroxy-N-(4-(4-methylthiazol-5-yl)benzyl)pyrrolidine-2-carboxamide (11) (i.e., Compound NUCC-0226543). P_{GP}-6₄,C₃

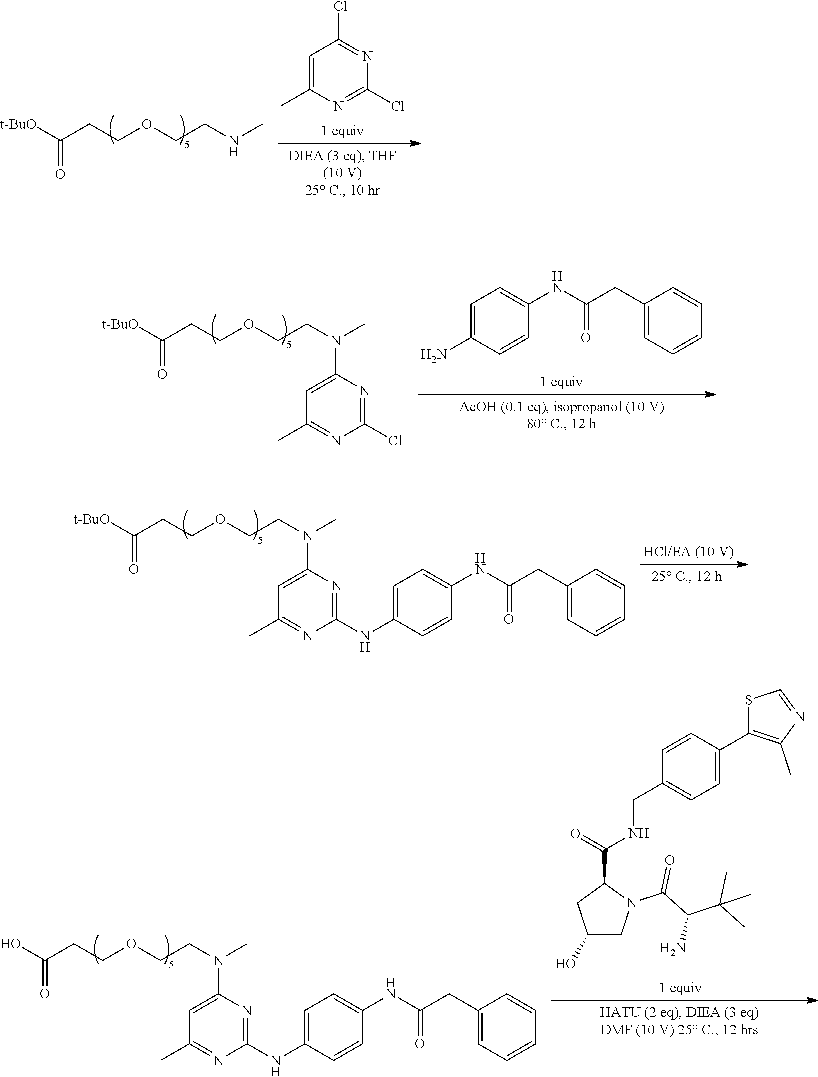

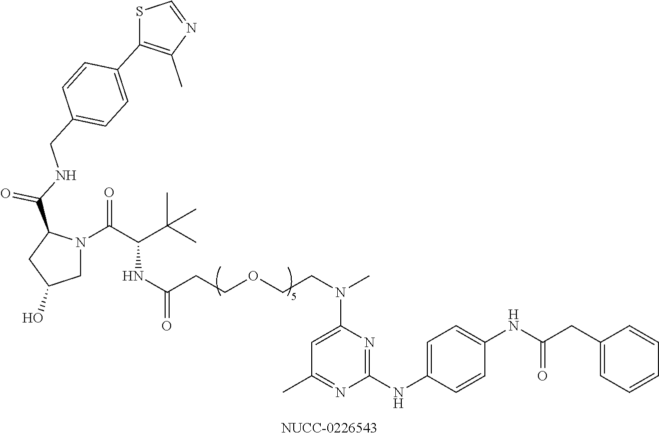

To a solution of tert-butyl 5,8,11,14,17-pentaoxa-2-azaicosan-20-oate, (3.5 g, 9.22 mmol, 1.5 eq) and DIPEA (2.38 g, 18.45 mmol, 3 eq) in tetrahydrofuran (30 mL) was added 2,4-dichloro-6-methyl-pyrimidine (14, 2.06 g, 6.15 mmol, 1 eq) at 20° C. under N₂. After addition, the reaction was stirred at 20° C. for 16 hrs. The reaction was concentrated and purified by column chromatography on silica gel (petro ether: ethyl acetate=100:1 to 40:100) to give tert-butyl 2-(2-chloro-6-methylpyrimidin-4-yl)-5,8,11,14,17-pentaoxa-2-azaicosan-20-oate (34, 1 g, yield 32.1%) as a colorless oil. ¹H NMR: (400 MHz CDCl₃) δ 1.45 (s, 9H) 2.35 (s, 3H) 2.51 (t, J=6.56 Hz, 2H) 3.11 (br s, 3H) 3.60-3.66 (m, 17H) 3.68 (br d, J=4.65 Hz, 2H) 3.71 (br t, J=6.62 Hz, 3H) 6.20 (br s, 1H).

To a solution of tert-butyl 2-(2-chloro-6-methylpyrimidin-4-yl)-5,8,11,14,17-pentaoxa-2-azaicosan-20-oate, 34 (1 g, 1.98 mmol, 1 eq) and N-(4-aminophenyl)-2-phenyl-acetamide (447.15 mg, 1.98 mmol, 1 eq) in isopropanol (10 mL) was added acetic acid (11.87 mg, 0.1 eq) at 0° C. under N₂. After addition, the reaction was stirred at 80° C. for 12 hrs. Then the reaction was cooled to 25° C. and filtered to obtain tert-butyl 2-(6-methyl-2-((4-(2-phenylacetamido)phenyl)amino)pyrimidin-4-yl)-5,8,11,14,17-pentaoxa-2-azaicosan-20-oate (39, 800 mg, yield 58.18%) as a brown solid. ¹H NMR: (400 MHz CDCl₃) δ 1.45 (s, 9H), 2.25 (s, 3H), 2.49 (t, J=6.56 Hz, 2H), 3.08 (s, 3H), 3.56-3.67 (m, 19H), 3.67-3.75 (m, 6H), 5.82 (s, 1H), 6.84 (br s, 1H), 7.32-7.42 (m, 7H), 7.52 (d, J=8.94 Hz, 2H).

To a solution of tert-butyl 2-(6-methyl-2-((4-(2-phenylacetamido)phenyl)amino)pyrimidin-4-yl)-5,8,11,14,17-pentaoxa-2-azaicosan-20-oate (39, 300 mg, 431.13 μmol, 1 eq) in ethyl acetate (1 mL) was added HCl/EtOAc (4 M, 5 mL) and the reaction was stirred at 25° C. for 2 hrs. The reaction was concentrated to give 2-(6-methyl-2-((4-(2-phenylacetamido)phenyl)amino)pyrimidin-4-yl)-5,8,11,14,17-pentaoxa-2-azaicosan-20-oic acid (44, 200 mg, yield 72.51%) as a brown solid which was used without further purification. LCMS(ESI+): m/z 640.4 (M+H)⁺.

To a solution of 2-(6-methyl-2-((4-(2-phenylacetamido)phenyl)amino)pyrimidin-4-yl)-5,8,11,14,17-pentaoxa-2-azaicosan-20-oic acid (44, 67.30 mg, 156.31 μmol, 1 eq) and (2S,4R)-1-((S)-2-amino-3,3-dimethylbutanoyl)-4-hydroxy-N-(4-(4-methylthiazol-5-yl)benzyl)pyrrolidine-2-carboxamide ((S,R,S)-AHPC, CAS 1448297-52-6 from Bide Pharmatech Ltd.) (100 mg, 156.31 μmol, 1 eq) in DMF (1 mL) was added HATU (118.87 mg, 312.63 μmol, 2 eq) and DIPEA (60.61 mg, 468.94 μmol, 81.68 μL, 3 eq) at 0° C. After addition, the reaction was stirred at 25° C. for 12 hrs. TLC (dichloromethane:methanol=10:1) showed the starting material was consumed. The reaction mixture was treated water (3 mL) and extracted with ethyl acetate (3×3 mL). The combined organic phases were washed with brine (3 mL), dried over anhydrous sodium sulfate, filtered and concentrated under reduced pressure. The residue was purified by prep-TLC (SiO₂, dichloromethane: methanol=10:1) to give (2S,4R)-1-((S)-25-(tert-butyl)-2-(6-methyl-2-((4-(2-phenylacetamido)phenyl)amino)pyrimidin-4-yl)-23-oxo-5,8,11,14,17,20-hexaoxa-2,24-diazahexacosan-26-oyl)-4-hydroxy-N-(4-(4-methylthiazol-5-yl)benzyl)pyrrolidine-2-carboxamide (11, 20 mg, yield 12.16%) as a white solid. ¹H NMR: (400 MHz, DMSO-d₆) δ 0.93 (s, 9H), 1.90 (ddd, J=12.83, 8.33, 4.49 Hz, 1H), 1.98-2.07 (m, 1H), 2.16 (s, 3H), 2.33-2.39 (m, 1H), 2.44 (s, 2H), 3.02 (s, 3H), 3.30 (s, 1H), 3.45-3.50 (m, 17H), 3.56-3.60 (m, 6H), 3.62-3.70 (m, 4H), 4.21 (dd, J=15.68, 5.37 Hz, 1H), 4.34 (br s, 1H), 4.39-4.47 (m, 2H), 4.55 (d, J=9.21 Hz, 1H), 5.13 (d, J=3.51 Hz, 1H), 5.95 (s, 1H), 7.23 (td, J=5.75, 2.30 Hz, 1H), 7.30-7.34 (m, 4H), 7.41 (t, J=8.77 Hz, 6H), 7.64 (d, J=8.77 Hz, 2H), 7.92 (d, J=9.43 Hz, 1H), 8.57 (t, J=5.92 Hz, 1H), 8.90 (s, 1H), 8.98 (s, 1H), 9.96 (s, 1H). ¹³C NMR (126 MHz, DMSO) δ 16.39, 24.19, 26.75, 35.82, 36.03, 36.70, 38.39, 42.06, 43.69, 48.84, 56.71, 56.85, 59.14, 67.37, 68.43, 69.31, 69.88, 70.11, 70.16, 70.18, 70.23, 70.29, 93.17, 118.88, 119.87, 126.89, 127.83, 128.50, 128.71, 129.07, 129.29, 129.49, 130.05, 131.60, 132.65, 136.68, 137.55, 139.94, 148.14, 151.93, 159.54, 162.87, 165.43, 168.87, 169.95, 170.38, 172.39. HRMS (ESI+): m/z calcd for C₅₅H₇₄N₉O₁₀S: 1052.5274 [M+H]⁺; found: 1052.5279 [M+H]⁺.

### (2S,4S)-1-((S)-22-(tert-butyl)-2-(6-methyl-2-((4-(2-phenylacetamido)phenyl)amino)pyrimidin-4-yl)-20-oxo-5,8,11,14,17-pentaoxa-2,21-diazatricosan-23-oyl)-4-hydroxy-N-(4-(4-methylthiazol-5-yl)benzyl)pyrrolidine-2-carboxamide (13) (i.e., compound NUCC-0227102)

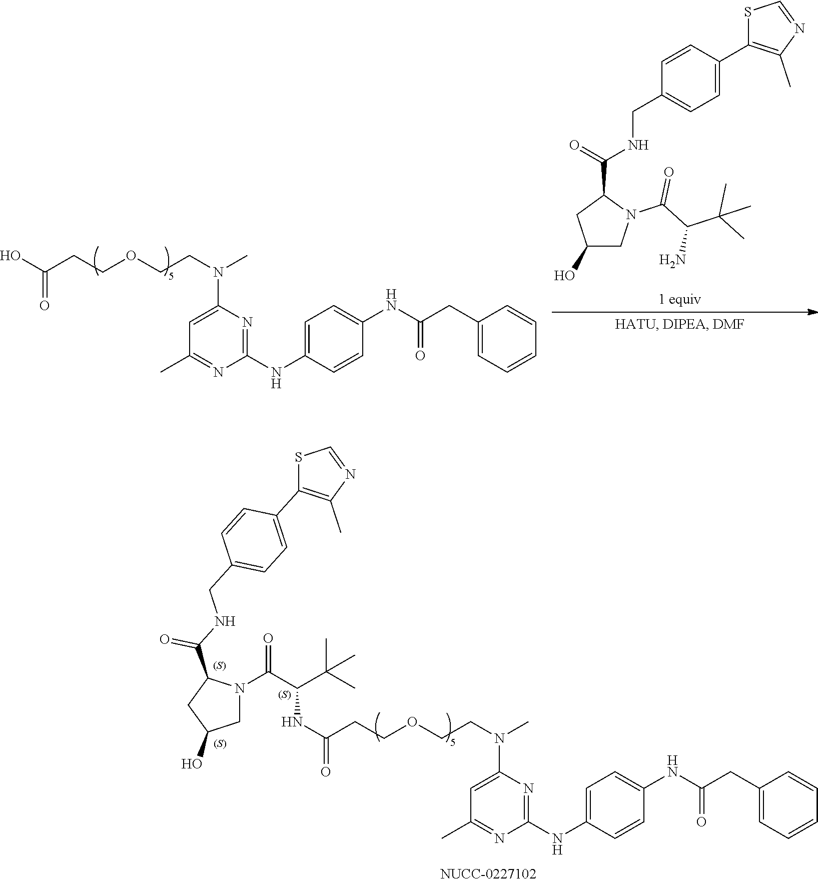

To a vial containing 2-(6-methyl-2-((4-(2-phenylacetamido)phenyl)amino)pyrimidin-4-yl)-5,8,11,14,17-pentaoxa-2-azaicosan-20-oic acid (44, 30.0 mg, 1 Eq, 46.9 μmol) was added DMF (1 mL) after which the mixture was cooled to zero degrees and HATU (26.7 mg, 1.5 Eq, 70.3 μmol) was added. Then DIPEA (18.2 mg, 24.5 μL, 3 Eq, 141 μmol) was added and the reaction was stirred for 10 min after which (2S,4S)-1-((S)-2-amino-3,3-dimethylbutanoyl)-4-hydroxy-N-(4-(4-methylthiazol-5-yl)benzyl)pyrrolidine-2-carboxamide hydrochloride (21.9 mg, 1 Eq, 46.9 μmol, VHL negative control (S,S,S)-AHPC hydrochloride from MedChemExpress; Cat #HY-125845A) was added and the reaction was stirred in the ice bath for 10 min then at room temperature for 2 h after which the mixture was directly purified by RP HPLC (Phenomenex Gemini-NX C18, 110 Å, 150×21.2 mm; 5 μm. Eluting with a gradient of 15% to 90% acetonitrile in water with 0.1% TFA, a flow rate of 20 mL/min, gradient time of 30 min, and detection wavelength of 200 nm) to give (2S,4S)-1-((S)-22-(tert-butyl)-2-(6-methyl-2-((4-(2-phenylacetamido)phenyl)amino)pyrimidin-4-yl)-20-oxo-5,8,11,14,17-pentaoxa-2,21-diazatricosan-23-oyl)-4-hydroxy-N-(4-(4-methylthiazol-5-yl)benzyl)pyrrolidine-2-carboxamide (13, 27 mg, 49%). ¹H NMR (500 MHz, CD₃OD) δ 1.04 (s, 9H), 1.98 (dt, J=13.1, 4.6 Hz, 1H), 2.42-2.38 (m, 3H), 2.47-2.40 (m, 1H), 2.49 (s, 3H), 2.56 (ddd, J=15.0, 7.5, 5.3 Hz, 1H), 3.28 (s, 3H), 3.62-3.52 (m, 15H), 3.77-3.64 (m, 7H), 3.86 (t, J=5.1 Hz, 2H), 4.02 (dd, J=10.6, 5.1 Hz, 1H), 4.39 (dt, J=9.9, 5.3 Hz, 2H), 4.58-4.46 (m, 3H), 6.12 (d, J=1.1 Hz, 1H), 7.30-7.24 (m, 1H), 7.39-7.32 (m, 4H), 7.44-7.39 (m, 2H), 7.49-7.45 (m, 2H), 7.62 (s, 4H), 7.95 (d, J=8.3 Hz, 1H), 8.97 (s, 1H). ¹³C NMR (126 MHz, CD₃OD) δ 14.40, 17.60, 25.51, 25.62, 34.79, 35.68, 35.88, 36.51, 42.36, 43.28, 50.13, 56.10, 57.74, 59.58, 66.81, 69.93, 70.02, 70.04, 70.06, 70.08, 70.13, 70.18, 97.15, 120.11, 121.89, 126.61, 127.61, 127.78, 128.24, 128.76, 128.93, 128.96, 130.05, 132.07, 133.58, 135.40, 135.67, 138.68, 147.45, 151.60, 152.92, 160.29, 170.82, 170.95, 172.54, 172.63, 173.48. HRMS (ESI+): m/z calcd for C₅₅H₇₄N₉O₁₀S: 1052.5274 [M+H]⁺; found: 1052.5276, [M+H]⁺.

### Molecular Modeling

The TG2 crystal structure 4PYG was used for docking studies. The protein was prepared using the default parameters of the Schrödinger workflow implemented in Maestro. The protein preparation module was used to complete missing atoms, relieve torsional inconsistencies, fix protonation states at pH=7.4, and the protein was minimized using the OPLS3 force field. A receptor grid was then generated centered on Leu102, which was assessed to be the approximate middle of the fibronectin-binding site (residues 88-106), and the grid extended 25 Å in each direction around this residue to encompass the FN-binding site. The MT4 structure was prepared for docking using the ligand preparation module to produce the low-energy conformation at pH=7.4. Docking was performed using the standard parameters of the XP level of Schrödinger Glide, and the low-energy conformation (among 10 poses) was evaluated.

### Cell Lines, Reagents and Antibodies

SKOV3 cells were purchased from ATCC. OVCAR5 cells were obtained from Dr. Marcus Peter at Northwestern University. Cells were maintained at 37° C. in an environment of 5% CO2 and 100% humidity. The media used for culturing the cell lines are included in Table 1. Cells lines used in all experiments were at low-passages and were confirmed to be pathogen and mycoplasma-free by Charles River Animal Diagnostic Services. Cell lines were authenticated by IDEXX BioAnalytics with short tandem repeat (STR) profiling. MG132 was obtained from SelleckChem. Anti-TG2 antibody (cat #: MAB3839-I-100UG) was purchased from Millipore Sigma, GAPDH antibody (cat #: H86504M) was purchased from Meridian Bioscience.

### Western Blotting

Briefly, SKOV3 and OVCAR5 cells were seeded on 6 cm dishes and treated with different concentrations of compounds at different time points. Proteins were extracted using RIPA buffer and quantified using the Bradford assay. After gel electrophoresis and transfer, PDVF membranes were blocked with TBS-Tween 5% BSA for one hour and incubated with primary antibodies 20 (1:1000 dilution, overnight at 4° C.). After incubation with the secondary antibody (anti rabbit/mouse-horseradish peroxidase 1:1000 dilution) for 1 h at room temperature, signal was developed using SuperSignal West Pico PLUS Chemiluminescent Substrate (Thermo Fisher Scientific cat #: 34580) and captured with an ImageQuant LAS 4000 machine. To detect additional proteins, membranes were treated with Restore Western Blot Stripping Buffer (Thermo Fisher Scientific cat #: 21059), blocked, and then incubated with primary antibody.

### Cell Migration

For the scratch assay, confluent 6-well plates were scratched with a pipette tip, washed and treated with compounds or vehicle. The plate was photographed at set time points and the area of wound closure was quantified with ImageJ. Cell migration was assessed by using Transwell inserts (8 μm pore size, Merck-Millipore, Watford, UK). Briefly, 15,000 cells were seeded in the upper chambers of trans-wells in serum-free media containing compounds or control, while media containing 10% serum was added to the lower chamber. After 18 hours, cells invaded through the transwells were fixed with warm 4% PFA for 20 min at room temperature and stained with crystal violet for 3 min. The membranes were removed, mounted on cover slides and cells were counted using a microscope at 40× magnification.

### Cell Viability Assay

Cells seeded in 96 well dishes were treated with compounds at different concentrations (0.1, 0.3, 1, 3, 10, 30 μM) for 24 hrs. Media was replaced after 24 hours and cells were allowed to growth for another 48 hours. The numbers of viable cells were quantified by using the CCK-8 assay (ApexBio) following the manufacturer's directions. Absorbance was measured by a spectrophotometer at 495 nm and used estimate the viability of the cells.

### Solid Phase Adhesion Assay

Briefly, wells were coated with 10 μg/ml fibronectin diluted in PBS for one hour. 30,000 cells pretreated with compounds for 6 hours were seeded and allowed to attach for 30 minutes. After washing with PBS to remove unattached cells, cells adhering to coated wells were quantified by using the CCK-8 assay (ApexBio), following the manufacturer's protocol. Relative absorbance measured at 496 nm was used to estimate the relative number of the cell attached to fibronectin coated wells.

### Isothermal Titration Calorimetry (ITC)

Sample Preparation. Protein dilutions in PBS with 1% DMSO were prepared from stock solutions of 108 mM TG240 in PBS. The protein dilutions were initially equilibrated on ice for 5 min, then for 20 min at 30° C. The solutions were filtered using a 0.22 μM Millipore centrifugal device just prior to insertion in the ITC cell. The solubility of the protein solutions was confirmed independently by DLS (data not shown), and the concentration of each solution (after filtration) was measured with a NanoDrop 2000 Instrument.

For each compound, dilutions were prepared from stocks in DMSO; specifically, 40 mM stock solutions were used for compound 11 and compound 7, and a 20 mM stock solution for MT4 due to its lower solubility than the PROTAC compounds. All compound dilutions were made in PBS, and the final DMSO concentration in each solution was carefully adjusted to 1%.

ITC Experiments. All ITC experiments were performed on an Affinity Low Volume instrument (Waters/TA Instruments) equipped with a fixed 198 μL gold cell. In each experiment, 320 μL of protein solution was loaded into the ITC cell, and 200 μL of the corresponding compound was in the syringe. The data were acquired using ITCRun data acquisition software. The incremental ITC experiments consisted of multiple 2 μL injections at 50-350-second intervals with a stirring speed of 100 revolutions per minute (rpm). The Auto Equilibrate function allowed for the equilibration of the baseline to a peak-to-peak standard deviation of less than 12 nW. For each compound, separate control experiments were performed to evaluate the heat of dilution. The control experiments were set with identical parameters but performed with reaction buffer instead of protein in the ITC cell and consisted of fewer (6-10) injections.

Data Analysis. ITC data were analyzed with Nano Analyze Software (Waters/TA Instruments) using the “independent sites” model after correction for heats of dilution. Of note, because of limitations in the solubility of the compounds and the protein's sensitivity to higher (>1%) concentrations of DMSO, these experiments were performed at c values lower than 1.

### Statistical Analysis

Data are presented as means±SD with *p<0.05, **p<0.01, ***p<0.001, and ****p<0.0001. Statistical significance was determined by using two-tailed Student's t-test (Prism 8, Graphpad Software). P values less than 0.05 were considered statistically significant. The number of biological replicates in each experiment is indicated by n in every figure caption.

### ABBREVIATIONS

AcOH, acetic acid; ATCC, American Type Culture Collection; CRBN, cereblon; CSC, cancer stem cell; DHI, bromodihy-droisoxazole; DIPEA, diisopropylamine; DMF, dimethylforma-mide; DMSO, dimethylsulfoxide; EGFR, epidermal growth factor receptor; EMT, epithelial-mesenchymal transition; ESI, electrospray ionization; EtOAc, ethyl acetate; FN, fibronectin; GDP, Guanosine diphosphate; GTP, Guanosine triphosphate; HATU, 2-(7-azabenzotriazol-1-yl)-N,N,N′,N′-tetramethyluro-nium hexafluorophosphate; HCl, hydrochloric acid; HRMS, high-resolution mass spectrometry; IMSERC, Integrated Molecular Structure Education and Research Center; IPA, isopropanol; ITC, isothermal calorimetry; LCMS (LC/MS), liquid chromatography/mass spectrometry; NMR, nuclear magnetic resonance; OC, ovarian cancer; PDB, protein databank; PEG, poly(ethylene glycol); PK, pharmacokinetics; POI, protein of interest; PROTAC, PROteolysis-TArgeting Chimera; SAR, structure-activity relationship; STR, short tandem repeat; TG2, tissue transglutaminase; THF, tetrahy-drofuran; TLC, thin-layer chromatography; TOF, time-of-flight; UPLC, ultra-high pressure liquid chromatography; VHL, von Hippel-Lindau.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

(1) Huang, X.; Lin, T.; Gu, J.; Zhang, L.; Roth, J. A.; Stephens, L. C.; Yu, Y.; Liu, J.; Fang, B. Combined TRAIL and Bax gene therapy prolonged survival in mice with ovarian cancer xenograft. Gene Ther. 2002, 9, 1379-1386.
(2) Stephens, P.; Grenard, P.; Aeschlimann, P.; Langley, M.; Blain, E.; Errington, R.; Kipling, D.; Thomas, D.; Aeschlimann, D. Crosslinking and G-protein functions of transglutaminase 2 contribute differentially to fibroblast wound healing responses. J. Cell Sci. 2004, 117, 3389-3403.
(3) Fesus, L.; Piacentini, M. Transglutaminase 2: an enigmatic enzyme with diverse functions. Trends Biochem. Sci. 2002, 27, 534-539.
(4) Akimov, S. S.; Belkin, A. M. Cell surface tissue transglutaminase is involved in adhesion and migration of monocytic cells on fibronectin. Blood 2001, 98, 1567-1576.
(5) Akimov, S. S.; Krylov, D.; Fleischman, L. F.; Belkin, A. M. Tissue transglutaminase is an integrin-binding adhesion coreceptor for fibronectin. J. Cell Biol. 2000, 148, 825-838.
(6) Verderio, E. A.; Telci, D.; Okoye, A.; Melino, G.; Griffin, M. A novel RGD-independent cel adhesion pathway mediated by fibronectin-bound tissue transglutaminase rescues cells from anoikis, J. Biol. Chem. 2003, 278, 42604-42614.
(7) Zemskov, E. A.; Loukinova, E.; Mikhailenko, 1.; Coleman, R. A.; Strickland, D. K.; Belkin, A. M. Regulation of platelet-derived growth factor receptor function by integrin-associated cell surface trans-glutaminase. J. Biol. Chem. 2009, 284, 16693-16703.
(8) Zemskov, E. A.; Mikhailenko, I.; Smith, E. P.; Belkin, A. M. Tissue transglutaminase promotes PDGF/PDGFR-mediated signaling and responses in vascular smooth muscle cells. J. Cell Physiol. 2012, 227, 2089-2096.
(9) Condello, S.; Sima, L. E.; Ivan, C.; Cardenas, H.; Schiltz, G. E.; Mishra, R. K.; Matei, D. Tissue transglutaminase regulates interactions between ovarian cancer stem cells and the tumor niche. Cancer Res. 2018, 78, 2990-3001.
(10) Facchiano, F.; Facchiano, A.; Facchiano, A. M. The role of transglutaminase-2 and its substrates in human diseases. Front. Biosci. 2006, 11, 1758-1773.
(11) Yuan, Z. Q.; Sun, M.; Feldman, R. I.; Wang, G.; Ma, X.; Jiang, C.; Coppola, D.; Nicosia, S. V.; Cheng, J. Q. Frequent activation of AKT2 and induction of apoptosis by inhibition of phosphoinositide-3-OH kinase/Akt pathway in human ovarian cancer. Oncogene 2000, 19, 2324-2330.
(12) Yi, M. C.; Khosla, C. Thiol-Disulfide Exchange Reactions in the Mammalian Extracellular Environment. Annu. Rev. Chem. Biomol. Eng. 2016, 7, 197-222.
(13) Palanski, B. A.; Khosla, C. Cystamine and Disulfiram Inhibit Human Transglutaminase 2 via an Oxidative Mechanism. Biochemistry 2018, 57, 3359-3363.
(14) Akimov, S. S.; Belkin, A. M. Cell-surface transglutaminase promotes fibronectin assembly via interaction with the gelatin-binding domain of fibronectin: a role in TGFbeta-dependent matrix deposition. J. Cell Sci. 2001, 114, 2989-3000.
(15) Belkin, A. M.; Tsurupa, G.; Zemskov, E.; Veklich, Y.; Weisel, J. W.; Medved, L. Transglutaminase-mediated oligomerization of the fibrin(ogen) alphaC domains promotes integrin-dependent cell adhesion and signaling. Blood 2005, 105, 3561-3568.
(16) Kaartinen, M. T.; El-Maadawy, S.; Rasanen, N. H.; McKee, M. D. Tissue transglutaminase and its substrates in bone. J. Bone Miner. Res. 2002, 17, 2161-2173.
(17) Aeschlimann, D.; Paulsson, M. Cross-linking of laminin-nidogen complexes by tissue transglutaminase. A novel mechanism for basement membrane stabilization. J. Biol. Chem. 1991,266, 15308-15317.
(18) Jones, R. A.; Kotsakis, P.; Johnson, T. S.; Chau, D. Y.; Ali, S.; Melino, G.; Griffin, M. Matrix changes induced by transglutaminase 2 lead to inhibition of angiogenesis and tumor growth. Cell Death Differ. 2005, 13, 1442-1453.
(19) Satpathy, M.; Cao, L.; Pincheira, R.; Emerson, R.; Bigsby, R.; Nakshatri, H.; Matei, D. Enhanced peritoneal ovarian tumor dissemination by tissue transglutaminase. Cancer Res. 2007, 67, 7194-7202.
(20) Shao, M.; Cao, L.; Shen, C.; Satpathy, M.; Chelladurai, B.; Bigsby, R. M.; Nakshatri, H.; Matei, D. Epithelial-to-mesenchymal transition and ovarian tumor progression induced by tissue trans-glutaminase. Cancer Res. 2009, 69, 9192-9201.
(21) Iacobuzio-Donahue, C. A.; Ashfaq, R.; Maitra, A.; Adsay, N. V.; Shen-Ong, G. L.; Berg, K.; Hollingsworth, M. A.; Cameron, J. L.; Yeo, C. J.; Kern, S. E.; Goggins, M.; Hruban, R. H. Highly expressed genes in pancreatic ductal adenocarcinomas: a comprehensive characterization and comparison of the transcription profiles obtained from three major technologies. Cancer Res. 2003, 63, 8614-8622.
(22) Martinet, N.; Bonnard, L.; Regnault, V.; Picard, E.; Burke, L.; Siat, J.; Grosdidier, G.; Martinet, Y.; Vignaud, J. M. In vivo transglutaminase type 1 expression in normal lung, preinvasive bronchial lesions, and lung cancer. Am. J. Respir. Cell Mol. Biol. 2003, 28, 428-435.
(23) Grigoriev, M. Y.; Suspitsin, E. N.; Togo, A. V.; Pozharisski, K. M.; Ivanova, O. A.; Nardacci, R.; Falasca, L.; Piacentini, M.; Imyanitov, E. N.; Hanson, K. P. Tissue transglutaminase expression in breast carcinomas. J. Exp. Clin. Cancer Res. 2001, 20, 265-268.
(24) Derynck, R.; Zhang, Y. E. Smad-dependent and Smad-independent pathways in TGF-beta family signalling. Nature 2003, 425, 577-584.
(25) Hwang, J. Y.; Mangala, L. S.; Fok, J. Y.; Lin, Y. G.; Merritt, W. M.; Spannuth, W. A.; Nick, A. M.; Fiterman, D. J.; Vivas-Mejia, P. E.; Deavers, M. T.; Coleman, R. L.; Lopez-Berestein, G.; Mehta, K.; Sood, A. K. Clinical and biological significance of tissue transglutaminase in ovarian carcinoma. Cancer Res. 2008, 68, 5849-5858.
(26) Verma, A.; Wang, H.; Manavathi, B.; Fok, J. Y.; Mann, A. P.; Kumar, R.; Mehta, K. Increased expression of tissue transglutaminase in pancreatic ductal adenocarcinoma and its implications in drug resistance and metastasis. Cancer Res. 2006, 66, 10525-10533.
(27) Jeong, J. H.; Cho, B. C.; Shim, H. S.; Kim, H. R.; Lim, S. M.; Kim, S. K.; Chung, K. Y.; Islam, S. M.; Song, J. J.; Kim, S. Y.; Kim, J. H. Transglutaminase 2 expression predicts progression free survival in non-small cell lung cancer patients treated with epidermal growth factor receptor tyrosine kinase inhibitor. J. Korean Med. Sci. 2013, 28, 1005-1014.
(28) Mehta, K.; Lopez-Berestein, G.; Moore, W. T.; Davies, P. J. Interferon-gamma requires serum retinoids to promote the expression of tissue transglutaminase in cultured human blood monocytes. J. Immunol. 1985, 134, 2053-2056.
(29) Cao, L.; Petrusca, D. N.; Satpathy, M.; Nakshatri, H.; Petrache, I.; Matei, D. Tissue Transglutaminase Protects Epithelial Ovarian Cancer Cells from Cisplatin Induced Apoptosis by Promoting Cell Survival Signaling. Carcinogenesis 2008, 29, 1893-1900.
(30) Mann, A. P.; Verma, A.; Sethi, G.; Manavathi, B.; Wang, H.; Fok, J. Y.; Kunnumakkara, A. B.; Kumar, R.; Aggarwal, B. B.; Mehta, K. Overexpression of Tissue Transglutaminase Leads to Constitutive Activation of Nuclear Factor-{kappa}B in Cancer Cells: Delineation of a Novel Pathway. Cancer Res. 2006, 66, 8788-8795.
(31) Cao, L.; Shao, M.; Schilder, J.; Guise, T.; Mohammad, K. S.; Matei, D. Tissue transglutaminase links TGF-β, epithelial to mesenchymal transition and a stem cell phenotype in ovarian cancer. Oncogene 2012, 31, 2521-2534.
(32) Verma, A.; Guha, S.; Wang, H.; Fok, J. Y.; Koul, D.; Abbruzzese, J.; Mehta, K. Tissue transglutaminase regulates focal adhesion kinase/AKT activation by modulating PTEN expression in pancreatic cancer cells. Clin. Cancer Res. 2008, 14, 1997-2005.
(33) Condello, S.; Cao, L.; Matei, D. Tissue transglutaminase regulates beta-catenin signaling through a c-Src-dependent mechanism. FASEB J. 2013, DOI: 10.1096/fj.12-222620.
(34) Verma, A.; Guha, S.; Diagaradjane, P.; Kunnumakkara, A. B.; Sanguino, A. M.; Lopez-Berestein, G.; Sood, A. K.; Aggarwal, B. B.; Krishnan, S.; Gelovani, J. G.; Mehta, K. Therapeutic significance of elevated tissue transglutaminase expression in pancreatic cancer. Clin. Cancer Res. 2008, 14, 2476-2483.
(35) Singh, U. S.; Kunar, M. T.; Kao, Y. L.; Baker, K. M. Role of transglutaminase II in retinoic acid-induced activation of RhoA-associated kinase-2. EMBO J. 2001, 20, 2413-2423.
(36) Kerr, C.; Szmacinski, H.; Fisher, M. L.; Nance, B.; Lakowicz, J. R.; Akbar, A.; Keillor, J. W.; Lok Wong, T.; Godoy-Ruiz, R.; Toth, E. A.; Weber, D. J.; Eckert, R. L. Transamidase site-targeted agents alter the conformation of the transglutaminase cancer stem cell survival protein to reduce GTP binding activity and cancer stem cell survival. Oncogene 2017, 36, 2981-2990.
(37) Kumar, A.; Xu, J.; Sung, B.; Kumar, S.; Yu, D.; Aggarwal, B. B.; Mehta, K. Evidence that GTP-binding domain but not catalytic domain of transglutaminase 2 is essential for epithelial-to-mesenchymal transition in mammary epithelial cells. Breast Cancer Res. 2012, 14, No. R4.
(38) Yuan, L.; Choi, K.; Khosla, C.; Zheng, X.; Higashikubo, R.; Chicoine, M. R.; Rich, K. M. Tissue transglutaminase 2 inhibition promotes cell death and chemosensitivity in glioblastomas. Mol. Cancer Ther. 2005, 4, 1293-1302.
(39) Yuan, L.; Siegel, M.; Choi, K.; Khosla, C.; Miller, C. R.; Jackson, E. N.; Piwnica-Worms, D.; Rich, K. M. Transglutaminase 2 inhibitor, KCC009, disrupts fibronectin assembly in the extracellular matrix and sensitizes orthotopic glioblastomas to chemotherapy. Oncogene 2007, 26, 2563-2573.
(40) Yakubov, B.; Chen, L.; Belkin, A. M.; Zhang, S.; Chelladurai, B.; Zhang, Z. Y.; Matei, D. Small molecule inhibitors target the tissue transglutaminase and fibronectin interaction. PLoS One 2014, 9, No. e89285.
(41) Sima, L. E.; Yakubov, B.; Zhang, S.; Condello, S.; Grigorescu, A. A.; Nwani, N. G.; Chen, L.; Schiltz, G. E.; Arvanitis, C.; Zhang, Z. Y.; Matei, D. Small Molecules Target the Interaction between Tissue Transglutaminase and Fibronectin. Mol. Cancer Ther. 2019, 18, 1057-1068.
(42) Zou, Y.; Ma, D.; Wang, Y. The PROTAC technology in drug development. Cell Biochem. Funct. 2019, 37, 21-30.
(43) Bondeson, D. P.; Smith, B. E.; Burslem, G. M.; Buhimschi, A. D.; Hines, J.; Jaime-Figueroa, S.; Wang, J.; Hamman, B. D.; Ishchenko, A.; Crews, C. M. Lessons in PROTAC design from selective degradation with a promiscuous warhead. Cell Chem. Biol. 2018, 25, 78-87.e5.
(44) Sakamoto, K. M.; Kim, K. B.; Kumagai, A.; Mercurio, F.; Crews, C. M.; Deshaies, R. J. Protacs: chimeric molecules that target proteins to the Skpl-Cullin-F box complex for ubiquitination and degradation. Proc. Natl. Acad. Sc.i U.S.A. 2001, 98, 8554-8559.
(45) Schmidt, H. R.; Zheng, S.; Gurpinar, E.; Koehl, A.; Manglik, A.; Kruse, A. C. Crystal structure of the human sigmal receptor. Nature 2016, 532, 527-530.
(46) Gadd, M. S.; Testa, A.; Lucas, X.; Chan, K.-H.; Chen, W.; Lamont, D. J.; Zengerle, M.; Ciulli, A. Structural basis of PROTAC cooperative recognition for selective protein degradation. Nat. Chem. Biol. 2017, 13, 514-521.
(47) Neklesa, T. K.; Crews, C. M. Chemical biology: Greasy tags for protein removal. Nature 2012, 487, 308-309.
(48) Winter, G. E.; Buckley, D. L.; Paulk, J.; Roberts, J. M.; Souza, A.; Dhe-Paganon, S.; Bradner, J. E. DRUG DEVELOPMENT. Phthali-mide conjugation as a strategy for in vivo target protein degradation. Science 2015, 348, 1376-1381.
(49) Neklesa, T.; Snyder, L. B.; Willard, R. R.; Vitale, N.; Pizzano, J.; Gordon, D. A.; Bookbinder, M.; Macaluso, J.; Dong, H.; Ferraro, C.; et al. ARV-110: an oral androgen receptor PROTAC degrader for prostate cancer. J. Clin. Oncol. 2019, 37, 259.
(50) Mullard, A. Targeted protein degraders crowd into the clinic. Nat. Rev. Drug Discovery 2021, 20, 247-250.
(51) B6k6s, M.; Langley, D. R.; Crews, C. M. PROTAC targeted protein degraders: the past is prologue. Nat. Rev. Drug Discovery 2022, 21, 181-200.
(52) Burslem, G. M.; Smith, B. E.; Lai, A. C.; Jaime-Figueroa, S.; McQuaid, D. C.; Bondeson, D. P.; Toure, M.; Dong, H.; Qian, Y.; Wang, J.; Crew, A. P.; Hines, J.; Crews, C. M. The Advantages of Targeted Protein Degradation Over Inhibition: An RTK Case Study. Cell Chem. Biol. 2018, 25, 67-77.e3.
(53) Akbar, A.; McNeil, N. M. R.; Albert, M. R.; Ta, V.; Adhikary, G.; Bourgeois, K.; Eckert, R. L.; Keillor, J. W. Structure-Activity Relationships of Potent, Targeted Covalent Inhibitors That Abolish Both the Transamidation and GTP Binding Activities of Human Tissue Transglutaminase. J. Med. Chem. 2017, 60, 7910-7927.
(54) Raina, K.; Lu, J.; Qian, Y.; Altieri, M.; Gordon, D.; Rossi, A. M. K.; Wang, J.; Chen, X.; Dong, H.; Siu, K.; Winkler, J. D.; Crew, A. P.; Crews, C. M.; Coleman, K. G. PROTAC-induced BET protein degradation as a therapy for castration-resistant prostate cancer. Proc. Natl. Acad. Sci. U.S.A. 2016, 113, 7124-7129.
(55) Song, M.; Hwang, H.; Im, C. Y.; Kim, S. Y. Recent Progress in the Development of Transglutaminase 2 (TGase2) Inhibitors. J. Med. Chem. 2017, 60, 554-567.
(56) Klöck, C.; Herrera, Z.; Albertelli, M.; Khosla, C. Discovery of potent and specific dihydroisoxazole inhibitors of human trans-glutaminase 2. J. Med. Chem. 2014, 57, 9042-9064.
(57) Chrobok, N. L.; Bol, J.; Jongenelen, C. A.; Breve, J. J. P.; El Alaoui, S.; Wilhelmus, M. M. M.; Drukarch, B.; van Dam, A. M. Characterization of Transglutaminase 2 activity inhibitors in mono-cytes in vitro and their effect in a mouse model for multiple sclerosis. PLoS One 2018, 13, No. e0196433.
(58) Büchold, C.; Hils, M.; Gerlach, U.; Weber, J.; Pelzer, C.; Heil, A.; Aeschlimann, D.; Pasternack, R. Features of ZED1227: The First-In-Class Tissue Transglutaminase Inhibitor Undergoing Clinical Evalua-tion for the Treatment of Celiac Disease. Cells 2022, 11, 1667.
(59) Wityak, J.; Prime, M. E.; Brookfield, F. A.; Courtney, S. M.; Erfan, S.; Johnsen, S.; Johnson, P. D.; Li, M.; Marston, R. W.; Reed, L.; Vaidya, D.; Schaertl, S.; Pedret-Dunn, A.; Beconi, M.; Macdonald, D.; Munoz-Sanjuan, I.; Dominguez, C. SAR Development of Lysine-Based Irreversible Inhibitors of Transglutaminase 2 for Huntington's Disease. ACS Med. Chem. Lett. 2012, 3, 1024-1028.
(60) McNeil, N. M. R.; Gates, E. W. J.; Firoozi, N.; Cundy, N. J.; Leccese, J.; Eisinga, S.; Tyndall, J. D. A.; Adhikary, G.; Eckert, R. L.; Keillor, J. W. Structure-activity relationships of N-terminal variants of peptidomimetic tissue transglutaminase inhibitors. Eur. J. Med. Chem. 2022, 232, No. 114172.
(61) Wodtke, R.; Hauser, C.; Ruiz-Gomez, G.; Jackel, E.; Bauer, D.; Lohse, M.; Wong, A.; Pufe, J.; Ludwig, F. A.; Fischer, S.; Hauser, S.; Greif, D.; Pisabarro, M. T.; Pietzsch, J.; Pietsch, M.; Loser, R. N(epsilon)-Acryloyllysine Piperazides as Irreversible Inhibitors of Transglutaminase 2: Synthesis, Structure-Activity Relationships, and Pharmacokinetic Profiling. J. Med. Chem. 2018, 61, 4528-4560.
(62) Wodtke, R.; Wodtke, J.; Hauser, S.; Laube, M.; Bauer, D.; Rothe, R.; Neuber, C.; Pietsch, M.; Kopka, K.; Pietzsch, J.; Loser, R. Development of an (18)F-Labeled Irreversible Inhibitor of Trans-glutaminase 2 as Radiometric Tool for Quantitative Expression Profiling in Cells and Tissues. J. Med. Chem. 2021, 64, 3462-3478.
(63) van der Wildt, B.; Wilhelmus, M. M.; Bijkerk, J.; Haveman, L. Y.; Kooijman, E. J.; Schuit, R. C.; Bol, J. G.; Jongenelen, C. A.; Lammertsma, A. A.; Drukarch, B.; Windhorst, A. D. Development of carbon-11 labeled acryl amides for selective PET imaging of active tissue transglutaminase. Nucl. Med. Biol. 2016, 43, 232-242.
(64) Duval, E.; Case, A.; Stein, R. L.; Cuny, G. D. Structure-activity relationship study of novel tissue transglutaminase inhibitors. Bioorg. Med. Chem. Lett. 2005, 15, 1885-1889.
(65) Keillor, J. W.; Apperley, K. Y.; Akbar, A. Inhibitors of tissue transglutaminase. Trends Pharmacol. Sci. 2015, 36, 32-40.
(66) Case, A.; Stein, R. L. Kinetic analysis of the interaction of tissue transglutaminase with a nonpeptidic slow-binding inhibitor. Biochem-istry 2007, 46, 1106-1115.
(67) Pardin, C.; Pelletier, J. N.; Lubell, W. D.; Keillor, J. W. Cinnamoyl inhibitors of tissue transglutaminase. J. Org. Chem. 2008, 73, 5766-5775.
(68) Ku, B. M.; Kim, S. J.; Kim, N.; Hong, D.; Choi, Y. B.; Lee, S. H.; Gong, Y. D.; Kim, S. Y. Transglutaminase 2 inhibitor abrogates renal cell carcinoma in xenograft models. J. Cancer Res. Clin. Oncol. 2014, 140, 757-767.
(69) Khanna, M.; Chelladurai, B.; Gavini, A.; Li, L.; Shao, M.; Courtney, D.; Turchi, J. J.; Matei, D.; Meroueh, S. Targeting ovarian tumor cell adhesion mediated by tissue transglutaminase. Mol. Cancer Ther. 2011, 10, 626-636.
(70) Zengerle, M.; Chan, K. H.; Ciulli, A. Selective Small Molecule Induced Degradation of the BET Bromodomain Protein BRD4. ACS Chem. Biol. 2015, 10, 1770-1777.
(71) Bondeson, D. P.; Mares, A.; Smith, I. E.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. Catalytic in vivo protein knockdown by small-molecule PROTACs. Nat. Chem. Biol. 2015, 11, 611-617.
(72) Satpathy, M.; Shao, M.; Emerson, R.; Donner, D. B.; Matei, D. Tissue transglutaminase regulates matrix metalloproteinase-2 in ovarian cancer by modulating cAMP-response element-binding protein activity. J. Biol. Chem. 2009, 284, 15390-15399.
(73) Filippakopoulos, P.; Qi, J.; Picaud, S.; Shen, Y.; Smith, W. B.; Fedorov, O.; Morse, E. M.; Keates, T.; Hickman, T. T.; Felletar, I.; Philpott, M.; Munro, S.; McKeown, M. R.; Wang, Y.; Christie, A. L.; West, N.; Cameron, M. J.; Schwartz, B.; Heightman, T. D.; La Thangue, N.; French, C. A.; Wiest, O.; Kung, A. L.; Knapp, S.; Bradner, J. E. Selective inhibition of BET bromodomains. Nature 2010, 468, 1067-1073.
(74) Turnbull, W. B.; Daranas, A. H. On the value of c: can low affinity systems be studied by isothermal titration calorimetry? J. Am. Chem. Soc. 2003, 125, 14859-14866.

### Example 2

Additional exemplary compounds are shown in Table 2 below.

TABLE 2
Additional exemplary compounds.

| Molecule Name | Structure |
|---|---|
| NUCC-0227350 | 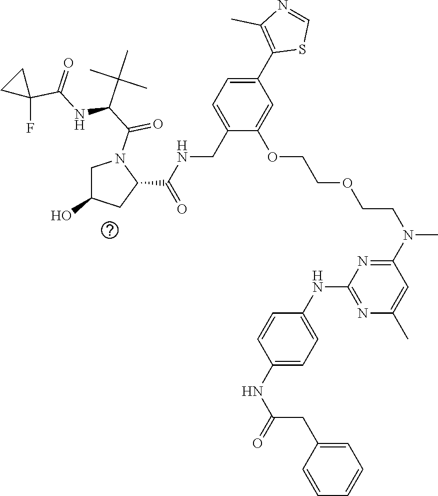 [SMILES: Cc1cc(N(C)CCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1] |
| NUCC-0227351 | 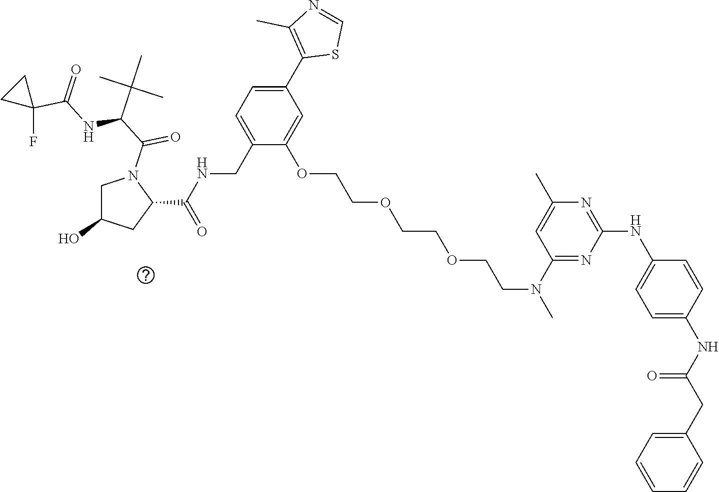 [SMILES: Cc1cc(N(C)CCOCCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1] |
| NUCC-0227352 | 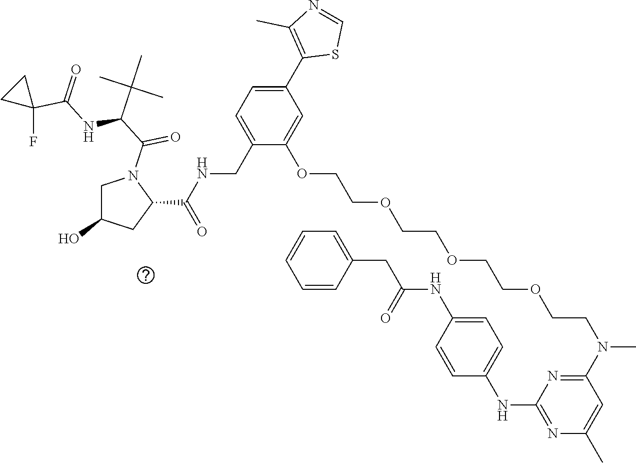 [SMILES: Cc1cc(N(C)CCOCCOCCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1] |
| NUCC-0227374 | 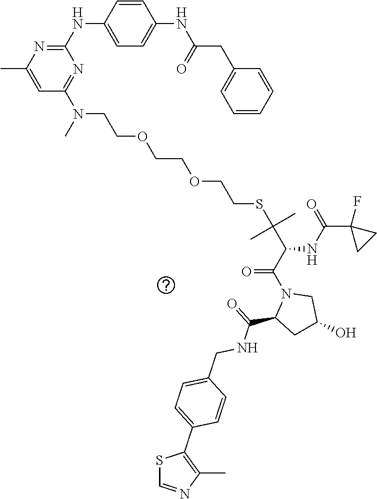 [SMILES: Cc1cc(N(C)CCOCCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1] |
| NUCC-0227404 | 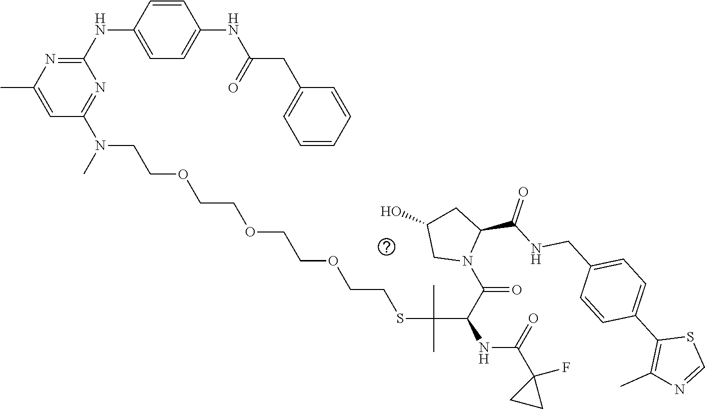 [SMILES: Cc1cc(N(C)CCOCCOCCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1] |
| NUCC-0227405 | 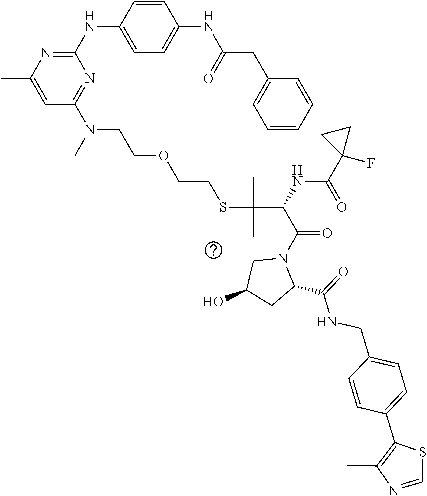 [SMILES: Cc1cc(N(C)CCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1] |
| NUCC-0228723 | 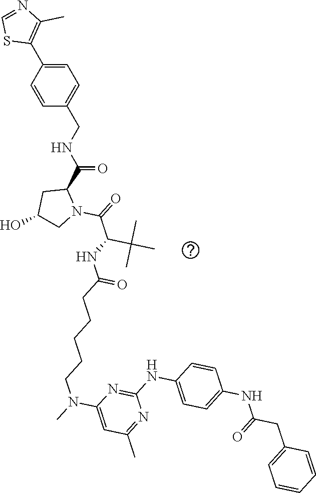 [SMILES: Cc1cc(N(C)CCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1] |
| NUCC-0228741 | 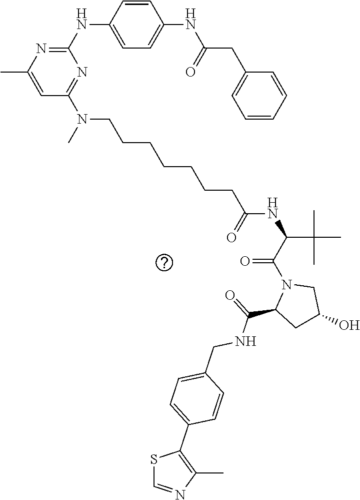 [SMILES: Cc1cc(N(C)CCCCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1] |
| NUCC-0227323 | 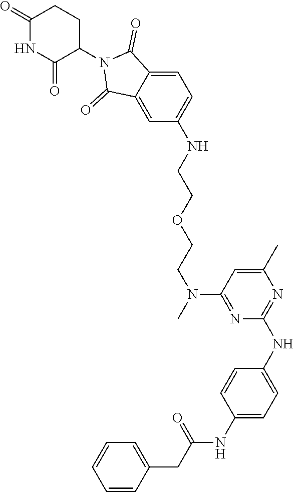 [SMILES: Cc1cc(N(C)CCOCCNc2ccc3c(c2)C(=O)N(C2CCC(=O)NC2=O)C3=O)nc(Nc2ccc(NC(=O)Cc3ccccc3)cc2)n1] |
| NUCC-0227324 | 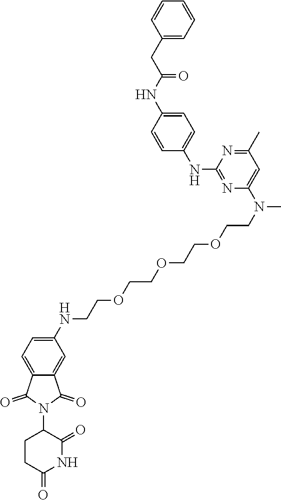 [SMILES: Cc1cc(N(C)CCOCCOCCOCCNc2ccc3c(c2)C(=O)N(C2CCC(=O)NC2=O)C3=O)nc(Nc2ccc(NC(=O)Cc3ccccc3)cc2)n1] |
| NUCC-0227325 | 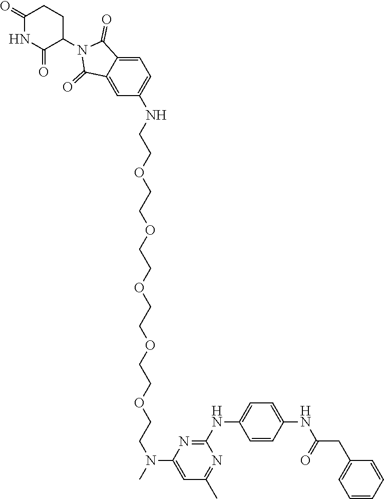 [SMILES: Cc1cc(N(C)CCOCCOCCOCCOCCOCCNc2ccc3c(c2)C(=O)N(C2CCC(=O)NC2=O)C3=O)nc(Nc2ccc(NC(=O)Cc3ccccc3)cc2)n1] |

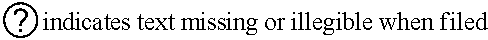

### Synthesis of compounds

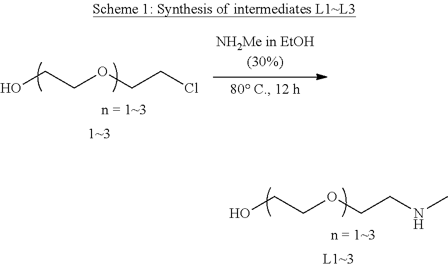

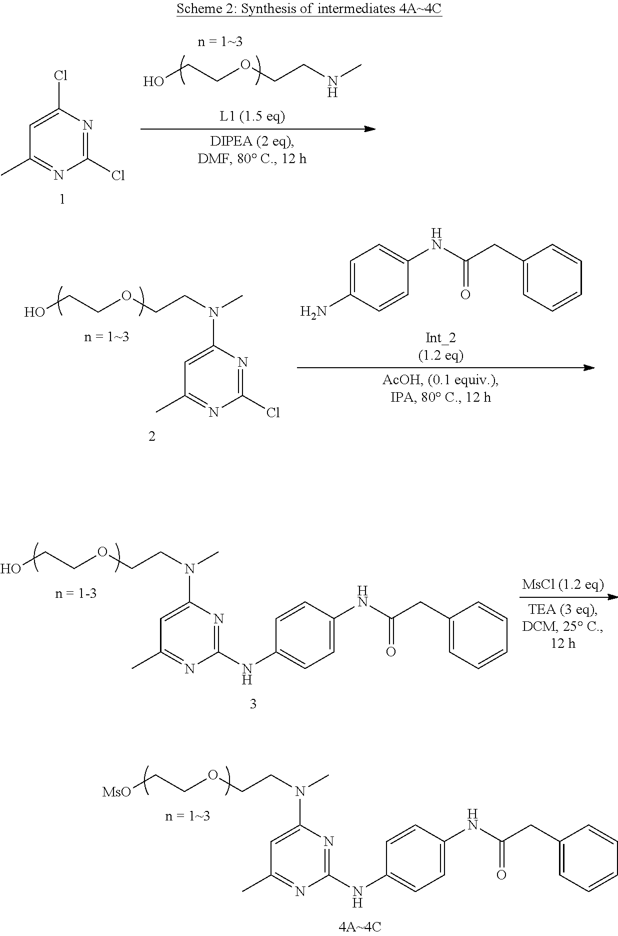

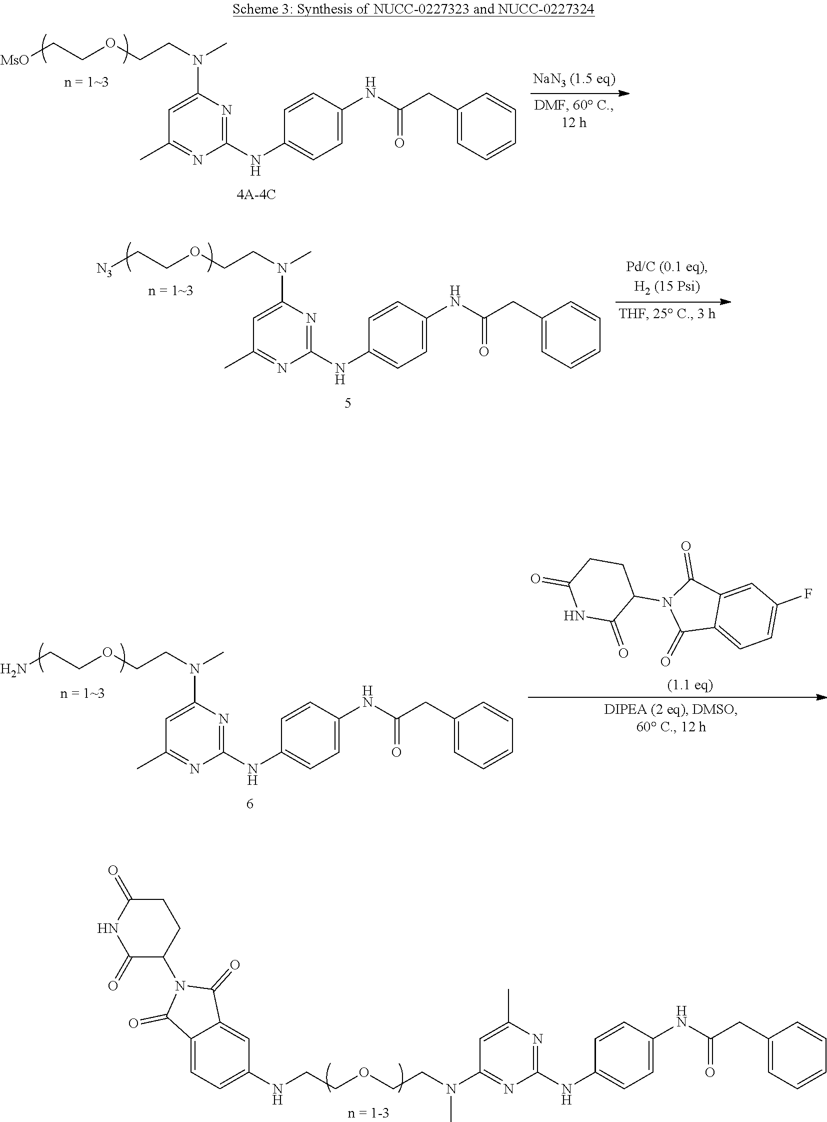

Compound NUCC-0227325 can be synthesized by using the same procedure.e

### Experimental Procedure

### General Procedure for Preparation of L1

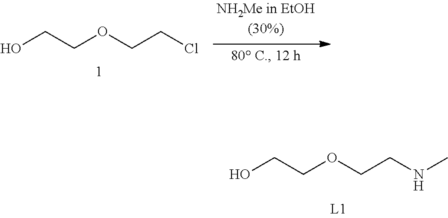

To a solution of NH₂Me in EtOH (50 mL, 30% purity) was added 2-(2-chloroethoxy)ethan-1-ol (1) (5 g, 80.28 mmol, 8.47 mL) at 20° C. Then the mixture was stirred at 80° C. for 12 h. TLC showed the reaction was completed and the desired point was detected. The mixture was concentrated to give 2-(2-(methylamino)ethoxy)ethan-1-ol (L1) (4.5 g, yield 94.08%) as colorless oil.

### General Procedure for Preparation of 2

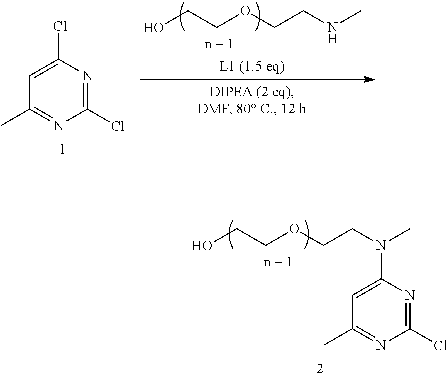

To a mixture of 2,4-dichloro-6-methylpyrimidine (1) (2.0 g, 12.27 mmol,) in DMF (20 mL) were added 2-(2-(methylamino)ethoxy)ethan-1-ol (L1) (2.19 g, 18.40 mmol) and DIPEA (3.17 g, 24.54 mmol, 4.27 mL) at 20° C. And the mixture was stirred at 80° C. for 12 h. TLC showed the reaction was completed and the desired point was detected. The reaction mixture was diluted with water (20 mL) and extracted with DCM (3×20 mL). The organic combined organic layers were washed with brine (3×20 mL) and dried over Na₂SO₄. After filtered, the filtrate was concentrated under reduced pressure to give the crude product. The crude product was purified by column chromatography on silica gel (eluted with PE: EA=100:1 to 0:1) to give 2-(2-((2-chloro-6-methylpyrimidin-4-yl)(methyl)amino)ethoxy)ethanol (2) (1.5 g, yield 28.07%) as white solid.

### General Procedure for Preparation of 3

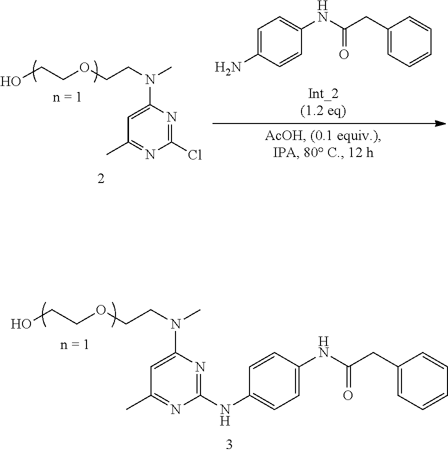

To a mixture of 2-(2-((2-chloro-6-methylpyrimidin-4-yl)(methyl)amino)ethoxy)ethan-1-ol (2) (1.4 g, 5.70 mmol) and N-(4-aminophenyl)-2-phenyl-acetamide (Int_2) (1.55 g, 6.84 mmol) in IPA (2 mL) was added AcOH (34.22 mg, 0.569 mmol, 32.59 uL). The mixture was stirred at 80° C. for 12 h. LCMS showed the reaction was completed and the desired product was detected. The reaction mixture was diluted with water (20 mL) and extracted with DCM (3×20 mL). The organic combined organic layers were washed with brine (3×20 mL) and dried over Na₂SO₄. After filtered, the filtrate was concentrated under reduced pressure to give the crude product. The crude product was purified by column chromatography on silica gel (eluted with PE: EA=100:1 to 0:1) to give N-[4-[[4-[2-(2-hydroxyethoxy)ethyl-methyl-amino]-6-methyl-pyrimidin-2-yl]amino]phenyl]-2-phenylacetamide (3) (1.3 g, yield 52.39%) was obtained as white solid. LCMS (ESI+): m/z=436.3 (M+H)⁺, RT: 0.790 min.

### General Procedure for Preparation of 4A

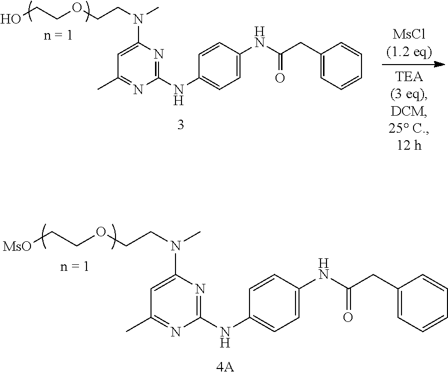

To a solution of N-[4-[[4-[2-(2-hydroxyethoxy)ethyl-methyl-amino]-6-methyl-pyrimidin-2-yl]amino]phenyl]-2-phenyl-acetamide (1.3 g, 2.98 mmol) in DCM (13 mL) was added MsCl (410.32 mg, 3.58 mmol,) and TEA (906.14 mg, 8.95 mmol, 1.25 mL) at 0° C. Then the mixture was stirred at 25° C. for 12 h. TLC showed the reaction was completed and the desired point was detected. The reaction mixture was diluted with water (20 mL) and extracted with DCM (3×20 mL). The organic combined organic layers were washed with brine (3×20 mL) and dried over Na₂SO₄. After filtered, the filtrate was concentrated under reduced pressure to give the crude product. The crude product was purified by column chromatography on silica gel (eluted with PE: EA=100:1 to 0:1) to give 2-(2-(methyl(6-methyl-2-((4-(2-phenylacetamido)phenyl)amino)pyrimidin-4-yl)amino)ethoxy)ethyl methanesulfonate (4A) (1.2 g, yield 78.27%) as white solid.

¹H NMR (400 MHz, DMSO-d₆) δ=9.98 (s, 1H), 8.91 (s, 1H), 7.64 (br d, J=8.9 Hz, 2H), 7.43 (d, J=8.9 Hz, 2H), 7.39-7.30 (m, 4H), 7.30 (br d, J=1.8 Hz, 2H), 4.28 (br d, J=4.5 Hz, 2H), 3.76-3.59 (m, 8H), 3.13 (s, 3H), 3.04 (s, 3H), 2.17 (s, 3H)

### General Procedure for Preparation of 5

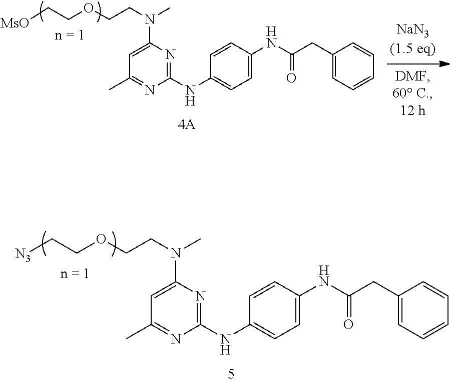

To a solution of 2-[2-[methyl-[6-methyl-2-[4-[(2-phenylacetyl)amino]anilino]pyrimidin-4-yl]amino]ethoxy]ethylmethanesulfonate (4A) (1.2 g, 2.34 mmol) in DMF (12 mL) was added NaN₃ (227.83 mg, 3.50 mmol). Then the mixture was stirred at 60° C. for 12 h. LCMS showed the reaction was completed and the desired product was detected. The reaction mixture was diluted with water (20 mL) and extracted with DCM (3×20 mL). The organic combined organic layers were washed with brine (3×20 mL) and dried over Na₂SO₄. After filtered, the filtrate was concentrated under reduced pressure to give the crude product. The crude product was purified by column chromatography on silica gel (eluted with PE: EA=100:1 to 0:1) to give N-[4-[[4-[2-(2-azidoethoxy)ethyl-methyl-amino]-6-methyl-pyrimidin-2-yl]amino]phenyl]-2-phenylacetamide (5) (0.9 g, yield 83.64%) as white solid.

LCMS (ESI+): m/z=461.2 (M+H)⁺, RT: 0.836 min.

### General Procedure for Preparation Of 6

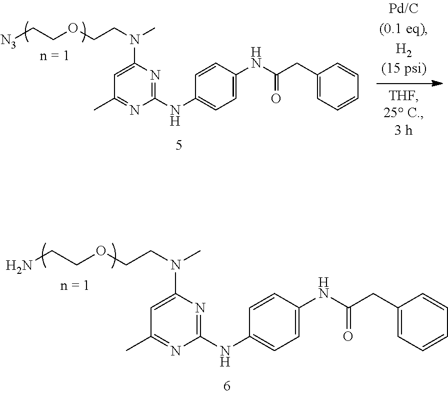

To a solution of N-[4-[[4-[2-(2-azidoethoxy)ethyl-methyl-amino]-6-methyl-pyrimidin-2-yl]amino]phenyl]-2-phenylacetamide (5) (0.85 g, 1.85 mmol) in THF (9 mL) were added Pd/C (196.42 mg, 0.185 mmol, 10% purity) under N₂. The suspension was degassed under vacuum and purged with H₂ several times. The mixture was stirred under H₂ (15 psi) at 25° C. for 3 h. TLC showed the reaction was completed and the desired point was detected. The reaction mixture was filtered and the filter was concentrated to give N-(4-((4-((2-(2-aminoethoxy)ethyl)(methyl)amino)-6-methylpyrimidin-2-yl)amino)phenyl)-2-phenylacetami de (6) (0.34 g, yield 42.39%) as white solid.

¹H NMR (400 MHz, DMSO-d₆) δ=9.97 (s, 1H), 8.90 (s, 1H), 7.64 (d, J=9.0 Hz, 2H), 7.42 (d, J=9.0 Hz, 2H), 7.36-7.18 (m, 6H), 3.63-3.58 (m, 4H), 3.58-3.54 (m, 2H), 3.42-3.33 (m, 4H), 3.03 (s, 3H), 2.17 (s, 3H), 1.23 (s, 2H)

### General Procedure for Preparation of NUCC-0227323

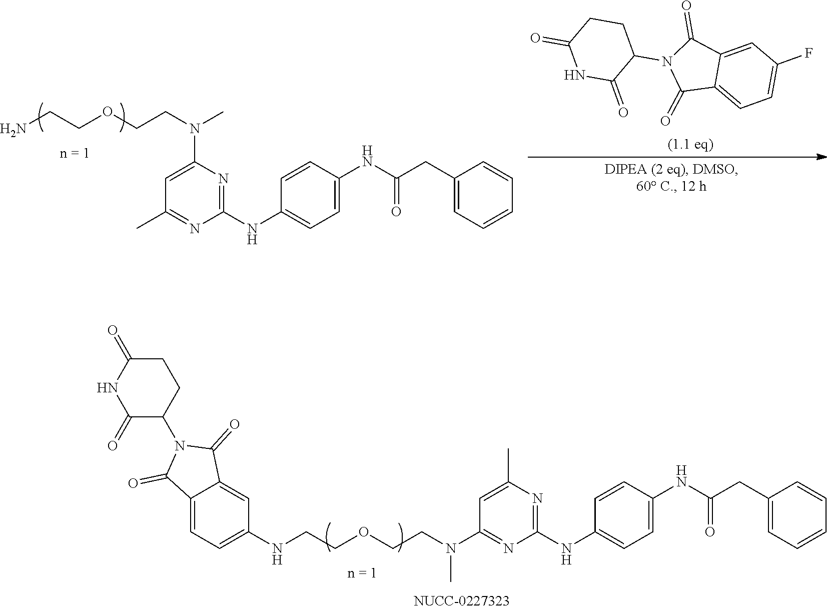

To a solution of N-(4-((4-((2-(2-aminoethoxy)ethyl)(methyl)amino)-6-methylpyrimidin-2-yl)amino)phenyl)-2-phenylacetamide (6) (0.34 g, 0.782 mmol) and 2-(2,6-dioxo-3-piperidyl)-5-fluoro-isoindoline-1,3-dione (237.74 mg, 0.861 mmol) in DMSO (3 mL) was added DIEA (202.25 mg, 1.56 mmol) at 20° C. Then the mixture was stirred at 60° C. for 12 h. LCMS showed the reaction was completed and the desired product was detected. The reaction mixture was concentrated under reduced pressure to give a crude product. The residue was purified by prep-HPLC (NH₄HCO₃ Condition) to afford N-(4-((4-((2-(2-((2-(2,6-dioxopiperidin-3-yl)-1,3-dioxoisoindolin-5-yl)amino)ethoxy)ethyl)(methyl)amino)-6methylpyrimidin-2-yl)amino)phenyl)-2-phenylacetamide (NUCC-0227323) (60.0 mg, yield 11.10%) as white solid.

LCMS (ESI+): m/z=691.3 (M+H)⁺, RT: 2.830 min.

¹H NMR (400 MHz, DMSO-d₆) δ=12.68-12.16 (m, 1H), 11.08 (s, 1H), 10.30 (br s, 1H), 9.99-9.63 (m, 1H), 7.63 (br d, J=8.6 Hz, 2H), 7.53 (d, J=8.3 Hz, 1H), 7.43 (br s, 2H), 7.38-7.29 (m, 4H), 7.28-7.20 (m, 1H), 7.12 (br s, 1H), 6.98 (s, 1H), 6.85 (br s, 1H), 6.57-6.27 (m, 1H), 5.14-4.94 (m, 1H), 3.85-3.69 (m, 3H), 3.65 (s, 5H), 3.57-3.49 (m, 2H), 3.23-3.05 (m, 3H), 2.96-2.79 (m, 1H), 2.60 (br s, 1H), 2.37-2.21 (m, 3H), 2.07-1.91 (m, 1H)

NUCC-0227324 and NUCC-0227325 were synthesized following similar steps as those applied to NUCC-0227323.

### Spectra of NUCC-0227324:

LCMS (ESI+): m/z=779.3 (M+H)⁺, RT: 2.226 min.

1H NMR (400 MHz, METHANOL-d4) 6=7.65-7.57 (m, 2H), 7.49-7.32 (m, 7H), 7.30-7.22 (m, 1H), 6.94 (dd, J=1.9, 8.5 Hz, 1H), 6.83-6.75 (m, 1H), 6.41-6.21 (m, 1H), 5.03 (td, J=5.2, 12.6 Hz, 1H), 3.85-3.79 (m, 1H), 3.75-3.69 (m, 4H), 3.67-3.61 (m, 6H), 3.59-3.52 (m, 5H), 3.31-3.15 (m, 5H), 2.88-2.62 (m, 3H), 2.35-2.29 (m, 3H), 2.14-1.99 (m, 1H)

### Spectra of NUCC-0227325:

LCMS (ESI+): m/z=867.4 (M+H)⁺, RT: 2.232 min.

1H NMR (400 MHz, METHANOL-d4) 6=7.63 (d, J=8.9 Hz, 2H), 7.54 (d, J=8.3 Hz, 1H), 7.49-7.41 (m, 2H), 7.40-7.31 (m, 4H), 7.30-7.24 (m, 1H), 7.00 (s, 1H), 6.87 (dd, J=2.0, 8.4 Hz, 1H), 6.42-6.27 (m, 1H), 5.05 (dd, J=5.5, 12.5 Hz, 1H), 3.81 (t, J=5.3 Hz, 1H), 3.73-3.70 (m, 3H), 3.69-3.63 (m, 8H), 3.62-3.57 (m, 5H), 3.57-3.46 (m, 7H), 3.37-3.34 (m, 2H), 3.24-3.18 (m, 3H), 2.90-2.78 (m, 1H), 2.77-2.64 (m, 2H), 2.39-2.30 (m, 3H), 2.14-2.01 (m, 1H).

We claim:

1. A compound, or a pharmaceutically acceptable salt thereof, the compound having a formula: M_{TG2}-L-M_{E3}, wherein M_{TG2} is a moiety that binds to tissue transglutaminase 2 (TG2), L is a bond or a linker covalently attaching M_{TG2} and M_{E3}, and M_{E3} is a moiety that binds to an E3 ubiquitin ligase.

2. The compound of claim 1, wherein M_{TG2} has a formula:

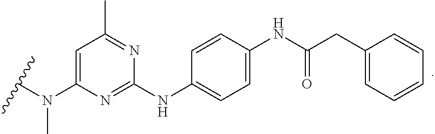 [SMILES: *N(C)c1cc(C)nc(Nc2ccc(NC(=O)Cc3ccccc3)cc2)n1]

3. The compound of claim 1, wherein L comprises a polyethylene glycol moiety.

4. The compound of claim 1, wherein L is selected from the group consisting of 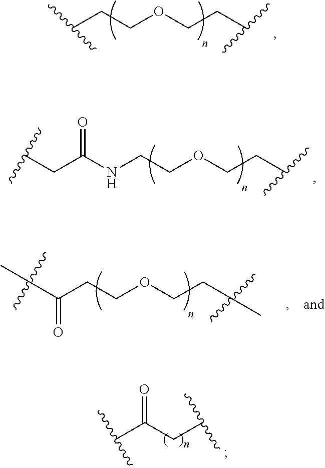
and
wherein n is an integer from 1 to 20.

5. The compound of claim 4, wherein n is an integer from 1 to 5.

6. The compound of claim 1, wherein L is selected from the group consisting of

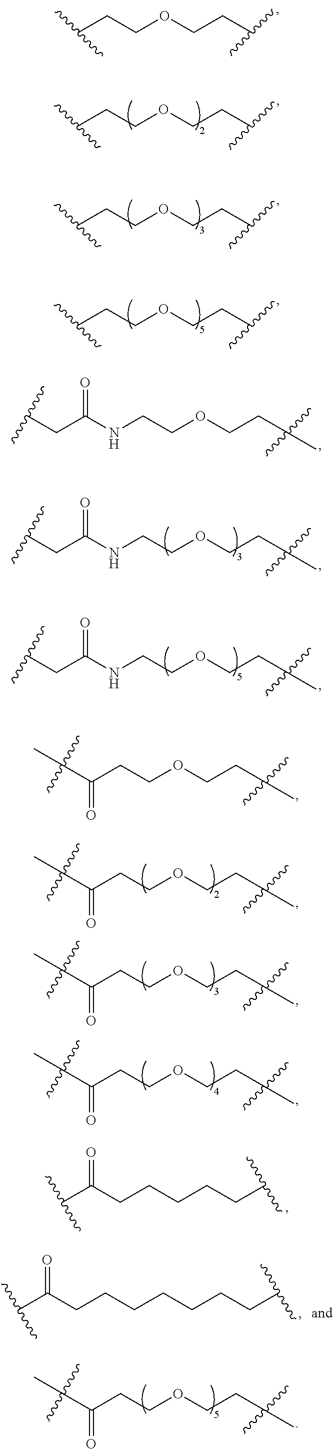

7. The compound of claim 1, wherein M_{E3} is a moiety that binds to an E3 ubiquitin ligase selected from Von Hippel Lindau (VHL) E3 ubiquitin ligase, cereblon (CRBN) E3 ubiquitin ligase, inhibitor of apoptosis protein (IAP) E3 ubiquitin ligase, and mouse double minute 2 homolog (MDM2) E3 ubiquitin ligase.

8. The compound of claim 1, wherein M_{E3} has a formula selected from the group consisting of:

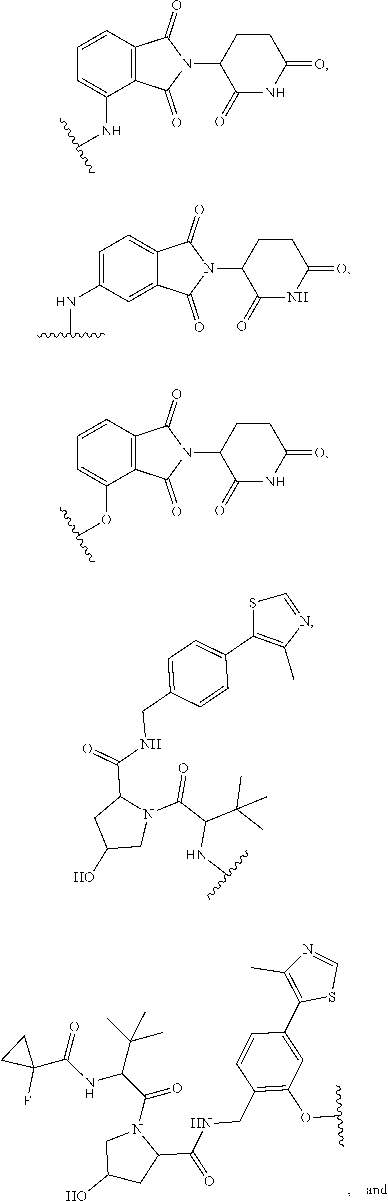 [SMILES: *Nc1cccc2c1C(=O)N(C1CCC(=O)NC1=O)C2=O ; *Nc1ccc2c(c1)C(=O)N(C1CCC(=O)NC1=O)C2=O ; *Oc1cccc2c1C(=O)N(C1CCC(=O)NC1=O)C2=O ; *NC(C(=O)N1CC(O)CC1C(=O)NCc1ccc(-c2scnc2C)cc1)C(C)(C)C ; *Oc1cc(-c2scnc2C)ccc1CNC(=O)C1CC(O)CN1C(=O)C(NC(=O)C1(F)CC1)C(C)(C)C]

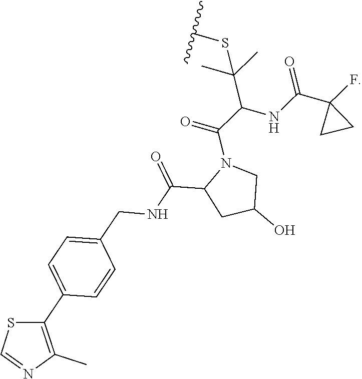 [SMILES: *SC(C)(C)C(NC(=O)C1(F)CC1)C(=O)N1CC(O)CC1C(=O)NCc1ccc(-c2scnc2C)cc1]

9. The compound of claim 1, wherein the compound has a formula I

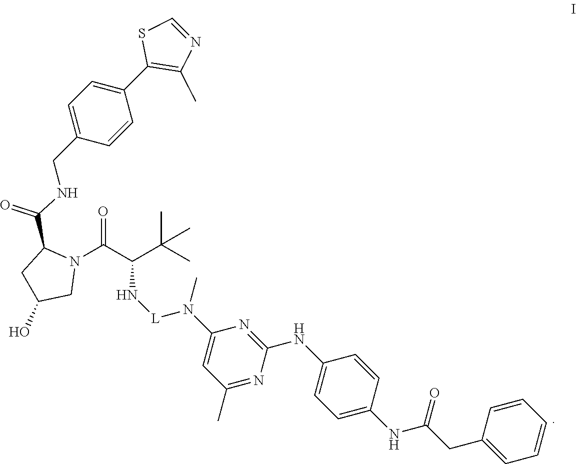

10. The compound of claim 9, wherein L is 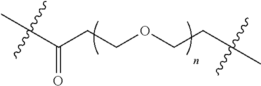
and n is an integer from 1 to 5.

11. The compound of claim 1, wherein the compound has a formula selected from the group consisting of

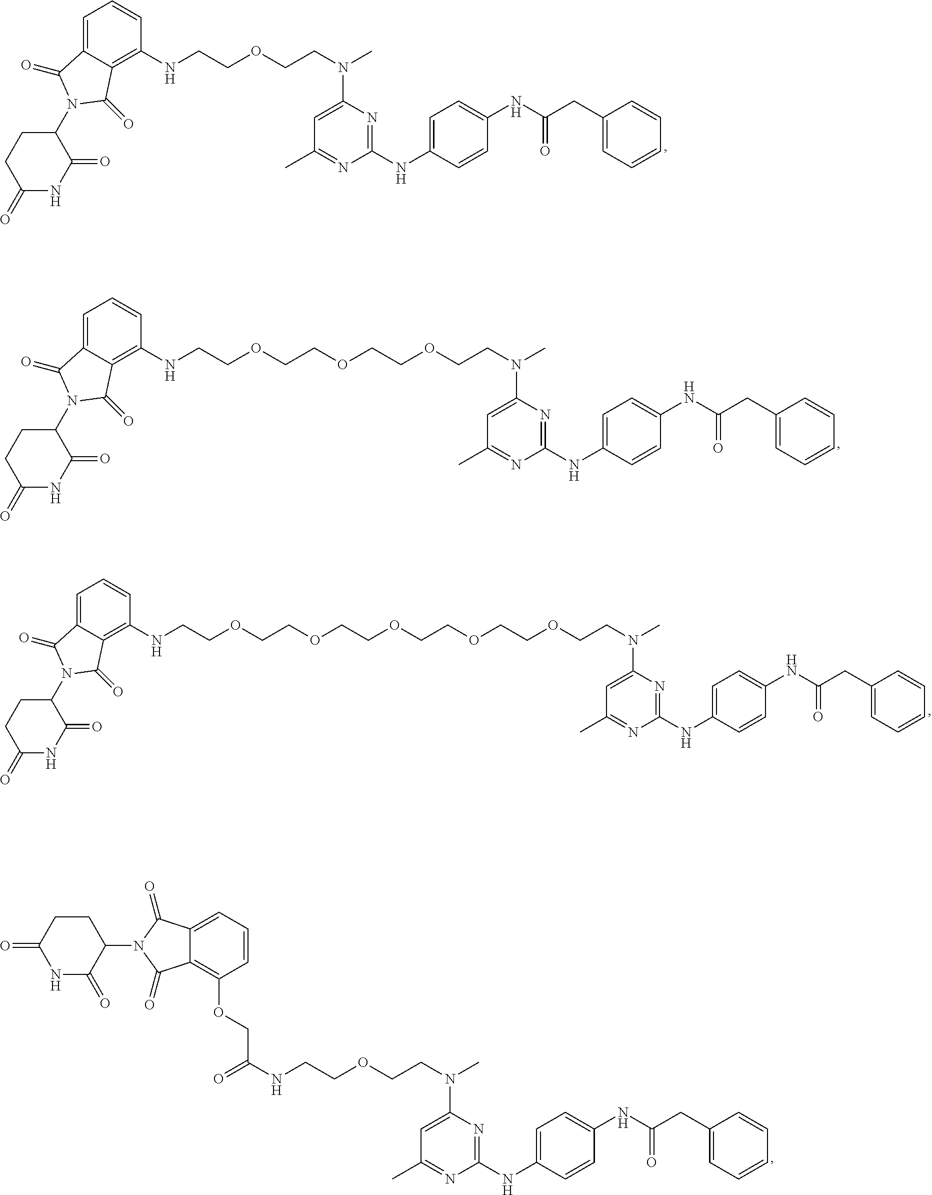 [SMILES: Cc1cc(N(C)CCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNc2cccc3c2C(=O)N(C2CCC(=O)NC2=O)C3=O)nc(Nc2ccc(NC(=O)Cc3ccccc3)cc2)n1 ; Cc1cc(N(C)CCOCCNC(=O)COc2cccc3c2C(=O)N(C2CCC(=O)NC2=O)C3=O)nc(Nc2ccc(NC(=O)Cc3ccccc3)cc2)n1]

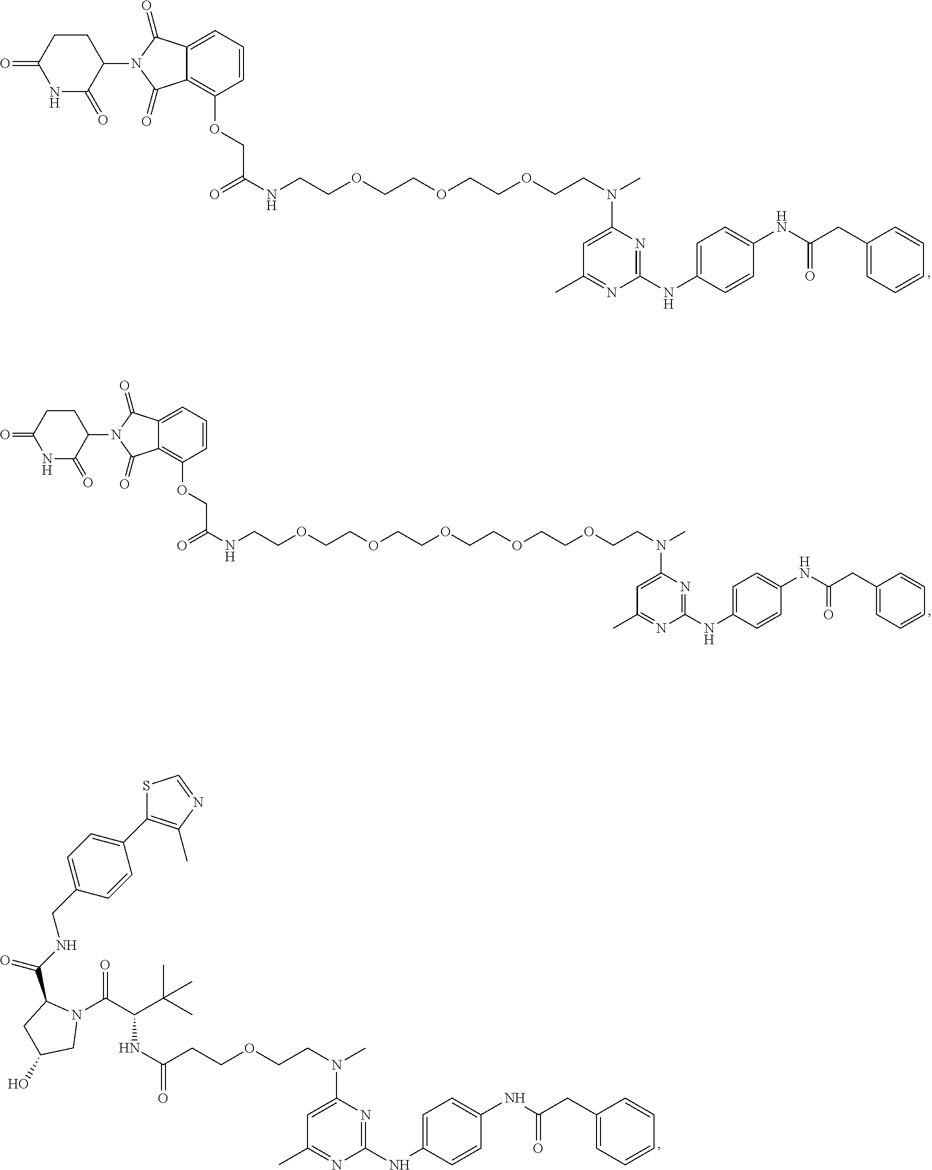 [SMILES: Cc1cc(N(C)CCOCCOCCOCCNC(=O)COc2cccc3c2C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNC(=O)COc2cccc3c2C(=O)N(C2CCC(=O)NC2=O)C3=O)nc(Nc2ccc(NC(=O)Cc3ccccc3)cc2)n1 ; Cc1cc(N(C)CCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

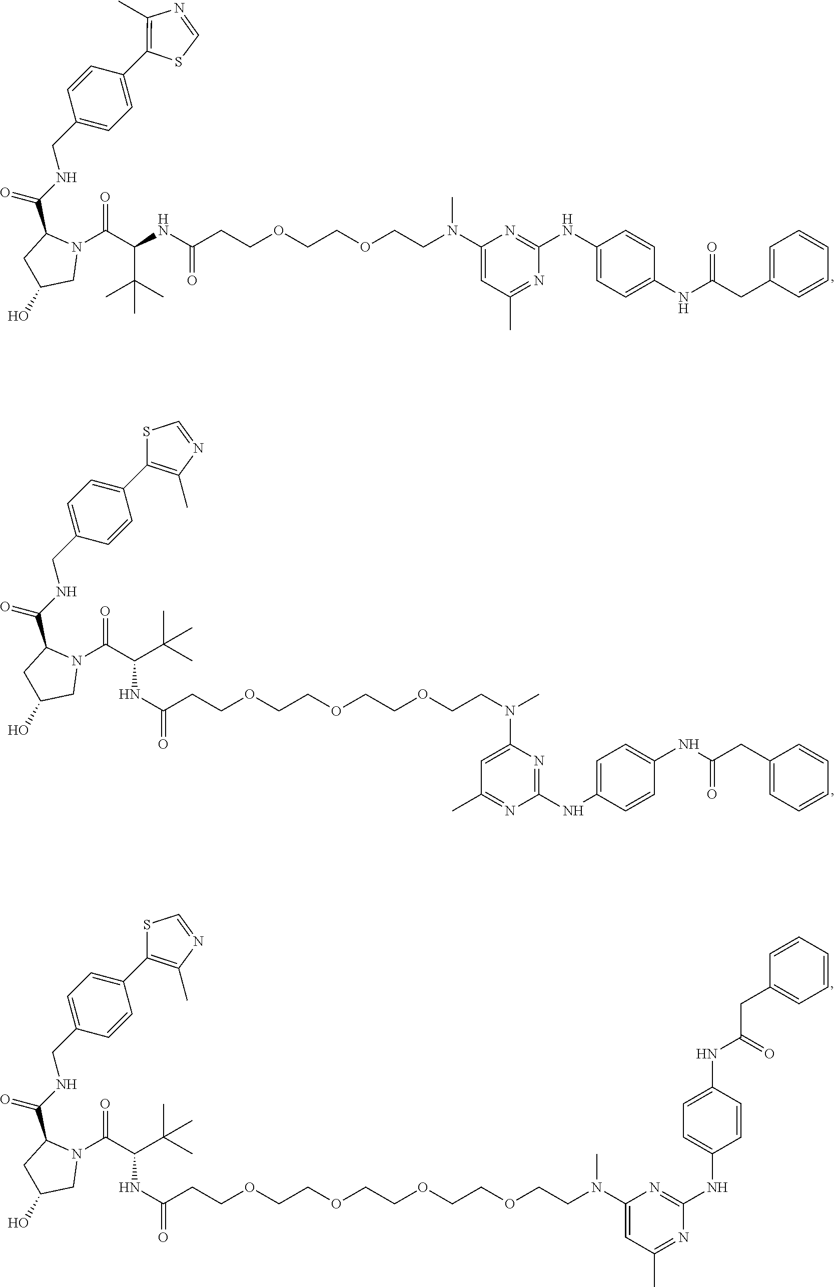 [SMILES: Cc1cc(N(C)CCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCOCCOCCOCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

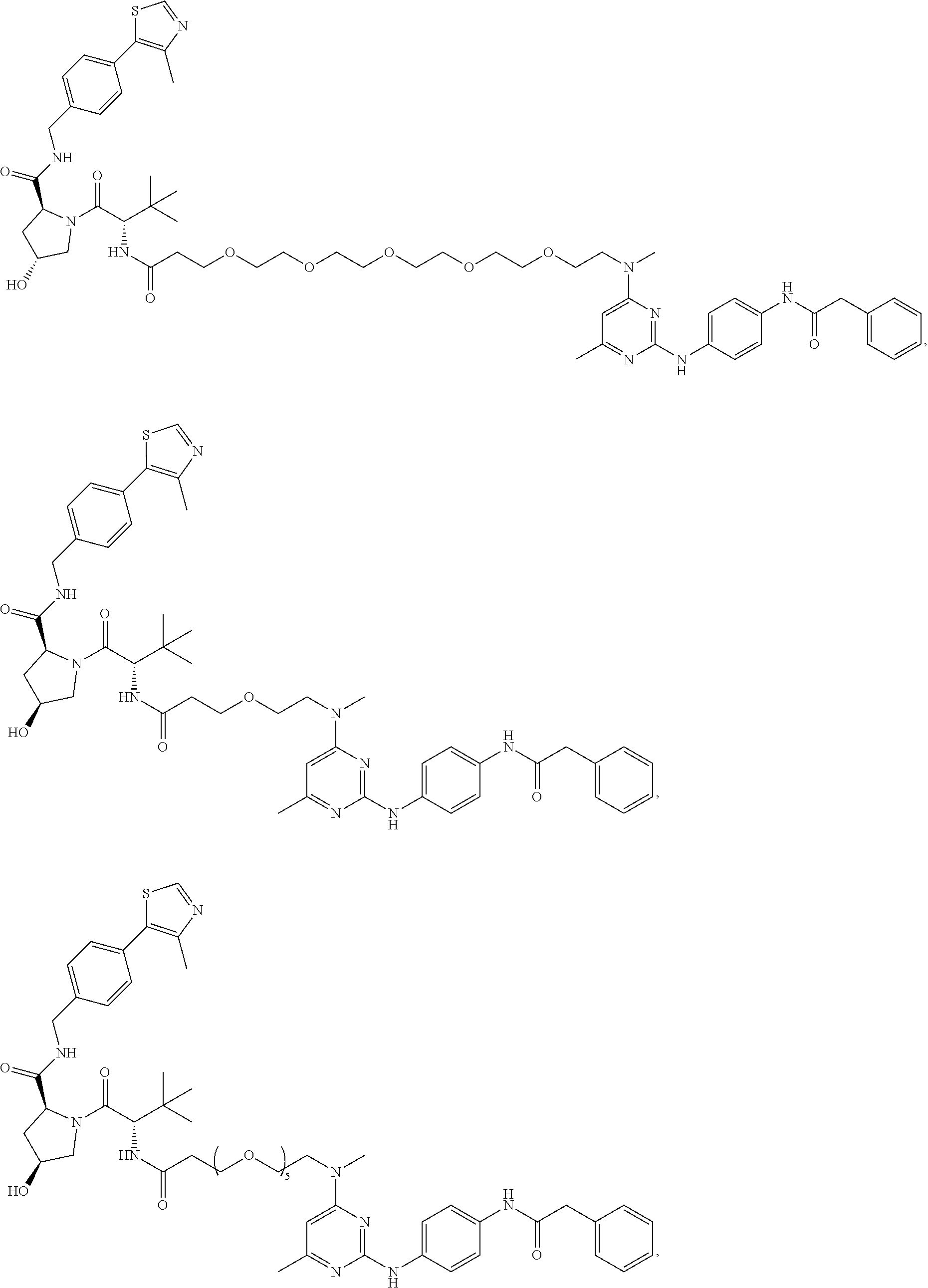

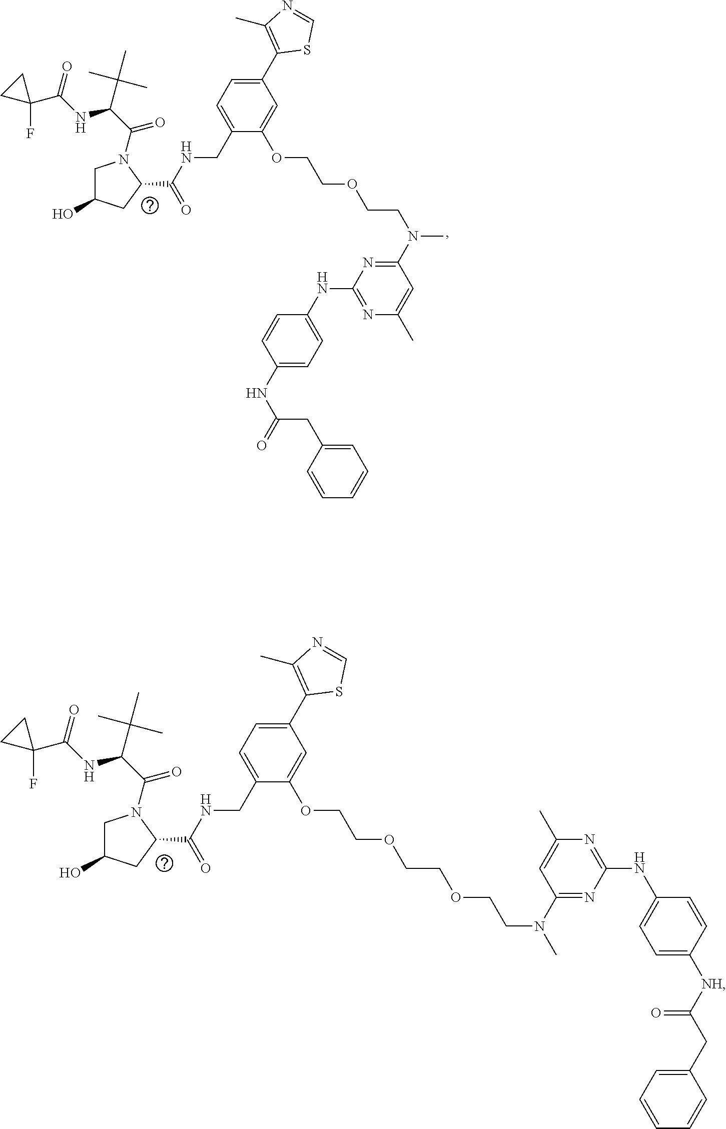 [SMILES: Cc1cc(N(C)CCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1 ; Cc1cc(N(C)CCOCCOCCOc2cc(-c3scnc3C)ccc2CNC(=O)[C@@H]2C[C@@H](O)CN2C(=O)[C@@H](NC(=O)C2(F)CC2)C(C)(C)C)nc(Nc2ccc(NC(=O)Cc3ccccc3)cc2)n1]

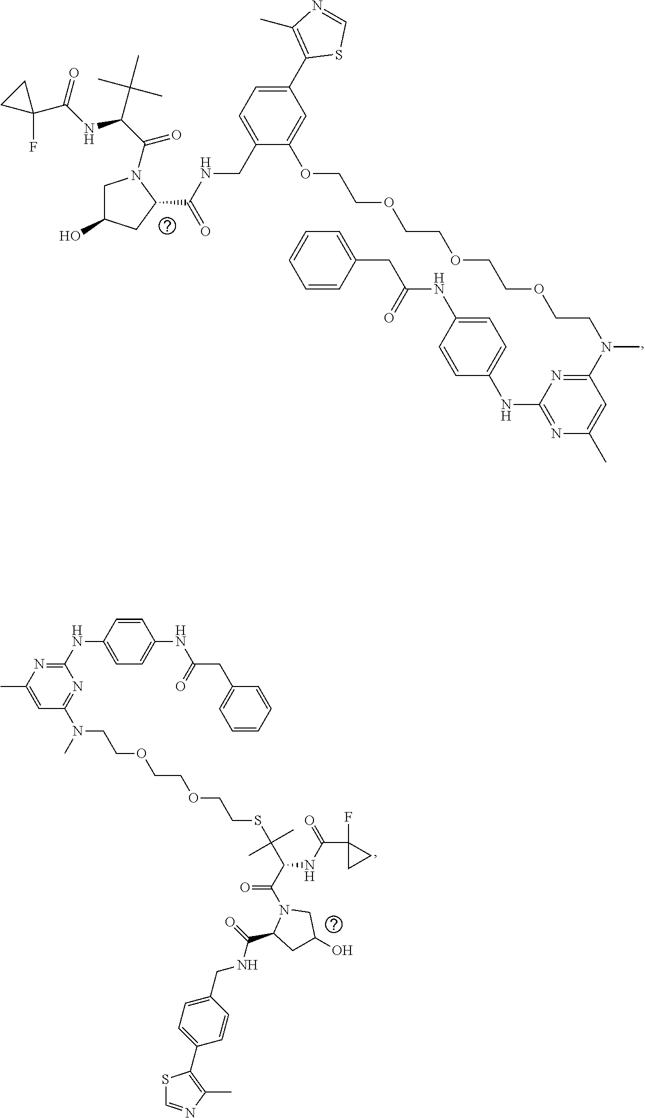

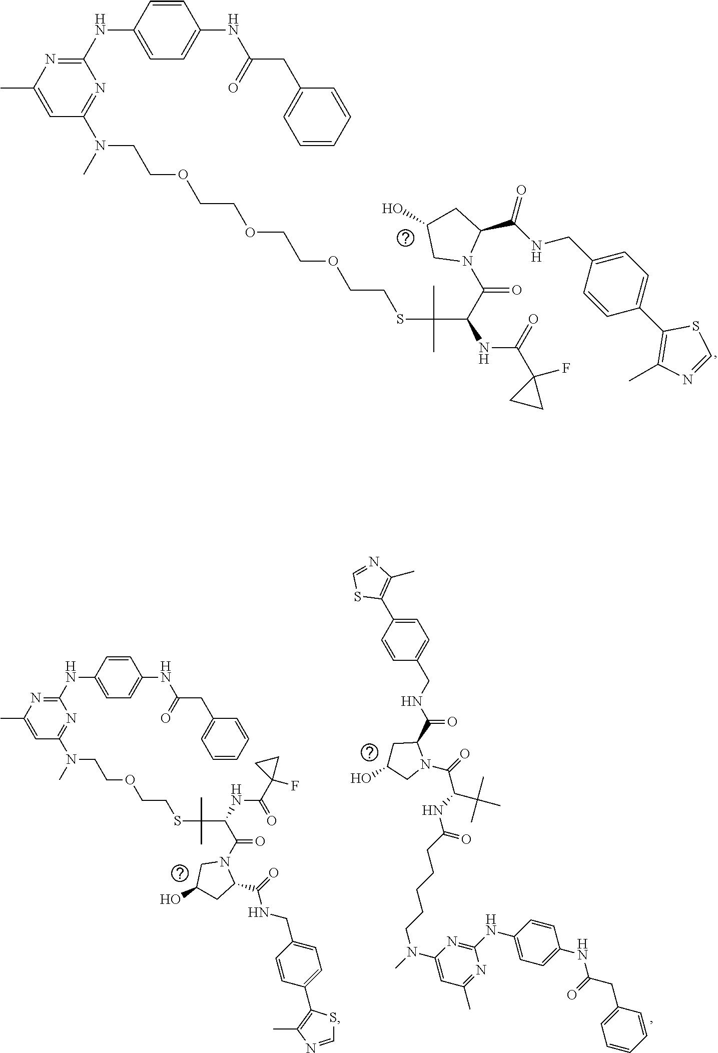 [SMILES: Cc1cc(N(C)CCOCCOCCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1 ; Cc1cc(N(C)CCOCCSC(C)(C)[C@H](NC(=O)C2(F)CC2)C(=O)N2C[C@H](O)C[C@H]2C(=O)NCc2ccc(-c3scnc3C)cc2)nc(Nc2ccc(NC(=O)Cc3ccccc3)cc2)n1 ; Cc1cc(N(C)CCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1]

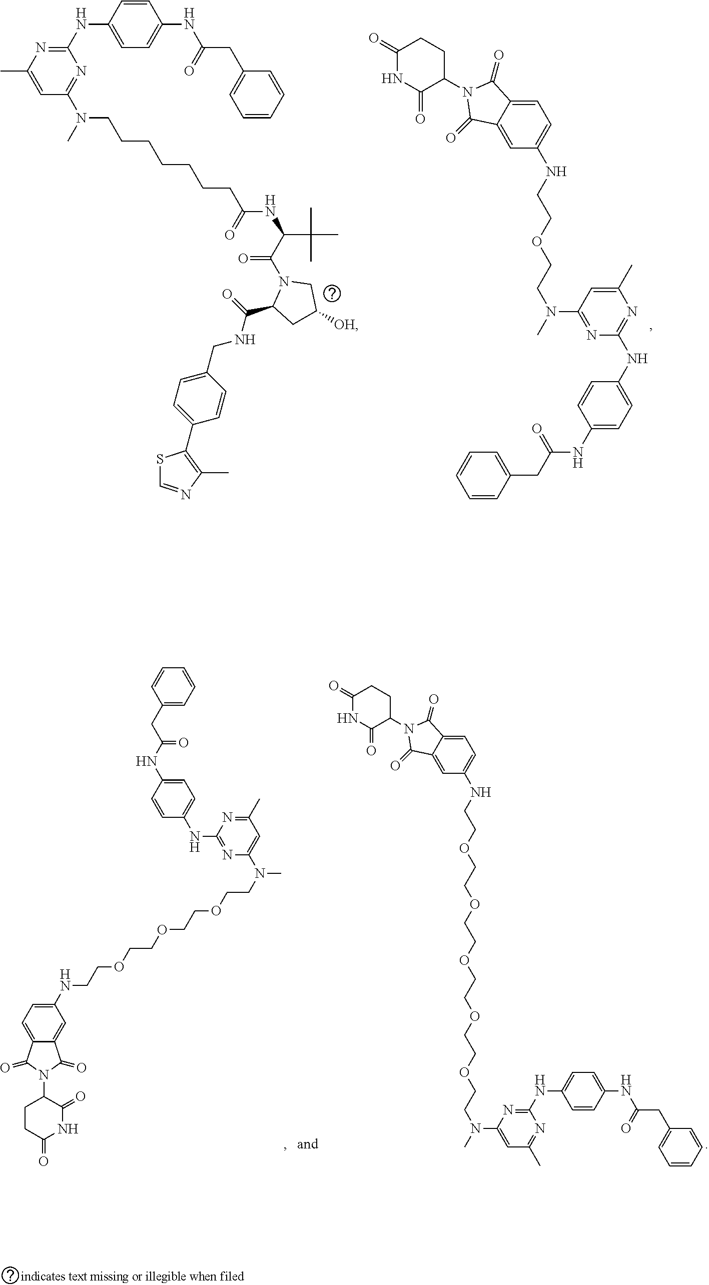 [SMILES: Cc1cc(N(C)CCCCCCCC(=O)N[C@H](C(=O)N2C[C@H](O)C[C@H]2C(=O)NCc2ccc(-c3scnc3C)cc2)C(C)(C)C)nc(Nc2ccc(NC(=O)Cc3ccccc3)cc2)n1 ; Cc1cc(N(C)CCOCCNc2ccc3c(c2)C(=O)N(C2CCC(=O)NC2=O)C3=O)nc(Nc2ccc(NC(=O)Cc3ccccc3)cc2)n1 ; Cc1cc(N(C)CCOCCOCCOCCNc2ccc3c(c2)C(=O)N(C2CCC(=O)NC2=O)C3=O)nc(Nc2ccc(NC(=O)Cc3ccccc3)cc2)n1 ; Cc1cc(N(C)CCOCCOCCOCCOCCOCCNc2ccc3c(c2)C(=O)N(C2CCC(=O)NC2=O)C3=O)nc(Nc2ccc(NC(=O)Cc3ccccc3)cc2)n1]

12. The compound of claim 1, wherein the compound has a formula

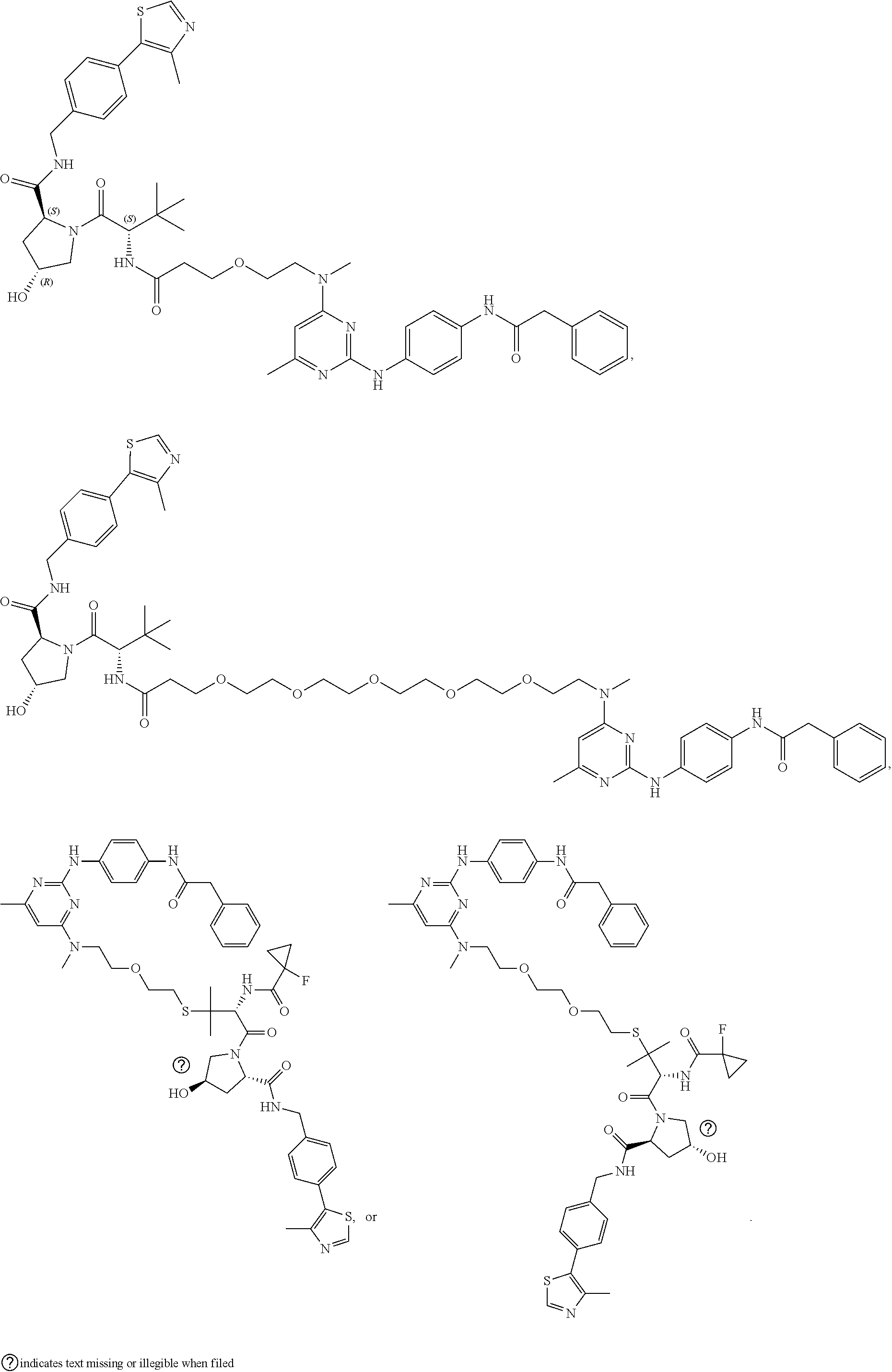

13. A pharmaceutical composition comprising a therapeutically effective amount of the compound of claim 1, or a pharmaceutically acceptable salt thereof, and a pharmaceutically acceptable carrier, excipient, or diluent.

14. A method of treating a disease or disorder associated with TG2 activity in a subject in need thereof, the method comprising administering to the subject the pharmaceutical composition of claim 13.

15. The method of claim 14, wherein the disease or disorder is a cell proliferative disease or disorder.

16. The method of claim 15, wherein the cell proliferative disease or disorder is ovarian cancer, melanoma, cervical cancer, colorectal cancer, liver cancer, meningioma, neuroblastomoa, prostate cancer, pancreatic cancer, lung cancer, breast cancer, or glioblastoma.

17. The method of claim 16, wherein the cell proliferative disease or disorder is ovarian cancer.

18. A method of inhibiting cell proliferation, the method comprising contacting cells with the pharmaceutical composition of claim 13.

19. The method of claim 18, wherein the cells are cells of ovarian cancer, melanoma, cervical cancer, colorectal cancer, liver cancer, meningioma, neuroblastomoa, prostate cancer, pancreatic cancer, lung cancer, breast cancer, or glioblastoma.

20. The method of claim 19, wherein the cells are ovarian cancer cells.

21. The method of claim 18, wherein the contact is in vitro.
